(19) United States
(12) Patent Application Publication
(10) Pub. No.: US 2007/0003552 A1
(43) Pub. Date: Jan. 4, 2007

(54) Cross-beta structure comprising amyloid binding proteins and methods for detection of the cross-beta structure, for modulating cross-beta structures fibril formation and for modulating cross-beta structure-mediated toxicity and method for interfering with blood coagulation

(76) Inventors: Martijn F. B. G. Gebbink, Eemnes (NL); Barend Bouma, Houten (NL); Onno W. Kranenburg, Amsterdam (NL); Louise M. J. Kroon, Bunnik (NL)

Correspondence Address: TRASK BRITT, P.O. BOX 2550, SALT LAKE CITY, UT, 84110, US

(21) Appl. No.: 11/087,102

(22) Filed: Mar. 21, 2005

Related U.S. Application Data
(63) Continuation-in-part of application No. 11/033,105, filed on Jan. 10, 2005.
Application No. 11/033,105 is a continuation of application No. PCT/NL03/00501, filed on Jul. 8, 2003.

(30) Foreign Application Priority Data
Jul. 9, 2002 (EP) EP02077797.5

(51) Int. Cl. A61K 39/395

(52) U.S. Cl. 424/146.1

(57) ABSTRACT

The invention relates to the field of biochemistry, molecular biology, structural biology and medicine. More in particular, the invention relates to cross-β structures and the biological role of these cross-β structures.

### CROSS-REFERENCE TO RELATED APPLICATIONS

This application is a continuation-in-part of U.S. patent application Ser. No. 11/033,105, filed Jan. 10, 2005, pending, which application is a continuation of PCT International Patent Application PCT/NL2003/000501, filed Jul. 8, 2003, designating the United States of America, corresponding to PCT International Publication WO 2004/004698 A3 (published in English on Jan. 15, 2004), which claims priority from European Patent Application EP02077797.5, filed Jul. 9, 2002, the contents of the entirety of all of which are incorporated by this reference.

### TECHNICAL FIELD

The invention relates to the field of biochemistry, molecular biology, structural biology and medicine. More in particular, the invention relates to cross-β structure, their binding proteins and their biological roles.

### BACKGROUND

An increasing body of evidence suggests that unfolding of globular proteins can lead to toxicity.¹ Unfolded proteins can initiate protein aggregation and fibrillization by adopting a partially structured conformation. Such fibrillar aggregates can (slowly) accumulate in various tissue types and are associated with a variety of degenerative diseases. The term “amyloid” is used to describe these fibrillar deposits (or plaques). Diseases characterized by anyloid are referred to as amyloidosis and include Alzheimer disease (AD), light-chain amyloidosis, type II diabetes and spongiform encephalopathies. It has been found recently that toxicity is an inherent property of misfolded proteins. According to the present invention this common mechanism for these conformational diseases.¹

A cross-β structure is a secondary structural element in peptides or proteins. A cross-β structure can be formed upon denaturation, proteolysis or unfolding of proteins.² These secondary structure elements are typically absent in globular regions of proteins. The cross-β structure is found in amyloid fibrils. Amyloid peptides or proteins are cytotoxic to cells. A cross-β structure is composed of stacked β-sheets. In a cross-β structure the individual β-strands, run either perpendicular to the long axis of a fibril, or the β-strands run in parallel to the long axis of a fiber. The direction of the stacking of the β-sheets in cross-β structures is perpendicular to the long fiber axis.

### DISCLOSURE OF THE INVENTION

We report here that glycation of proteins also induces the formation of the cross-β structure. Our results, combined with existing literature information indicate that a common structure is induced upon unfolding of globular proteins. Therefore, the present invention discloses a novel pathway involving cross-β structure, which pathway will be called “cross-β structure pathway.” This pathway consists of several cross-β structure binding proteins, including so-called multiligand receptors and is involved in protein degradation and/or protein clearance. We also report the identification of novel cross-β binding proteins that contain a cross-β structure binding module. These findings support the identification of a cross-β structure pathway. Multiple aspects of this novel pathway are outlined below.

For example, the present invention discloses that proteolysed, denatured, unfolded, glycated, oxidized, acetylated or otherwise structurally altered proteins adopt cross-β structures. Examples of known cross-β structure forming proteins are all proteins that cause amyloidosis or proteins that are found in disease related amyloid depositions, for example, but not restricted to, Alzheimer β-amyloid (Aβ) and Islet Amyloid PolyPeptide (IAPP). The present invention discloses that fibrin, glycated proteins (for example glycated albumin and glycated hemoglobin) and endostatin are also capable of adopting a cross-β structure.

The invention furthermore discloses the identification of the formation of a cross-β structure as a signal for protein degradation and/or protein clearance.

The serine protease tissue plasminogen activator (tPA) induces the formation of plasmin through cleavage of plasminogen. Plasmin cleaves fibrin and this occurs during lysis of a blood clot. Although not essential for fibrinolysis in mice,^{3; 4} tPA has been recognized for its role in fibrinolysis for a long time. ^{5; 6} Activation of plasminogen by tPA is stimulated by fibrin or fibrin fragments, but not by its precursor, fibrinogen.⁷⁻¹⁰ This can be in part explained by the strong binding of tPA to fibrin and weak binding to fibrinogen. The binding sites in fibrin and in tPA responsible for binding and activation of tPA have been mapped and studied in detail.⁸⁻²¹ However the exact structural basis for the interaction of tPA with fibrin was unknown. In addition to fibrin and fibrin fragments, many other proteins have been described that are similarly capable of binding tPA and stimulating tPA-mediated plasmin formation.²²⁻³⁶ Like with fibrin and fibrin fragments, the exact nature of the interaction(s) between these ligands for tPA and tPA were not known. Moreover, it was unknown why and how all these proteins, which lack primary sequence homology, bind tPA. The invention now discloses tissue type plasminogen activator (tPA) as a protein capable of binding cross-β structures. Furthermore, the invention discloses the finger domain (also named fibronectin type I domain) and other comparable finger-domains as a cross-β structure binding module. The present invention further discloses that proteins which bind to these fingers will be typically capable of forming cross-β structures.

Since fibrin contains the cross-β structure, the present invention also discloses that the generation of cross-β structures plays a role in physiological processes. The invention discloses that the generation of cross-β structures is part of a signaling pathway, the “cross-β structure pathway,” that regulates protein degradation and/or protein clearance. Inadequate function of this pathway may result in the development of diseases, such as conformational diseases³⁷ and/or amyloidosis.

The present invention furthermore discloses that the cross-β structure is a common denominator in ligands for multiligand receptors.³⁸ The invention discloses therefore that multiligand receptors belong to the “cross-β structure pathway.”

The best studied example of a receptor for a cross-β structure is RAGE.³⁹⁻⁴⁴ Examples of ligands for RAGE are Aβ, protein-advanced glycation end-products (AGE) adducts (including glycated-BSA), amphoterin and S100. RAGE is a member of a larger family of multiligand receptors,³⁸ that includes several other receptors, some of which, including CD36 are known to bind cross-β structure containing proteins (see also FIG. 1). At present it is not clear what the exact nature of the structure or structures is in the ligands of these receptors that mediates the binding to these receptors. We report here that glycation of proteins also induces the formation of a cross-β structure. Therefore, we disclose that all these receptors form part of a mechanism to deal with the destruction and removal of unwanted or even damaging proteins or agents. These receptors play a role in recognition of infectious agents or cells, recognition of apoptotic cells and in internalization of protein complexes and/or pathogens. It is furthermore disclosed that all these receptors recognize the same or similar structure, the cross-β structure, to respond to undesired molecules. We show that tPA binds cross-β structures, providing evidence that tPA belongs to the multiligand receptor family. As disclosed herein, tPA and the other multiligand receptors bind the β structure and participate in the destruction of unwanted biomolecules. A prominent role of the protease tPA in the pathway lies in its ability to initiate a proteolytic cascade that includes the formation of plasmin. Proteolysis is likely to be essential for the degradation and subsequent removal of extracellular matrix components. The effect of tPA on the extracellular matrix will affect cell adhesion, cell migration, cell survival and cell death, through for example integrin mediated processes. Based on our studies we have provided strong evidence that at least three other proteins, FXII a.k.a. FXII (factor XII), hepatocyte growth factor activator (HGFa) and fibronectin, that contain one or more finger domain(s) are also part of the “cross-β structure pathway.”

Especially the role of FXII is important, since it activates the intrinsic coagulation pathway. Activation of the intrinsic pathway, and the resulting formation of vasoactive peptides and the activation of other important proteins contribute to the process of protection and/or clearance of undesired proteins or agents. The “cross-β structure pathway” is modulated in many ways. Factors that regulate the pathway include modulators of synthesis and secretion, as well as modulators of activity. The pathway is involved in many physiological and pathological processes. Therefore, the invention furthermore provides a method for modulating extracellular protein degradation and/or protein clearance comprising modulating the activity of a receptor for cross-β structure forming proteins. Examples of receptors for cross-β structure forming proteins include RAGE, CD36, Low density lipoprotein Related Protein (LRP), Scavenger Receptor B-1 (SR-BI), SR-A. The invention discloses that FXII, HGFa and fibronectine are also receptors for cross-β structure.

The present invention discloses that tissue-type plasminogen activator (tPA) is a cross-β structure binding protein, a multiligand receptor and a member of the “cross-β structure pathway.” The invention discloses that tPA mediates cross-β structure induced cell dysfunction and/or cell toxicity. The invention discloses that tPA mediates at least in part cell dysfunction and/or toxicity through activation of plasminogen. The plasminogen dependent effects are inhibited by B-type carboxypeptidase activity B and thereby a role for carboxyterminal lysine residues in the cross-β structure pathway is disclosed.

The present invention relates, amongst others, to the structure(s) in fibrin and other proteins that bind tPA, to the binding domain in tPA and to the pathway(s) regulated by this structure. The present invention discloses a presence of cross-β structures in proteins and peptides that are capable of binding tPA. The herein disclosed results indicate a strong correlation between the presence of a cross-β structure and the ability of a molecule to bind tPA. Furthermore, the results indicate the presence of an amyloid structure in fibrin. This indicates that under physiological conditions a cross-β structure can form, a phenomenon that has been previously unrecognized. The formation of cross-β structures has thus far only been associated with severe pathological disorders. tPA binds denatured proteins, which indicates that a large number of proteins, if not all proteins, can adopt a conformation containing cross-β structure or cross-β-like structure(s). Taken together, the formation of cross-β structures is likely to initiate and/or participate in a physiological cascade of events, necessary to adequately deal with removal of unwanted molecules, i.e., misfolded proteins, apoptotic cells or even pathogens. FIG. 1 shows a schematic representation of the “cross-β structure pathway.” This pathway regulates the removal of unwanted biomolecules during several processes, including fibrinolysis, formation of neuronal synaptic networks, clearance of used, unwanted and/or destroyed (denatured) proteins, induction of apoptosis and clearance of apoptotic cells and pathogens. If insufficiently or incorrectly regulated or disbalanced, the pathway may lead to severe disease.

Thus in a first embodiment the invention provides a method for modulating extracellular protein degradation and/or protein clearance comprising modulating cross-β structure formation (and/or cross-β structure-mediated activity) of the protein present in the circulation.

There are two major regular protein-folding patterns, which are known as the β-sheet and the a-helix. An antiparallel β-sheet is formed when an extended polypeptide chain folds back and forth upon itself, with each section of the chains running in the direction opposite to that of its immediate neighbours. This gives a structure held together by hydrogen bonds that connect the peptide bonds in neighbouring chains. Regions of a polypeptide chain that run in the same direction form a parallel β-sheet. A cross-β structure is composed of stacked β-sheets. In a cross-β structure the individual β-strands, run either perpendicular to the long axis of a fibril, or the β-strands run in parallel to the long axis of a fiber. The direction of the stacking of the β-sheets in cross-β structures is perpendicular to the long fiber axis. As disclosed herein within the experimental part, a broad range of proteins is capable of adopting a cross-β structure and moreover these cross-β structure comprising proteins are all capable of binding and stimulating tPA and thereby promoting destruction of unwanted or damaging proteins or agents.

An extracellular protein includes a protein present outside a cell or cells.

Protein degradation and/or protein clearance includes the breakdown and removal of unwanted proteins, for example unwanted and/or destroyed (for example denatured) protein. Also included is the removal of unwanted biomolecules during several processes, including fibrinolysis, formation of neuronal synaptic networks, clearance of used, unwanted and/or destroyed (denatured) proteins, induction of apoptosis and clearance of apoptotic cells and pathogens.

The term “in the circulation” is herein defined as a circulation outside a cell or cells, for example, but not restricted to, the continuous movement of blood.

In yet another embodiment the invention provides a method for increasing extracellular protein degradation and/or protein clearance comprising increasing cross-β structure formation and/or cross-β structure-mediated activity of the protein present in the circulation. Increase of cross-β structure formation of a particular protein leads, for example to activation of tPA which in turn induces the formation of plasmin through cleavage of plasminogen and thus results in an increase in the degradation and/or protein clearance.

In a preferred embodiment the invention provides a method for increasing extracellular protein degradation and/or protein clearance comprising providing a compound capable of increasing cross-β structure formation (and/or cross-β structure-mediated activity) of the protein present in the circulation. In an even more preferred embodiment the compound capable of increasing cross-β structure formation is glucose. Under certain circumstances the addition of glucose to a protein leads to an irreversible, non-enzymatic glycation reaction in which predominantly a glucose molecule is attached to the free amino groups of lysine residues in a protein. In addition, N-termini and free amino groups of arginine residues are prone to glycation. It is disclosed herein within the experimental part that glycation leads to cross-β structure formation. Hence, the invention provides a method for increasing extracellular protein degradation and/or protein clearance comprising providing a compound capable of increasing cross-β structure formation of the protein present in the circulation.

Other examples of compounds capable of increasing (or mimicking) cross-β structure formation in a protein are apolar solutions, urea (as disclosed herein within the experimental part), ions (for example Zn²⁺). However, it is clear that there are also other ways to increase or mimic cross-β structure formation for example by denaturation, low pH, temperature, mutations or protein modification in general (for example oxidation).

Besides, a method for increasing extracellular protein degradation and/or protein clearance comprising increasing cross-β structure formation of the protein present in the circulation via any of the above described methods to degrade and/or remove, preferably, the protein which comprises the cross-β structure, it is also possible to degrade and/or remove a protein which does not comprise a cross-β structure. This is for example accomplished by providing a compound comprising a cross-β structure and a compound comprising tPA-like activity at or near the protein which needs to be degraded and/or removed. An example of a compound comprising a cross-β structure is fibrin or a fragment thereof comprising the cross-β structure and an example of a compound comprising tPA-like activity is tPA.

In another embodiment the invention provides a method for decreasing extracellular protein degradation and/or protein clearance comprising decreasing cross-β structure formation of the protein present in the circulation. More preferably, the invention provides a method for decreasing extracellular protein degradation and/or protein clearance comprising providing a compound capable of decreasing cross-β structure formation of the protein present in the circulation. Decreasing of cross-β structure formation is for example accomplished by shielding or blocking of the groups involved in the formation of a cross-β structure. Examples of compounds capable of decreasing cross-β structure formation are Congo red, antibodies, β-breakers, phosphonates, heparin, amino-guanidine or laminin.⁴⁵ Yet another way to decrease cross-β structure formation in a protein is by removal of a glucose group involved in the glycation of the protein.

In yet another embodiment the invention provides a method for modulating extracellular protein degradation and/or protein clearance comprising modulating tPA, or tPA-like activity. tPA induces the formation of plasmin through cleavage of plasminogen. Plasmin cleaves fibrin and this occurs during lysis of a blood clot. Activation of plasminogen by tPA is stimulated by fibrin or fibrin fragments, but not by its precursor fibrinogen. The term “tPA-like activity” is herein defined as a compound capable of inducing the formation of plasmin, possibly in different amounts, and/or other tPA mediated activities. Preferably, tPA-like activity is modified such that it has a higher activity or affinity towards its substrate and/or a cofactor. This is for example accomplished by providing the tPA-like activity with multiple binding domains for cross-β structure comprising proteins. Preferably, the tPA-like activity is provided with multiple finger domains. It is herein disclosed that the three-dimensional structures of the tPA finger-domain and the fibronectin finger-domains 4-5 reveals striking structural homology with respect to local charge-density distribution. Both structures contain a similar solvent exposed stretch of five amino-acid residues with alternating charge; for tPA Arg7, Glu9, Arg23, Glu32, Arg30, and for fibronectin Arg83, Glu85, Lys87, Glu89, Arg90, located at the fifth finger domain, respectively. The charged-residue alignments are located at the same side of the finger module. Hence, preferably, the tPA-like activity is provided with one or more extra finger domain(s) which comprise(s) ArgXGlu(X)13Arg(X)8GluXArg or ArgXGluXLysXGluArg.

The activity of tPA and/or the tPA mediated activation of plasminogen is increased by the binding to fibrin fragments, or other protein fragments that have a lysine or an arginine at the carboxy-terminal end. B-type carboxypeptidases, including but not limited to carboxypeptidase B (CpB) or Thrombin Activatable Fibinolysis Inhibitor (TAFI, also named carboxypeptidase U or carboxypeptidase R), are enzymes that cleave off carboxy-terminal lysine and arginine residues of fibrin fragments that would otherwise bind to tPA and/or plasminogen and stimulate plasmin formation.

In a preferred embodiment the invention provides a method for increasing extracellular protein degradation and/or protein clearance comprising providing a compound capable of increasing tPA-like and/or tPA mediated activity or activities. In an even more preferred embodiment the invention provides a method for increasing extracellular protein degradation and/or protein clearance comprising providing a compound capable of increasing tPA-like activity, wherein the compound comprises a cross-β structure. In another embodiment, the invention provides a method for increasing extracellular protein degradation and/or protein clearance comprising providing a compound capable of inhibiting B-type carboxypeptidase activity. In a more preferred embodiment the compound comprises carboxypeptidase inhibitor (CPI) activity.

In yet another embodiment the invention provides a method for decreasing extracellular protein degradation and/or protein clearance comprising providing a compound capable of decreasing tPA-like activity. More preferably, the invention provides a method for decreasing extracellular protein degradation and/or protein clearance comprising providing a compound capable of decreasing tPA-like activity or tPA-mediated activity or activities, wherein the compound is a protein and/or a functional equivalent and/or a fimctional fragment thereof. For example, such a compound capable of decreasing tPA-like activity is an inhibitor of tPA or a substrate of tPA which binds and does not let go. Examples of a compound capable of decreasing tPA-like activity or tPA-mediated activity include but are not limited to, lysine, arginine, e-amino-caproic acid or tranexamic acid, serpins (for example neuroserpin, PAI-1), tPA-Pevabloc, antibodies that inhibit tPA-like activity or tPA-mediated activity or B-type carboxypeptidase(s). For example, providing lysine results in the prevention or inhibition of binding of a protein comprisiung a C-terminal lysine-residue to the Kringle domain of plasminogen. Hence, tPA activation is prevented or inhibited. Preferably, the compound capable of decreasing tPA-like activity or tPA-mediated activity or activities reduce the tPA-like activity or tPA-mediated activity or activities and even more preferably, the tPA-like activity or tPA-mediated activity or activities is completely inhibited.

A functional fragment and/or a functional equivalent is typically defined as a fragment and/or a equivalent capable of performing the same function, possibly in different amounts. For example, a functional fragment of an antibody capable of binding to a cross-β structure would be the Fab' fragment of the antibody.

In yet another embodiment the invention provides a method for modulating extracellular protein degradation and/or protein clearance comprising modulating an interaction between a compound comprising a cross-β structure and a compound comprising tPA-like activity. In another embodiment the invention provides a method for decreasing extracellular protein degradation and/or protein clearance comprising decreasing an interaction between a compound comprising a cross-β structure and a compound comprising tPA-like activity. Such a compound is for example a chemical, a proteinaceous substance or a combination thereof. In a more preferred embodiment the invention provides a method for decreasing extracellular protein degradation and/or protein clearance comprising providing a compound capable of decreasing an interaction between a compound comprising a cross-β structure and a compound comprising tPA-like activity. Even more preferably, the invention provides a method for decreasing extracellular protein degradation and/or protein clearance according to the invention, wherein the compound is a protein and/or a functional equivalent and/or a functional fragment thereof. Even more preferably, the protein is an antibody and/or a functional equivalent and/or a functional fragment thereof. Other examples are Congo red or Thioflavin. The invention also provides a method for decreasing extracellular protein degradation and/or protein clearance comprising decreasing an interaction between a compound comprising a cross-β structure and a compound comprising tPA-like activity, wherein the interaction is decreased by providing a compound capable of competing with the interaction. More in particular, the compound capable of competing with the interaction comprises a finger domain and even more preferably, the finger domain comprises a stretch of at least 5 amino acid residues with alternating charge, for example ArgXGlu(X)₁₃Arg(X)₈GluXArg or ArgXGluXLysXGluArg. Preferably, the compound is fibronectin, FXII, HGFa or tPA. It is clear that the invention also comprises a method for increasing extracellular protein degradation and/or protein clearance comprising increasing an interaction between a compound comprising a cross-β structure and a compound comprising tPA-like activity. This is for example accomplished by providing a compound capable of increasing an interaction between a compound comprising a cross-pstructure and a compound comprising tPA-like activity. Preferably, the compound capable of increasing an interaction between a compound comprising a cross-β structure and a compound comprising tPA-like activity is a protein and/or a functional equivalent and/or a finctional fragment thereof. For example an antibody which stabilizes the interaction between a compound comprising cross-β structure and a compound comprising tPA-like activity, rendering the tPA-like activity in a continuous activated state, hence protein degradation and/or protein clearance is increased. However it is appreciated that increasing an interaction between a compound comprising a cross-β structure and a compound comprising tPA-like activity is also accomplished by mutations in either the compound comprising a cross-β structure or in the compound comprising tPA-like activity, like swapping of domains (for example by providing the compound comprising tPA-like activity with other or more finger domains (obtainable from tPA, fibronectin, FXII or HGFa) or by including binding domains of for example RAGE or CD36.

In yet another embodiment the invention provides a method for modulating extracellular protein degradation and/or protein clearance comprising modulating an interaction of a compound comprising tPA-like activity and the substrate of the activity. It is clear that there are multiple ways by which the interaction can either be increased or decreased. An increase in the interaction between a compound comprising tPA-like activity and the substrate of the activity is for example accomplished by providing the compound comprising tPA-like activity with a mutation or mutations which improve the affinity of the compound with tPA-like activity for its substrate.

In yet another embodiment the invention provides a method for removing cross-β structures from the circulation, using a compound comprising a cross-β structure binding domain. Preferably, the compound is tPA or the finger domain of tPA. It is clear that the invention also comprises other cross-β structure binding domains, including, but not limited to the finger domains of HGFa, FXII and fibronectin. It is clear that the invention also comprises antibodies that bind cross-β structures.

The present invention further discloses the use of a novel strategy to prevent the formation or to decrease/diminish (amyloid) plaques involved in a conformational disease, type II diabetes and/or ageing (e.g., Alzheimer's disease). Plaques are typically defined as extracellular fibrillar protein deposits (fibrillar aggregates) and are characteristic of degenerative diseases. The “native” properties of the constituent amyloid proteins may vary: some are soluble oligomers in vivo (e.g., transthyretin in familial amyloid polyneuropathy), whereas others are flexible peptides (e.g., amyloid-b in Alzheimer's disease (AD)). The basic pathogenesis of conformational diseases, for example neurodegenerative disorders (AD, prion disorders) is thought to be related to abnormal pathologic protein conformation, i.e the conversion of a normal cellular and/or circulating protein into an insoluble, aggregated, β-structure rich form which is deposited in the brain. These deposits are toxic and produce neuronal dysfunction and death. The formation of cross-β structures has thus far only been associated with severe pathological disorders. Our results, show that tPA and other receptors for cross-β structure forming proteins can bind denatured proteins, indicating that a large number of proteins are capable of adopting a conformation containing cross-β or cross-β-like structures. Taken together, the formation of a cross-β structure initiates or participates in a physiological cascade of events; necessary to adequately deal with removal of unwanted molecules, i.e., misfolded proteins, apoptotic cells or even pathogens. By increasing cross-β structure formation in a protein involved in a conformational disease, the pathway for protein degradation and/or protein clearance is activated and the protein is degraded, resulting in a decreasing plaque or more preferably, the plaque is completely removed. Hence, the effects of the conformational disease are diminished or more preferably, completely abolished.

In a further embodiment the invention provides the use of a compound capable of increasing cross-β structure formation for diminishing plaques involved in a conformational disease. In another embodiment the invention provides the use of a compound capable of binding to a cross-β structure for diminishing plaques and/or inhibiting cross-β structure mediated toxicity involved in a conformational disease. In a preferable use of the invention, the compound is a protein and/or a functional equivalent and/or a functional fragment thereof and even more preferably, the protein is tPA, a finger domain, an antibody and/or a functional equivalent and/or a functional fragment thereof. Examples of such antibodies are 4B5 or 3H7.

In yet a further embodiment the invention provides use of a compound capable of increasing tPA-like activity for diminishing plaques involved in a conformational disease. Preferably, the tPA-like activity is modified such that it has a higher activity or affinity towards its substrate and/or cofactor. This is for example accomplished by providing the tPA-like activity with multiple binding domains for cross-β structure comprising proteins. Preferably, the binding domain comprises a finger domain and even more preferably, the finger domain comprises a stretch of at least 5 amino acid residues with alternating charge, for example ArgXGlu(X)₁₃Arg(X)₈GluXArg or ArgXGluXLysXGluArg. Even more preferably, the finger domain is derived from fibronectin, FXII, HGFa or tPA.

In yet another embodiment the invention provides the use of a compound capable of binding to a cross-β structure for the removal of cross-β structures. Preferably, the compound is a protein and/or a functional equivalent and/or a functional fragment thereof. More preferably, the compound comprises tPA or tPA-like activity and/or a functional equivalent and/or a functional fragment thereof. Even more preferably, the functional fragment comprises a finger domain. Preferably, the finger domain comprises a stretch of at least 5 amino acid residues with alternating charge, for example ArgXGlu(X)₁₃Arg(X)₈GluXArg or ArgXGluXLysXGluArg. Even more preferably, the finger domain is derived from fibronectin, FXII, HGFa or tPA. In another preferred embodiment the protein is an antibody and/or a functional equivalent and/or a functional fragment thereof. With this use the invention provides for example a therapeutic method to remove cross-β structure comprising proteins from for example the circulation, preferably via extracorporeal dialysis. For example, a patient with sepsis is subjected to such use by dialysis of blood of the patient through means which are provided with for example, preferably immobilized, finger domains. One could for example couple the finger domains to a carrier or to the inside of the tubes used for the dialysis. In this way, all cross-β structure comprising proteins will be removed from the blood stream of the patient, thereby relieving the patients of the negative effects caused by the cross-β structure comprising proteins. Besides finger domain comprising compounds, it is also possible to use other cross-β structure binding compounds, like antibodies or Congo Red. It is also clear that the use could be applied in haemodialysis of kidney patients.

In yet another embodiment the invention provides the use of a compound capable of increasing or stabilising an interaction of a compound comprising a cross-β structure and a compound comprising tPA-like activity for diminishing plaques involved in a conformational disease. Examples of a compound capable of increasing or stabilising an interaction of a compound comprising a cross-β structure and a compound comprising tPA-like activity are given herein. A preferable use according to the invention is provided, wherein the conformational disease is Alzheimer or diabetes. It is clear that the invention not only provides a use to decrease/diminish plaques involved in a conformational disease but that the onset of the disease can also be inhibited or more preferably, completely prevented. Examples of diseases which can be prevented and/or treated according to the invention are conformational disease, amyloidosis type diseases, atherosclerosis, diabetes, bleeding, thrombosis, cancer, sepsis and other inflammatory diseases, Multiple Sclerosis, auto-immune diseases, disease associated with loss of memory or Parkinson and other neuronal diseases (epilepsy).

In another embodiment the invention provides the use of an antibody capable of recognizing a cross-β structure epitope for determining the presence of plaque involved in a conformational disease. In yet another embodiment the invention provides use of a cross-β structure binding domain (preferably, a finger domain from for example tPA) for determining the presence of a plaque involved in a conformational disease.

These uses of the invention provide a new diagnostic tool. It was not until the present invention that a universal b-structure epitope was disclosed and that a diagnostic assay could be based on the presence of the cross-β structure. Such use is particular useful for diagnostic identification of conformational diseases or diseases associated with amyloid formation, like Alzheimer or diabetes. It is clear that this diagnostic use is also useful for other diseases which involve cross-β structure formation, like all amyloidosis type diseases, atherosclerosis, diabetes, bleeding, cancer, sepsis and other inflammatory diseases, Multiple Sclerosis, auto-immune diseases, disease associated with loss of memory or Parkinson and other neuronal diseases (epilepsy). For example, one can use a finger domain (of for example tPA) and provide it with a label (radio active, fluorescent etc.). This labeled finger domain can then be used either in vitro or in vivo for the detection of cross-β structure comprising proteins, hence for determining the presence of a plaque involved in a conformational disease. One can for example use an ELISA assay to determine the amount of sepsis in a patient or one can localize a plaque involved in a conformational disease.

In yet another embodiment the invention provides a recombinant tPA comprising an improved cross-β structure binding domain or multiple cross-β structure binding domains. Preferably, the tPA is provided with multiple, possibly different, finger domains. A recombinant tPA comprising an improved cross-β structure binding domain or multiple cross-β structure binding domains is used for different purposes. For example in a method for the improved treatment of thrombolysis or for the removal of cross-β structure comprising proteins from the circulation of a patient in need thereof. Another use of a recombinant tPA comprising an improved cross-β structure binding domain or multiple cross-β structure binding domains is in diagnostic assays, for example in a BSE detection kit or in imaging experiments. This imaging with a recombinant tPA comprising an improved cross-β structure binding domain or multiple cross-β structure binding domains is for example useful for detection of apoptosis. For example, labelled tPA, for example but not limited to radio-labelled tPA, is inoculated in an individual, followed by detection and localization of the labelled tPA in the body. It is clear that the recombinant tPA comprising a cross-β structure binding domain or multiple cross-β structure binding domains are also useful in therapeutic applications.

Because this invention has made clear that the cross-β structure is harmful when present in certain parts of the body, like for example the brain, and the damage is effected by the combination of cross-β structures with tPA, a method is provided to inhibit cross-β structure-mediated effects comprising providing an effective amount of a protein comprising a finger domain to block the binding sites of the cross-β structure for tPA. The cross-β structure-mediated effects may even be further diminished comprising providing an effective amount of B-type carboxypeptidase activity to inhibit the tPA activity.

In another embodiment, the local cross-β structure-mediated effect can be used against tumors. To that effect, cross-β structure-mediated effects are locally induced to increase local cytotoxicity and/or fibrinolysis comprising locally administering an effective amount of cross-β structures and/or cross-β structure inducing compounds in conjunction with tPA or a compound with tPA-like activity and/or CPI or a compound with CPI-like activity.

The present invention provides, in a further embodiment a method according to to the invention which is carried out ex vivo, e.g., by dialysis. According to this embodiment the circulating fluid (blood) of a subject is brought in a system outside the body for clearing cross-β structures from the circulation. Preferably, such a system is a flow through system, connected to the body circulation with an inlet and an outlet. The cross-β structures are cleared by binding to a cross-β binding compound as defined herein before. It is very important that no elements, such as the cross-β binding compounds from the system are brought into the subject's circulation. Preferred systems are dialysis systems, for that reason among others. The invention further provides devices for carrying out methods as disclosed above. Thus the invention provides a separation device for carrying out a method according to the invention, whereby the apparatus comprises a system for transporting circulation fluids ex vivo, the system provided with means for connecting to a subject's circulation for entry into the system and return from the system to the subject's circulation, the system comprising a solid phase, the solid phase comprising at least one compound capable of binding cross-β structures. Compounds for binding cross-β structures have been disclosed herein. The preferred device is a dialysis apparatus.

The invention also provides for detection of cross-β structures in samples. Such samples may be tissue samples, biopsies and the like, body fluid sample, such as blood, serum, liquor, CSF, urine, and the like. The invention thus provides a method for detecting cross-β structures in a sample, comprising contacting the sample with a compound capable of binding cross-β structures, allowing for binding of cross-β structures to the compound and detecting the complex formed through binding.

Cross-β binding compounds have been defined herein before. Detection of the complex or one of its constituents can be done through any conventional means involving antibodies or other specific binding compounds, further cross-β binding compounds, etc. Detection can be direct, by labelling the complex or a binding partner for the complex or its constituents, or even by measuring a change in a physical or chemical parameter of the complex versus unbound material. It may also be indirect by further binding compounds provided with a label. A label may be a radioactive label, an enzyme, a fluorescent molecule, etc.

The invention further provides devices for carrying out the diagnostic methods. Thus the invention provides a diagnostic device for carrying out a method according to the invention, comprising a sample container, a means for contacting the sample with a cross-β binding compound, a cross-β binding compound and a means for detecting bound cross-β structures. Preferably, the device comprises a means for separating unbound cross-β structures from bound cross-β structures. This can be typically done by providing the cross-β binding compounds on a solid phase.

### DESCRIPTION OF THE FIGURES

FIG. 1. Schematic representation of the “cross-β structure pathway.” The cross-β structure is found in a number of proteins (1). The formation of a cross-β structure can be triggered by several physiological or pathological conditions and subsequently initiates a cascade of events, the “cross-β structure pathway.” Among the factors that trigger or regulate the formation of a cross-β structure within a given protein are: 1) the physicochemical properties of the protein, 2) proteolysis, 3) regulated posttranslational modification, including cross-linking, oxidation, phosphorylation, glycosylation and glycation, 4) glucose, and 5) zinc. Certain mutations within the sequence of a protein are known to increase the ability of the protein to adopt a cross-β structure and form amyloid fibrils. These mutations are often found in hereditary forms of amyloidosis, for example in AD. The present invention discloses multiple novel examples of proteins capable of adopting a cross-β structure.

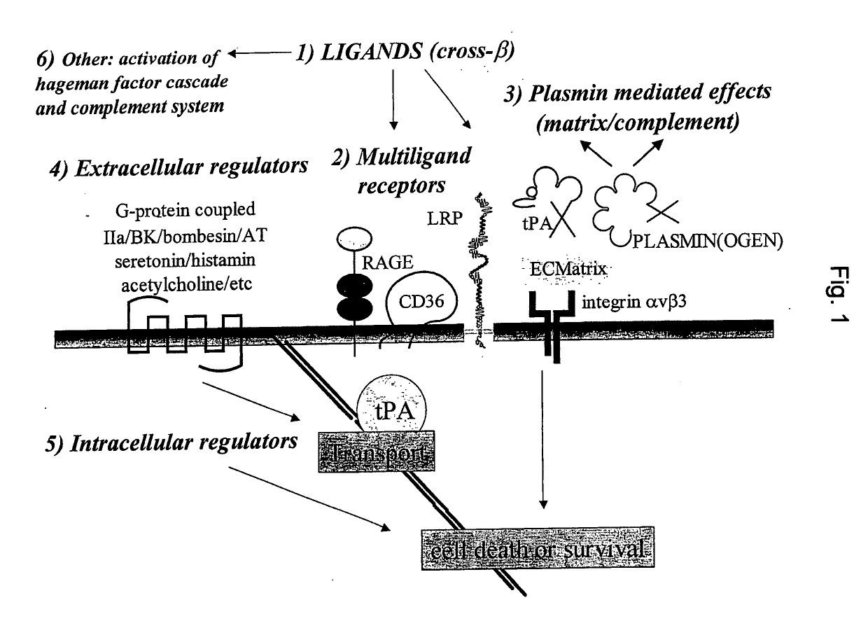

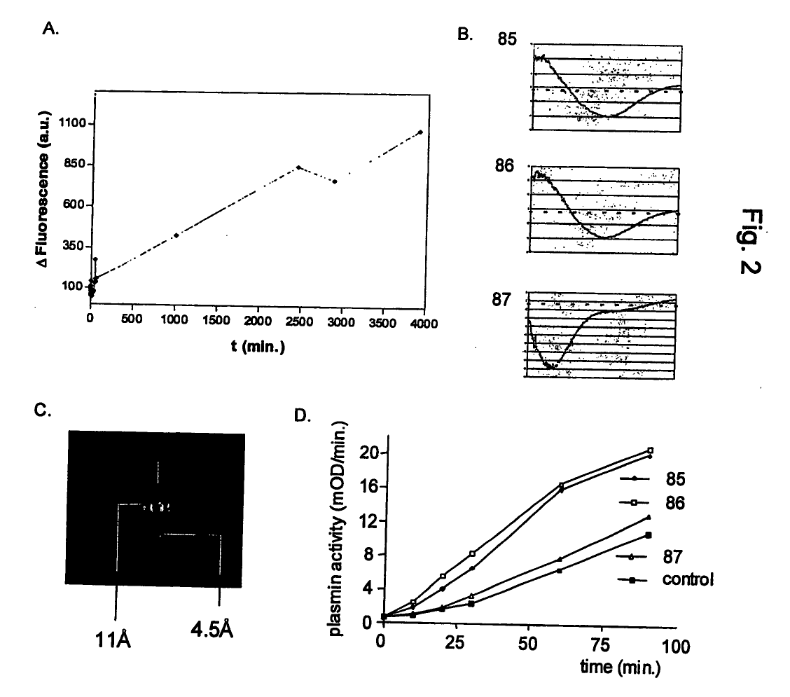

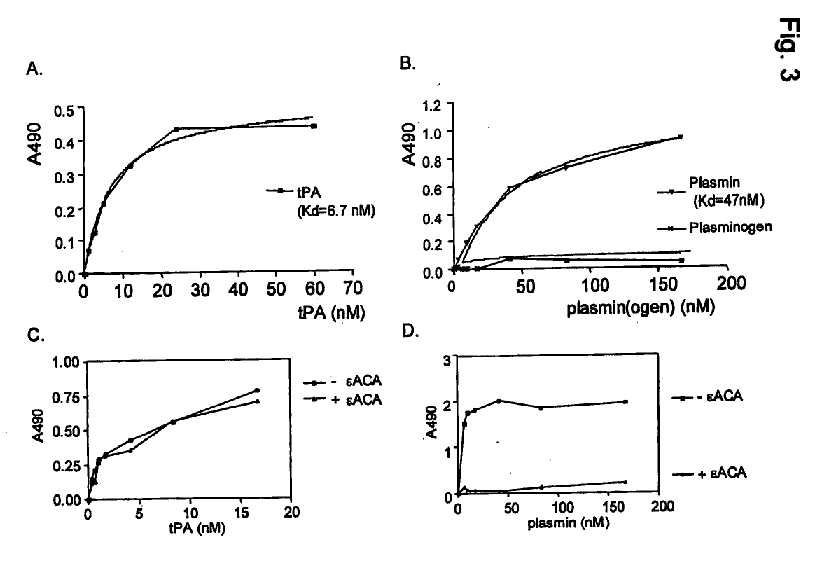

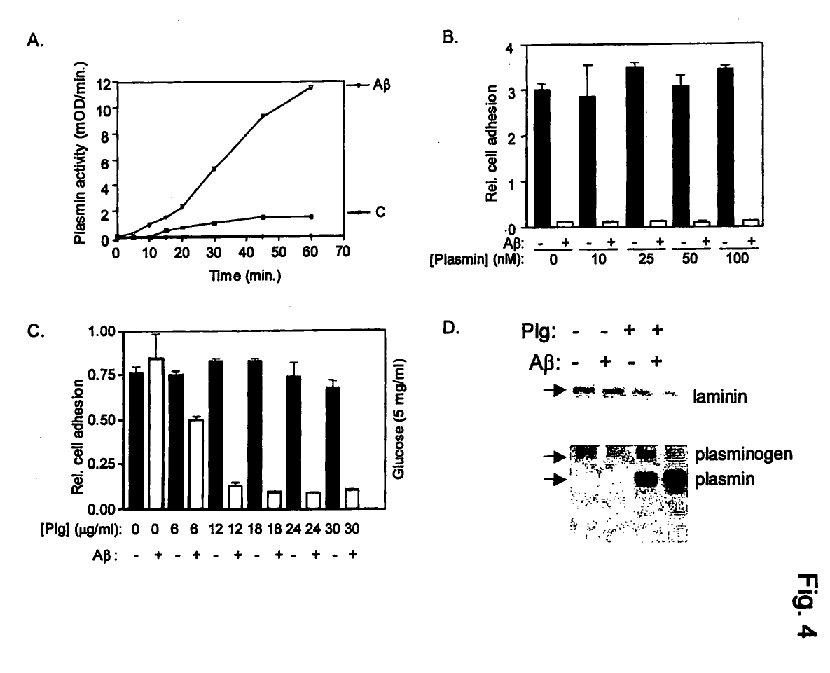

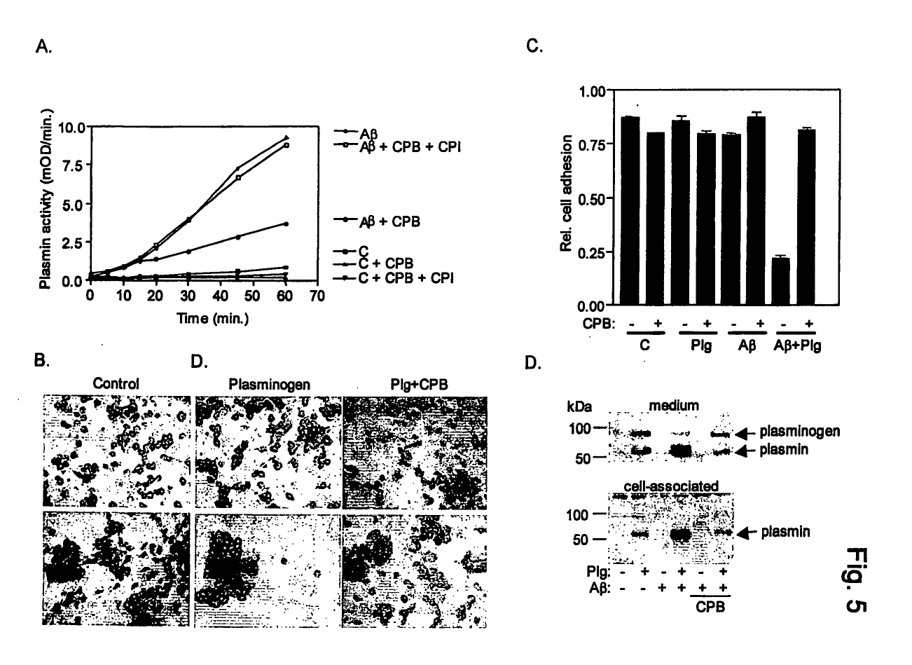

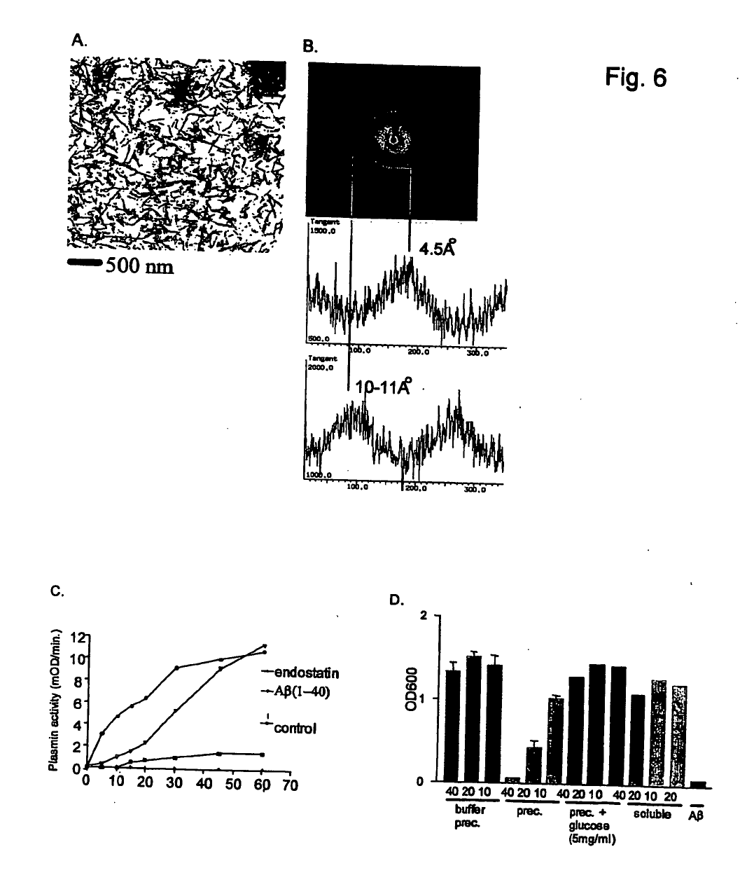

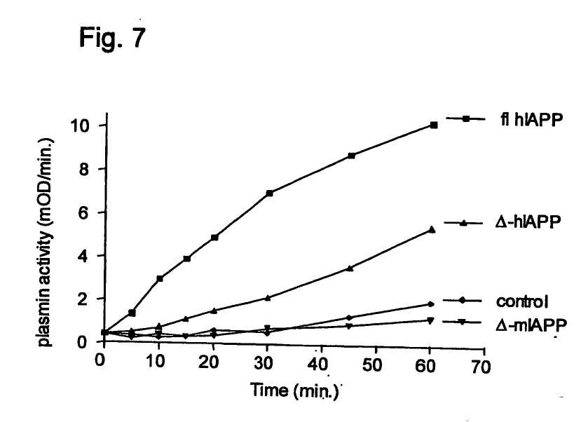

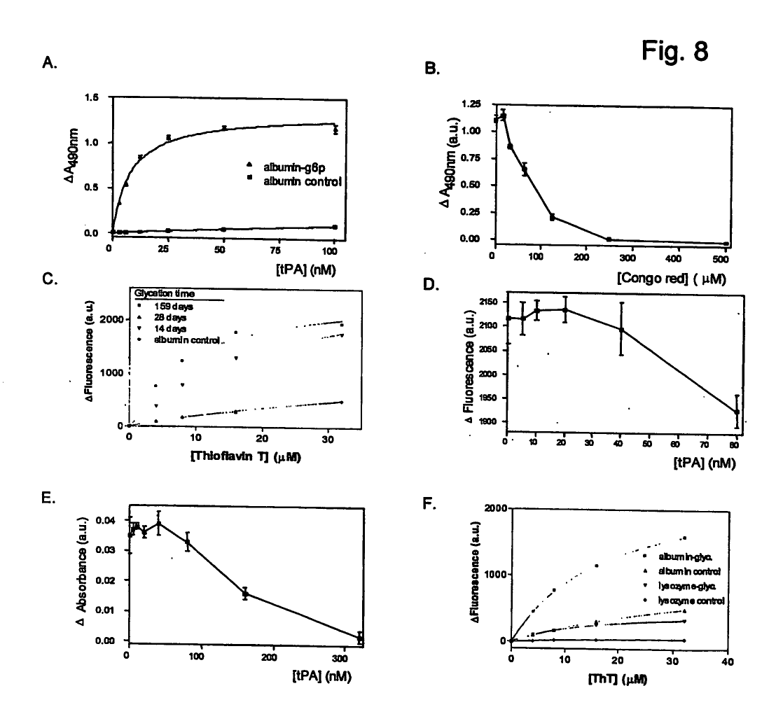

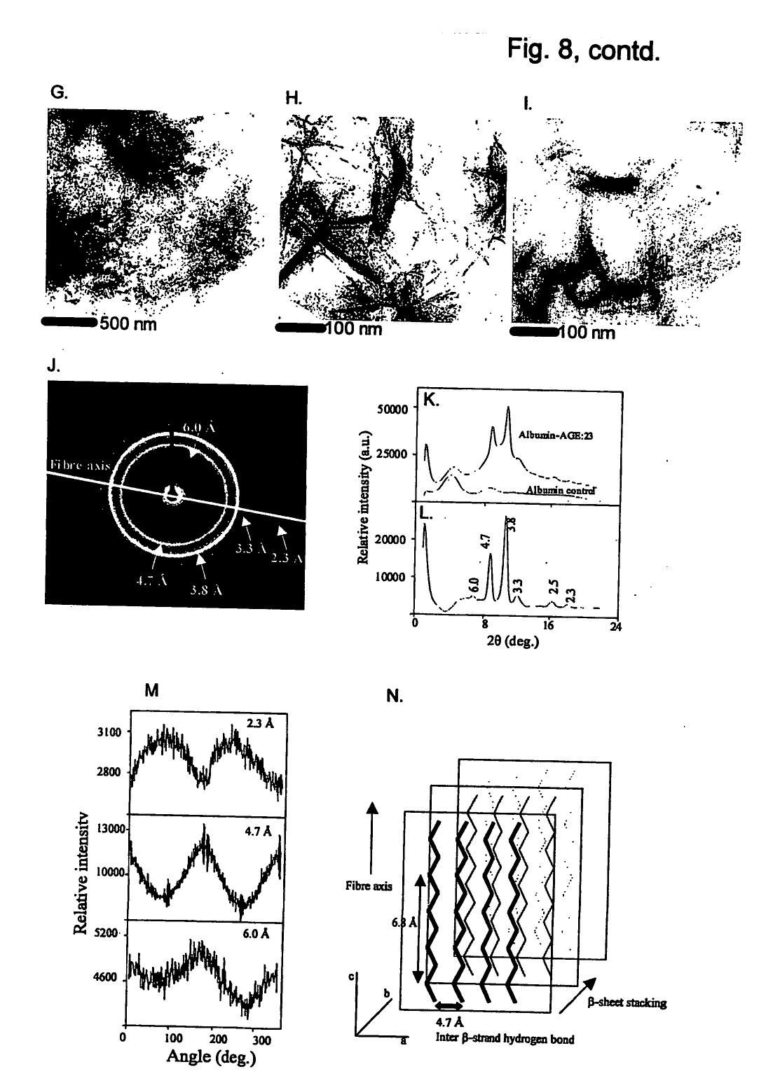

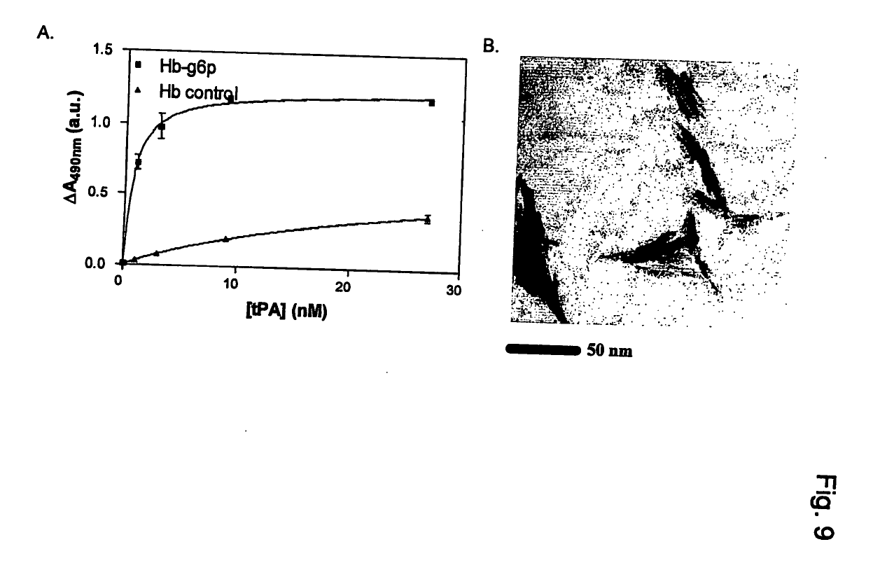

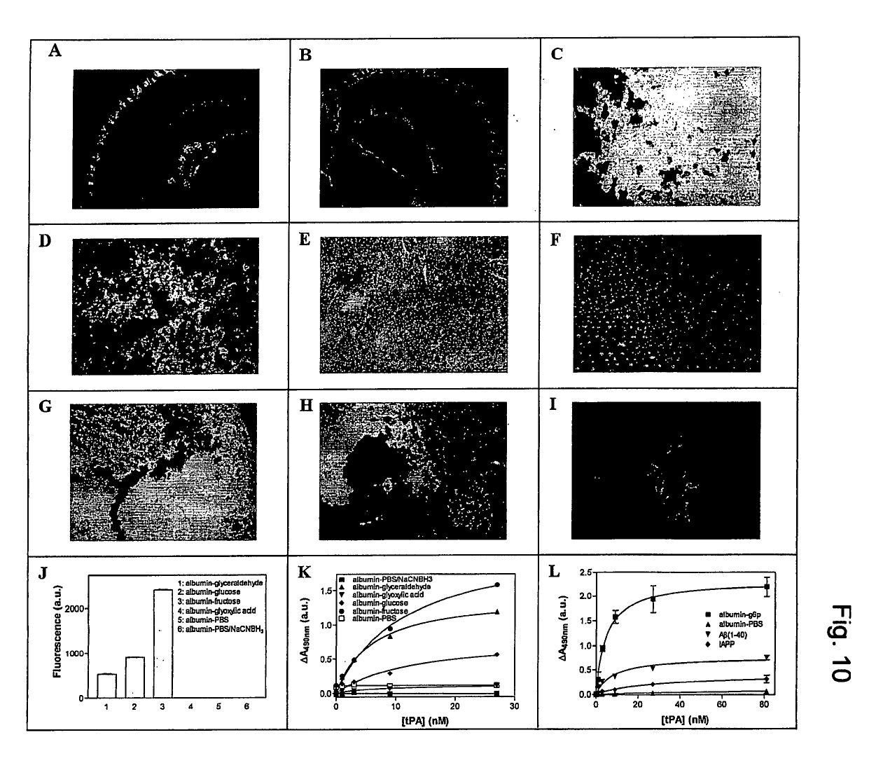

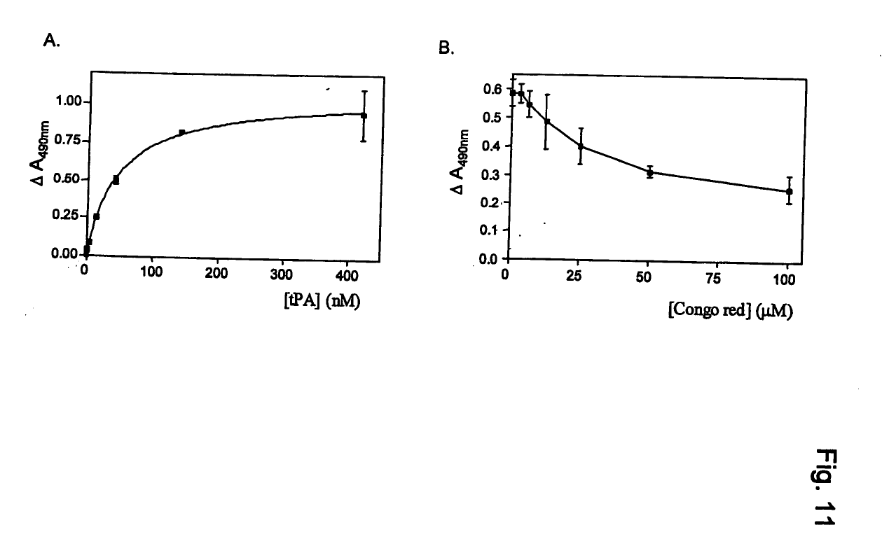

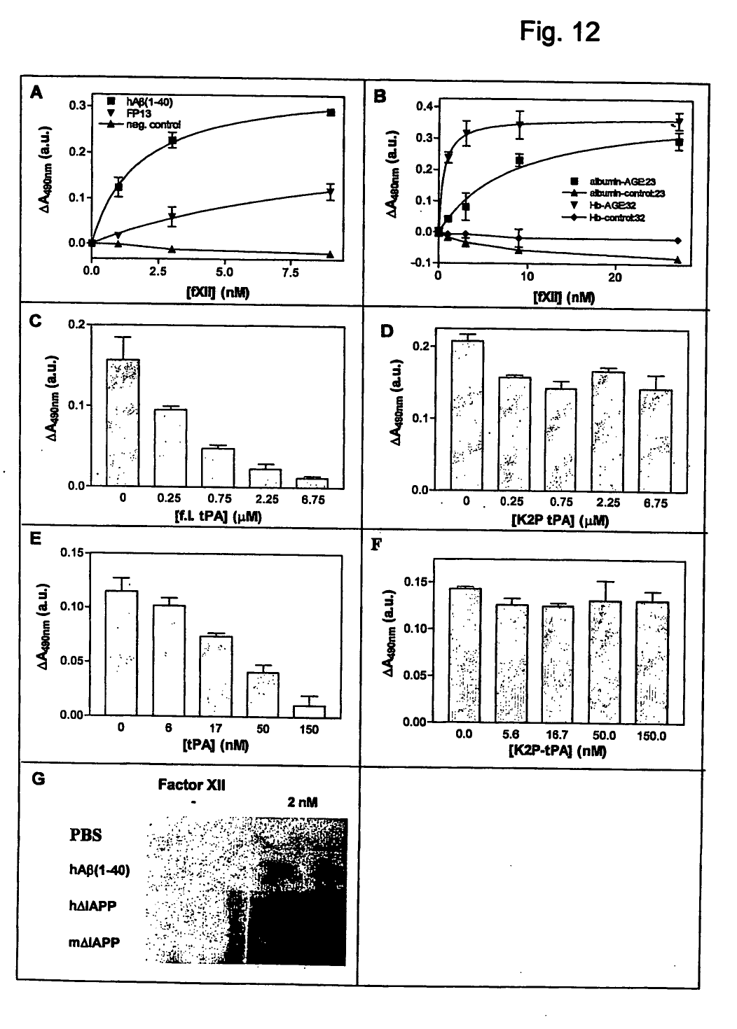

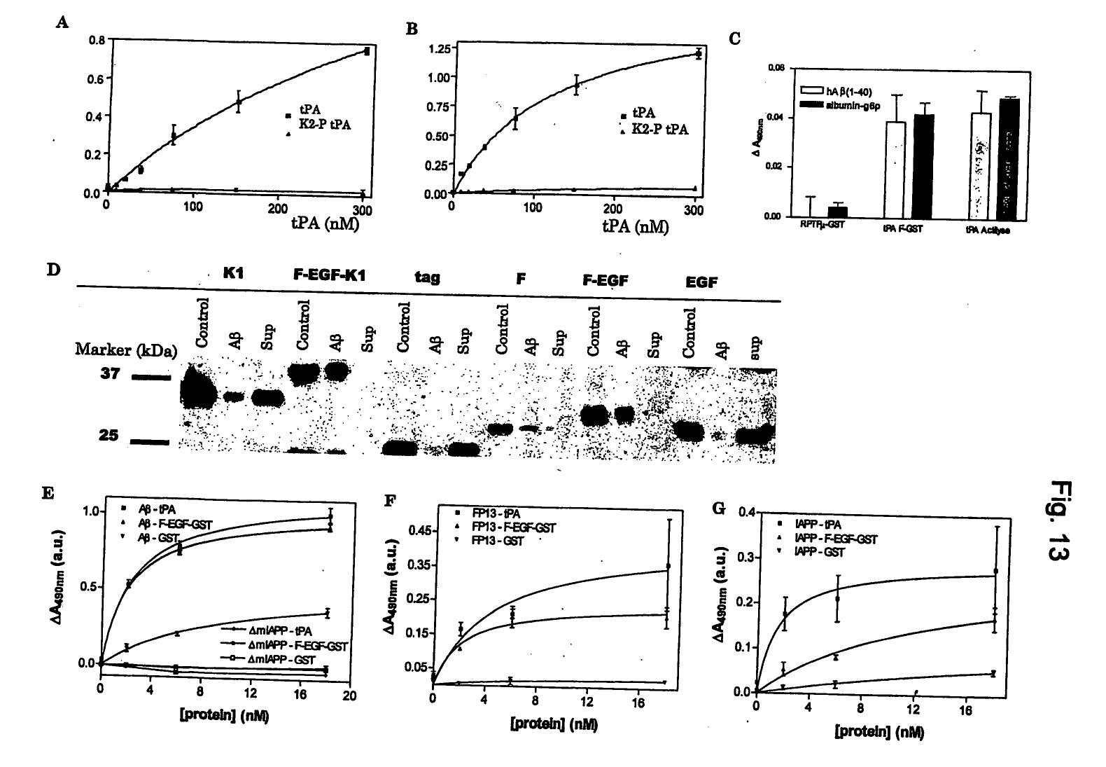

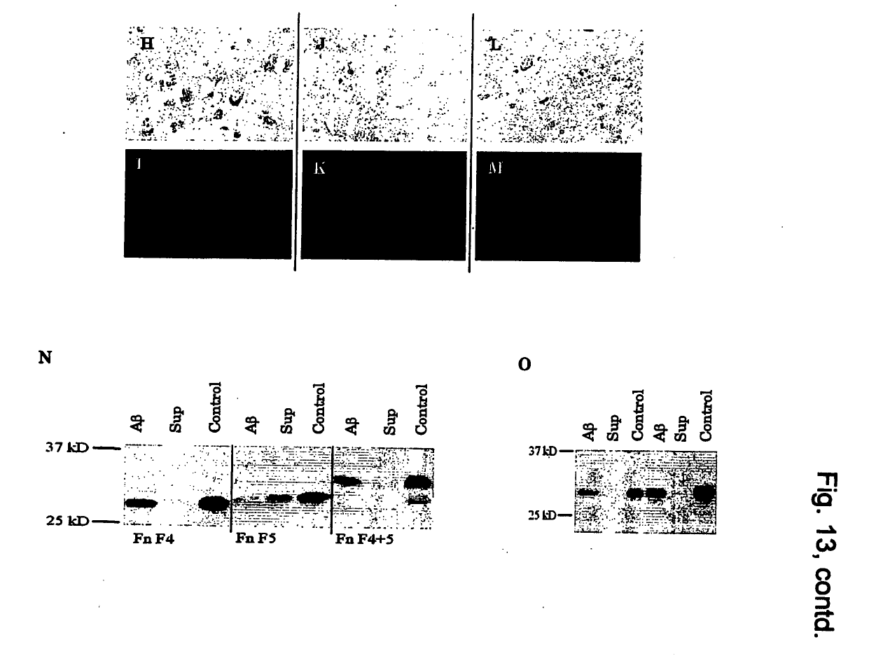

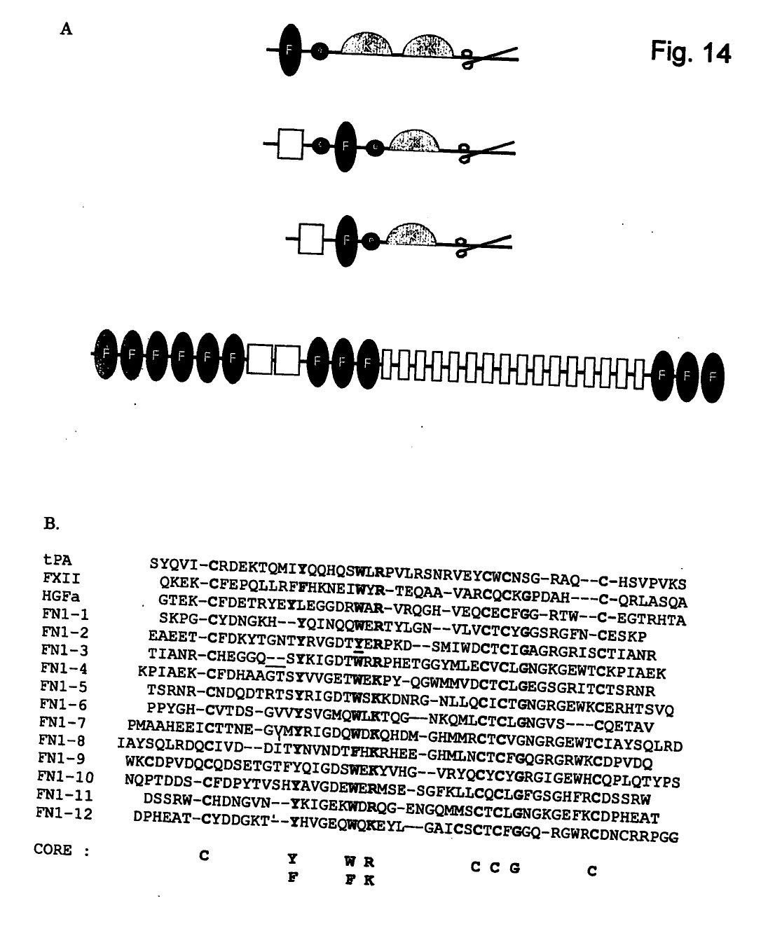

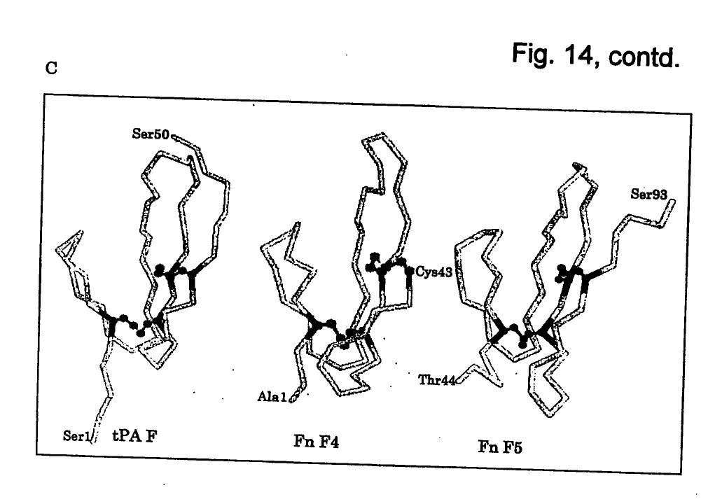

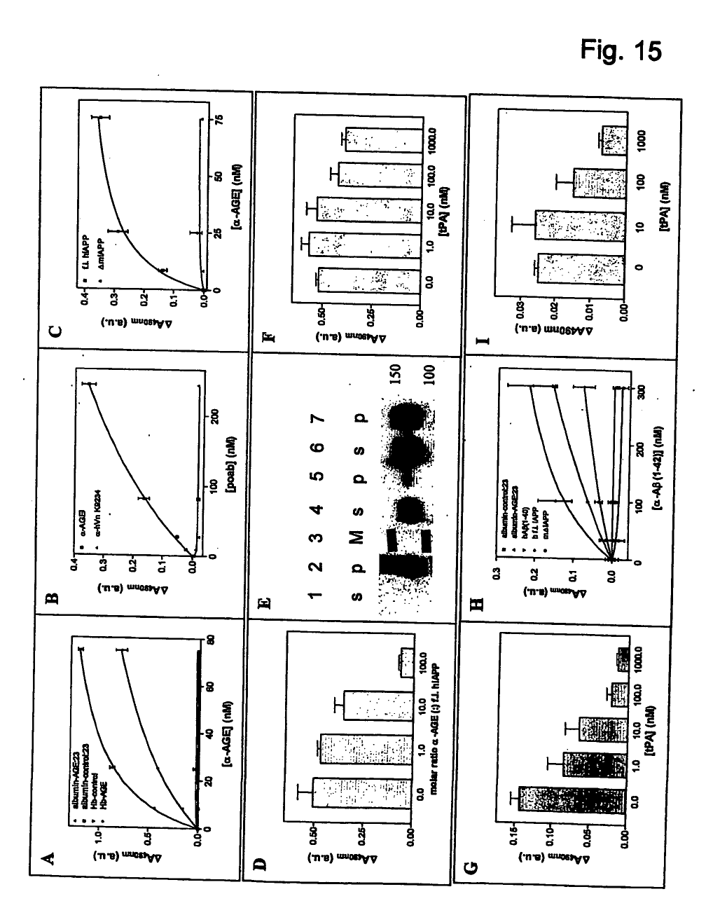

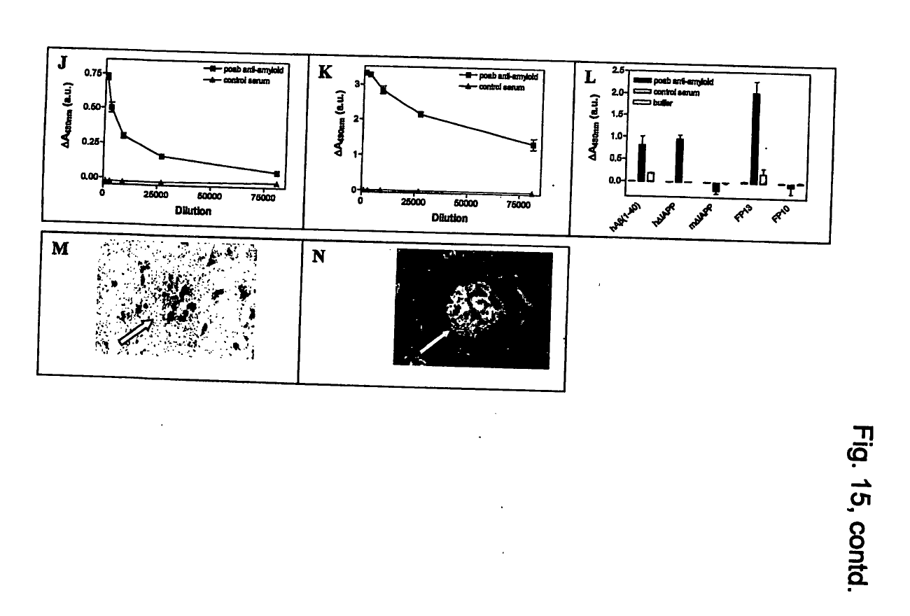

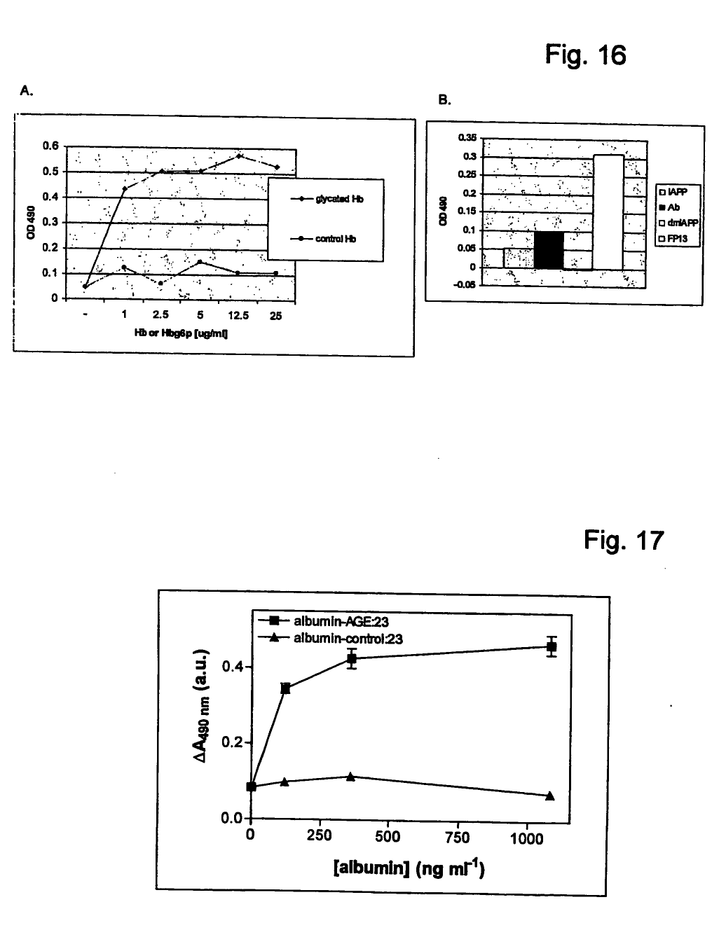

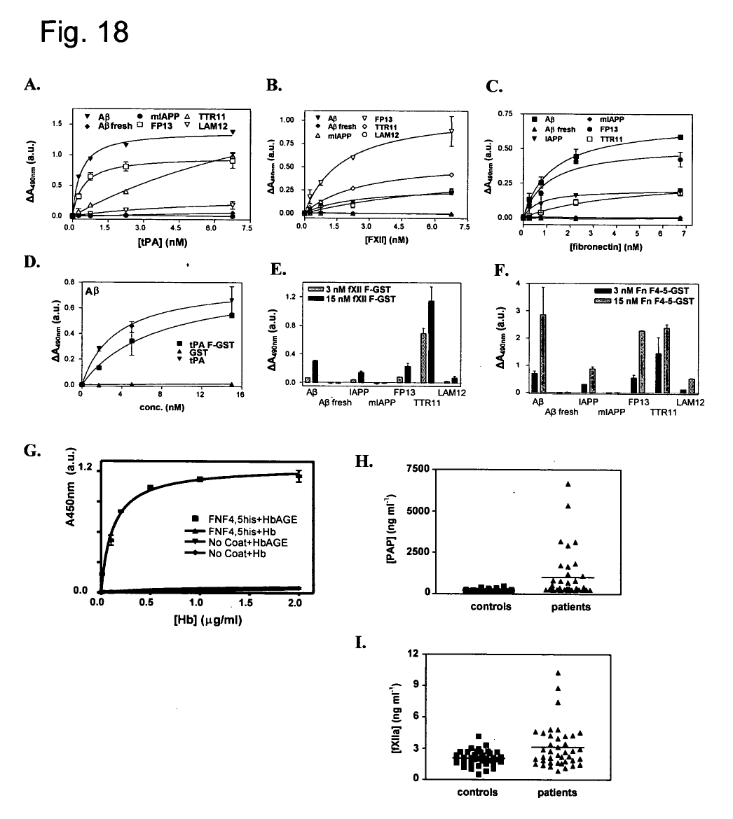

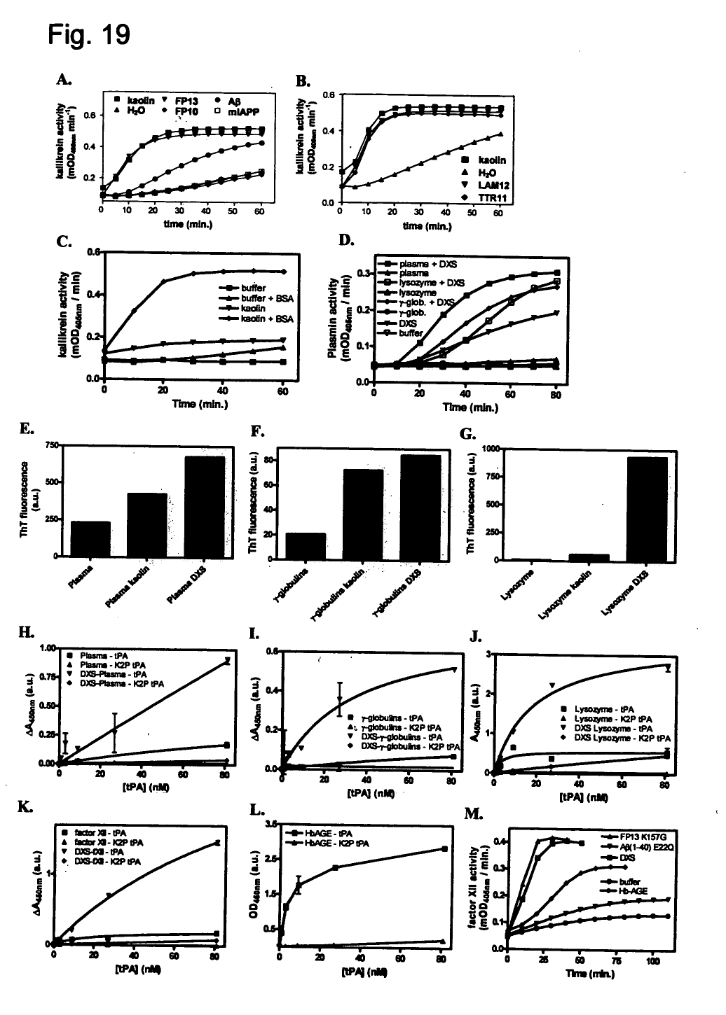

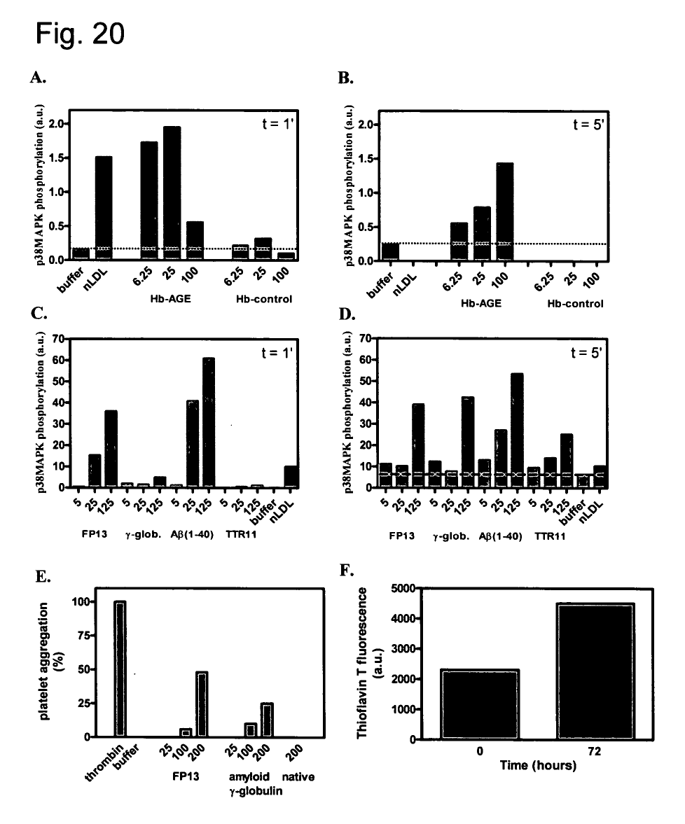

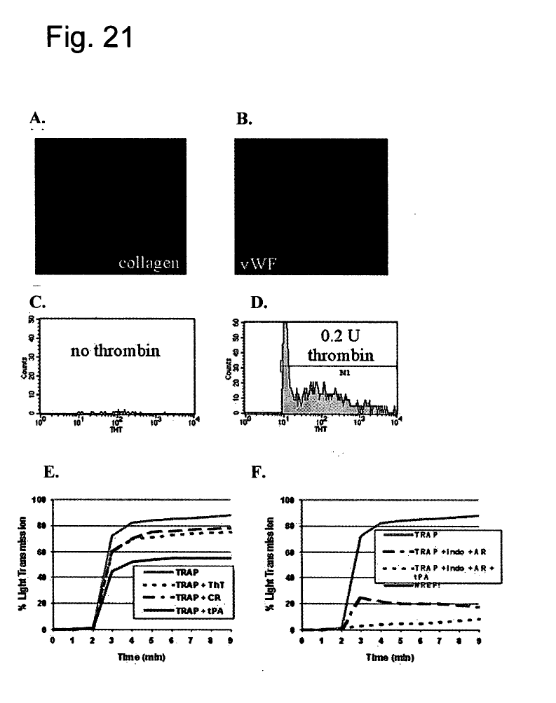

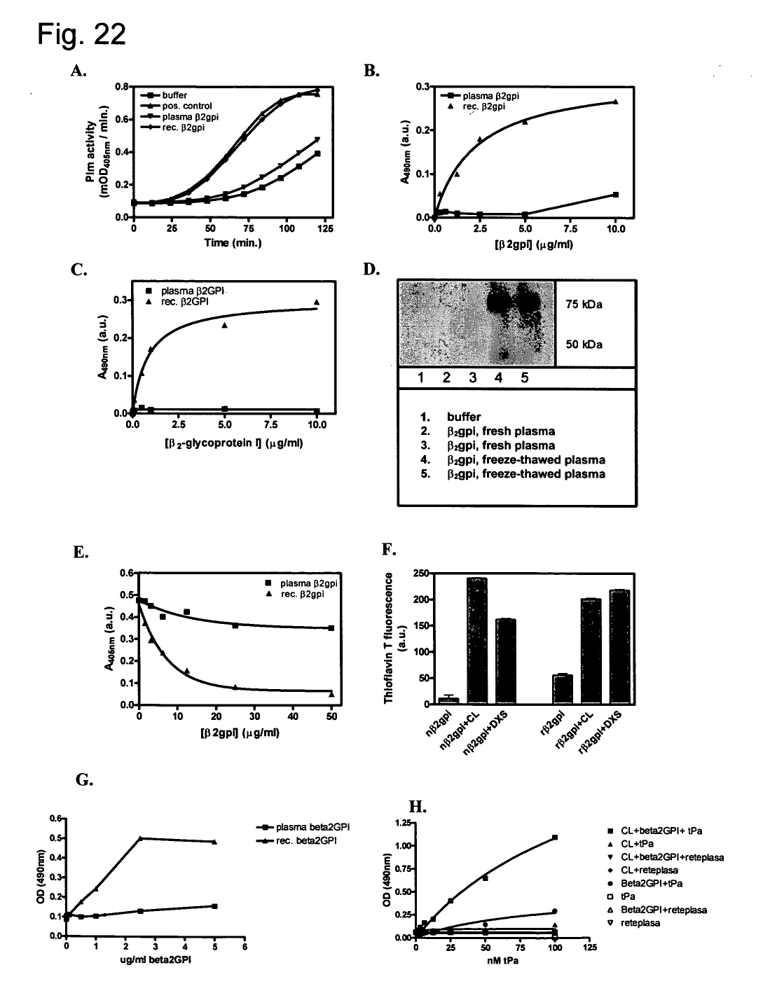

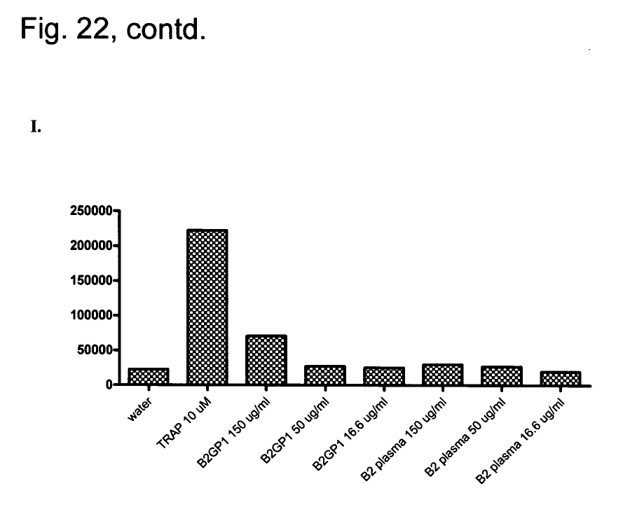

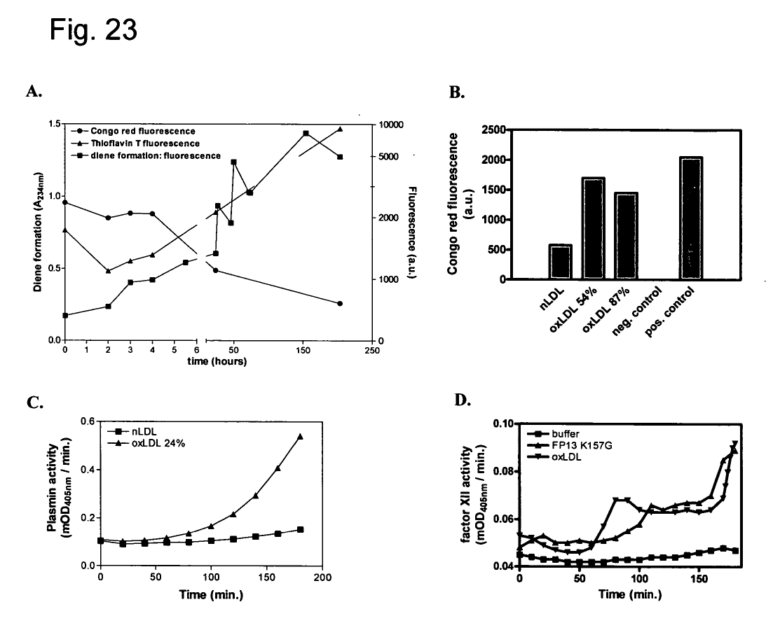

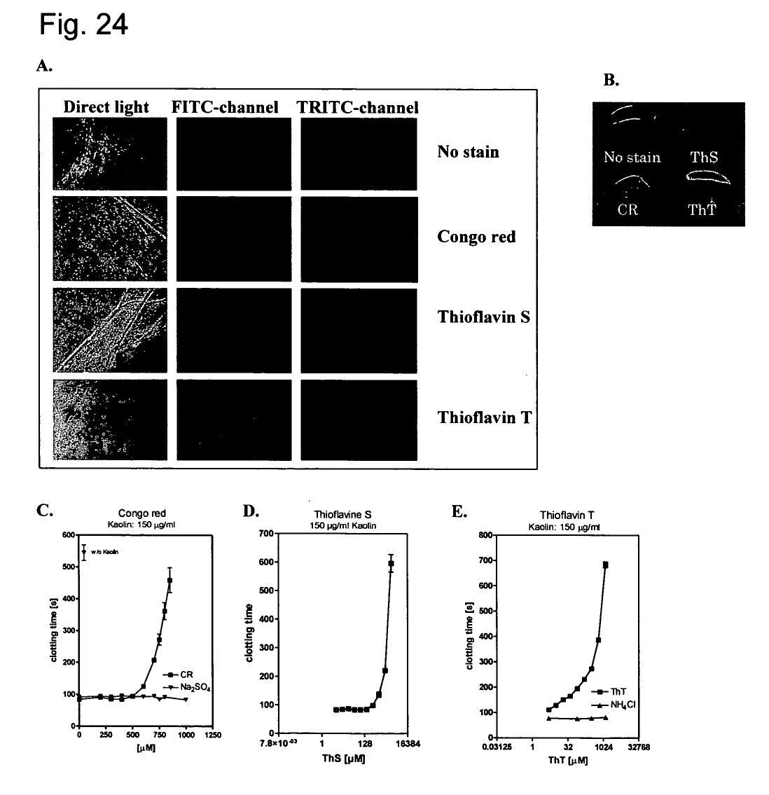

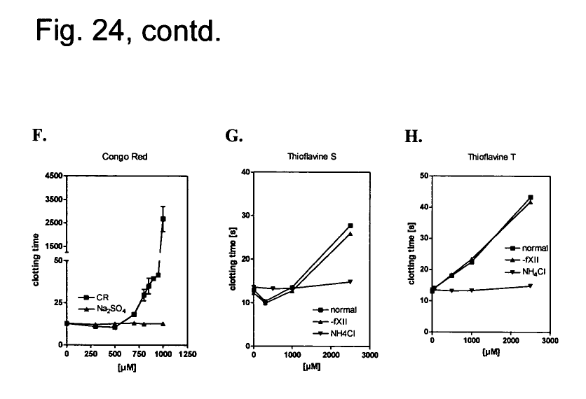

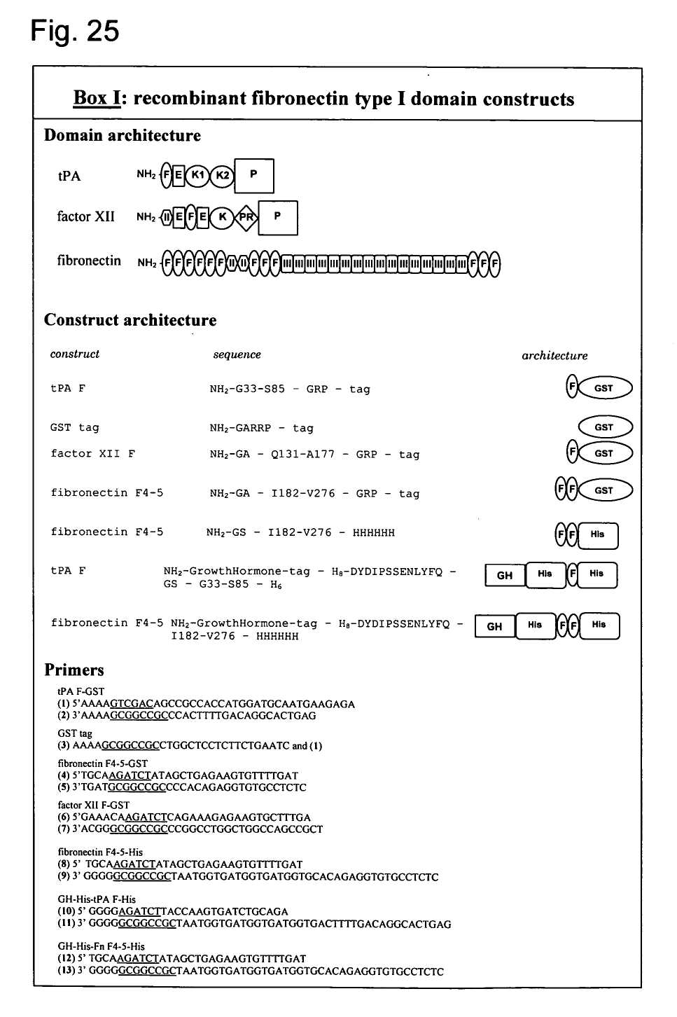

Several proteins are known to bind cross-β containing proteins (2). These proteins are part of the, herein disclosed, signalling cascade (“cross-β structure pathway”) that is triggered upon formation of a cross-β structure. The “cross-β structure pathway” is modulated in many ways (3,4,5). Factors that regulate the pathway include modulators of synthesis and secretion, including NO regulators, as well as modulators of activity, including protease inhibitors. The pathway is involved in many physiological and pathological processes, including but not limited to atherosclerosis, diabetes, amyloidosis, bleeding, inflammation, multiple sclerosis, Parkinson's disease, sepsis, haemolytic uremic syndrome (7). Given the established role for tPA in long term potentiation the “cross-β structure pathway” may also be involved in learning.

FIG. 2. Cross-β structure in fibrin. (A) Thioflavin T fluorescence of a fibrin clot. A fibrin clot was formed in the presence of Thioflavin T and fluorescence was recorded at indicated time points. Background fluorescence of buffer, Thioflavin T and a clot formed in the absence of Thioflavin T, was substracted. (B) Circular dichroism analysis of fibrin derived peptide 85, 86 and 87. Ellipticity (Dg.cm²/dmol) is plotted against wavelength (nm). The CD spectra demonstrate that peptide 85 and 86, but not peptide 87 contain β-sheets. (C) X-ray fibre diffraction analysis of peptide 85 reveals that the peptide forms cross-β sheets. (D) Plasminogen activation assay with fibrin peptides 85, 86 and 87. It is seen that peptide 85 and 86, both containing a cross-β structure do stimulate the formation of plasmin by tPA, whereas peptide 87, which lacks a cross-β structure does not.

FIG. 3. Binding of tPA, plasminogen and plasmin to Aβ. Aβ was coated onto plastic 96 well plates. Increasing concentrations of either (A) tPA or (B) plasmin(ogen) were allowed to bind to the immobilized peptide. After extensive washing tPA and plasmin(ogen) binding was assessed by enzyme-linked immunosorbent assays using anti-tPA and anti-plasminogen antibodies. Binding of (C) tPA and (D) plasrin to Aβ in the presence of 50 mM ε-aminocaproic acid (ε-ACA) was assessed as in A and B.

FIG. 4. Stimulation of tPA-mediated plasmin formation by Aβ and synergistic stimulation of cell detachment by plasminogen and Aβ. (A) Plasminogen (200 μ/ml) and tPA (200 pM) were incubated with Aβ (5 μ) or control buffer. Samples were taken from the reaction mixture at the indicated periods of time and plasmin activity was measured by conversion of the chromogenic plasmin subtrate S-2251 at 405 nm. (B) N1E-115 cells were differentiated and received the indicated concentrations of plasmin in the presence or absence of 25 μM Aβ. After 48 hours the dead cells were washed away and the remaining adherent cells were stained with methylene blue. Plasmin cannot prevent Aβ-induced cell detachment. (C) N1E-115 cells were differentiated and received the indicated concentrations of plasminogen in the presence or absence of 10 μM Aβ. After 24 hours cell detachment was then assessed. Aβ or plasminogen alone do not affect cell adhesion, but cause massive cell detachment when added together. (D) Immunoblot analysis of plasmin formation and laminin degradation. Differentiated N1E-115 cells were treated with or without Aβ (10 μM) in the absence or presence of added plasminogen. Addition of Aβ results in the formation of plasmin (bottom panel) and in degradation of laminin (top panel).

FIG. 5. Carboxypeptidase B inhibits Aβ stimulated tPA-mediated plasmin formation and cell detachment. (A) Plasminogen (200 μg/ml) and tPA (200 μM ) were incubated with Aβ (5 μM) or control buffer. Samples were taken from the reaction mixture at the indicated periods of time and plasmin activity was measured by conversion of the chromogenic plasmin subtrate S-2251 at 405 nm. The reaction was performed in the absence or the presence of 50 μg ml⁻¹ carboxypeptidase B (CpB) and in the absence or presence of 3.5 μM carboxypeptidase inhibitor (CPI). CpB greatly attenuates A-stimulated plasmin formation. (B) N1E-115 cells were differentiated and treated with Aβ (10 μM), plasminogen (Plg, 20 μg ml⁻¹) and/or CpB (1 μM) as indicated. After 24 hours the cells were photographed. (C) Subsequently the cells were washed once with PBS and the remaining cells were quantified as percentage adhered cells by methylene blue staining. (D) Cells were treated as in (B) and (C) and medium and cell fractions were collected and analysed by western blot using an anti-plasmin(ogen) antibody. Aβ stimulates plasmin formation that is inhibited by CpB.

FIG. 6. Endostatin can form fibrils comprising cross-β structure and stimulates plasminogen activation. (A) TEM shows the formation of endostatin fibrils. (B) X-ray analysis reveals the presence of cross-β structure in precipitated (prec.) endostatin. (C) Plasminogen activation assay demonstrating the stimulating activity of cross-β structure containing endostatin on tPA mediated plasmin formation. Aβ is shown for comparison. (D) Analysis of endostatin induced cell death by methylene blue staining. It is seen that only the precipitated form is capable of efficiently inducing cell death. Direct cell death, but not cell detachment is protected in the presence of sufficient glucose. Buffer prec. indicates control buffer.

FIG. 7. IAPP stimulates tPA mediated plasminogen activation. Both full length (fl-hIAPP) and truncated amyloid core (Δ-hIAPP), but not mouse IAPP (Δ-mIAPP) stimulate tPA-mediated plasminogen activation.

FIG. 8. Glycated albumin: Thioflavin T and tPA binding, TEM images, X-ray fibre diffraction. (A) ELISA showing binding of tPA to albumin-g6p. (B) Competition of tPA binding to albumin-g6p by Congo red as determined using ELISA. (C) Fluorescence measurements of Thioflavin T binding to albumin-g6p, which is two-, four-, or 23 weeks incubated. (D) Inhibition of the fluorescent signal obtained upon incubation of 430 nM of albumin-g6p with 19 μM of Thioflavin T by tPA. (E) Spectrophotometric analysis at 420 nm shows that increasing amounts of tPA results in a decrease of the specific absorbance obtained upon incubation of 500 μM of albumin-g6p with 10 μM of Thioflavin T. (G) Electron micrographs showing amorphous precipitates of four-weeks glycated albumin-g6p, (H) bundles of fibrillar aggregates of 23-weeks incubated albumin-g6p. (I) Two-weeks glycated albumin-g6p. (J) X-ray scattering of albumin-g6p (23 weeks). Scattering intensities are colour coded on a linear scale and decreases in the order white-grey-black. Scattering from amorphous control albumin is subtracted, as wells as scattering from the capillary glass wall and from air. d-spacings and the direction of the fibre axis are given and preferred orientations are indicated with arrows. (K) Radial scans of albumin control and albumin-g6p (23 weeks). (L) Radial scan of albumin-g6p (23 weeks), showing repeats originating from fibrous structure, after subtracting background scattering of amorphous precipitated albumin. d-spacings (in Å) are depicted above the peaks. (M) Tangential scans along the 2θ scattering-angles, corresponding to indicated d-spacings. The scans show that the 4.7 Å repeat, which corresponds to the hydrogen-bond distance within individual β-sheets, and the 6 Å repeat, are oriented perpendicular to the 2.3 Å repeat, that runs parallel to the fibre axis. (N) Schematic drawing of the orientation of the cross-β structures in albumin-g6p (23 weeks) amyloid fibrils.

FIG. 9. Fibril formation of human haemoglobin. (A) Binding of tPA to in vitro glycated Hb-g6p. (B) Electron micrographs showing in vitro glycated Hb, which aggregates in an amorphous and fibrous manner.

FIG. 10. Amyloid properties of albumin-AGE are introduced irrespective of the carbohydrate or carbohydrate derivative used for glycation. (A-I) Congo red fluorescence of air-dried albumin preparations. Fluorescence was measured with albumin incubated with buffer (A) or with buffer and NaCNBH₃ (B), with amyloid core peptide of human IAPP (C), Aβ (D), with albumin incubated with g6p (E), glucose (F), fructose (G), glyceraldehyde (H), and glyoxylic acid (I). (J) Thioflavin T—amyloid fluorescence was measured in solution with the indicated albumin preparations. (K-L) Binding of amyloid-binding serine protease tPA to albumin preparations was assayed using an ELISA set-up. In (K) binding of tPA to albumin-glucose, -fructose, -glyceraldehyde, -glyoxylic acid, and albumin-buffer controls is shown. In (L) binding of tPA to positive controls albumin-g6p, Aβ and IAPP is shown, as well as to albumin incubated with control buffer.

FIG. 11. Analysis of Congo red-and tPA binding to Aβ. (A) Binding of tPA to immobilized Aβ , as measured using an ELISA. (B) Influence of increasing concentrations of Congo red on binding of tPA to Aβ. In the ELISA 10 μg ml⁻¹ of Aβ(1-40) was coated and incubated with 40 nM of tPA and 0-100 μM of Congo red.

FIG. 12. Binding of human FXII to amyloid peptides and proteins, that contain the cross-β structure fold. (A-B) Binding of FXII to prototype amyloid peptides hAβ(1-40) and human fibrin fragment α¹⁴⁷⁻¹⁵⁹ FP13, and albumin-AGE and Hb-AGE, that all contain cross-β structure, was tested in an ELISA. FXII does not bind to negative controls mouse Δ islet amyloid polypeptide (ΔmIAPP), albumin-control and Hb-control, that all three lack the amyloid-specific structure. kD's for hAβ(1-40), FP13, albumin-AGE and Hb-AGE are approximately 2, 11, 8 and 0.5 nM, respectively. (C-D) Coated hAβ(1-40) was incubated with 2.5 nM FXII in binding buffer, in the presence of a concentration series of human recombinant tissue-type plasminogen activator (Actilyse®, full-length tPA), or Reteplase® (K2P-tPA). The f.l. tPA- and K2P-tPA concentration was at maximum 135 times the kD for tPA binding to hAβ(1-40) (50 nM). (E-F) Coated amyloid albumin-AGE was incubated with 15 nM FXII in binding buffer, in the presence of a concentration series of f.l. tPA or K2P-tPA. The tPA concentration was at maximum 150 times the kD for tPA binding to albumin-AGE (1 nM). (G) Binding of FXII to hAβ(1-40) and the prototype amyloid human amylin fragment HAIAPP was tested using dot blot analysis. 10 μg of the peptides, that contain cross-β structure, as wells as the negative control peptide MΔIAPP and phosphate-buffered saline (PBS) were spotted in duplicate. FXII specifically bound to hAβ(1-40), as well as to hΔIAPP.

FIG. 13. Finger domains bind to amyloid (poly)peptides (A) Binding of tPA and K2-P tPA to albumin-g6p. (B) Binding of tPA and K2-P tPA to Aβ(1-40). The tPA antibody used for detection recognizes both tPA and K2-P-tPA with equal affinity (not shown). (C) Binding of tPA-F-GST and tPA to immobilized Aβ(1-40) and albumin-g6p. Control RPTPμ-GST does not bind Aβ or albumin-g6p. (D) Pull-down assay with insoluble Aβ fibrils and tPA domains. Conditioned BHK medium from stably transfected cell-lines expressing tPA F, F-EGF, EGF, F-EGF-K1 and K1 with a C-terminal GST tag, as wells as the tag alone, was used. “Control,” medium before the pull-down, “Aβ ,” washed amyloid Aβ pellet, after the pull-down, “Sup,” medium after extraction with Aβ. Samples were analyzed on Western blot using rabbit anti-GST antibody Z-5. (E-G) ELISA showing binding of tPA F-EGF-GST and fl. recombinant tPA to amyloid Aβ(E), FP13 (F) and IAPP (G). MΔIAPP was coated as non-amyloid negative control (E). Peptides were immobilized on ELISA plates and overlayed with concentration series of tPA and F-EGF-GST. GST was used as a negative control. Binding was detected using rabbit anti-GST antibody Z-5. (H-M) Immunohistochemical analysis of binding of tPA F-EGF-GST to amyloid deposits in human brain inflicted by Aβ. Brain sections were overlayed with tPA F-EGF-GST (H, J) or negative control GST (L). The same sections were incubated with Congo red (I, K, M) to locate amyloid deposits. (N-O) Pull-down assay with insoluble Aβ fibrils and finger domains. Recombinant F domains with a C-terminal GST tag were expressed by stably transfected BHK cells. “Control,” medium before the pull-down, “Aβ ,” washed amyloid Aβ pellet, after the pull-down, “Sup,” medium after extraction with Aβ. Samples were analyzed on Western blot using rabbit anti-GST antibody Z-5.

FIG. 14. The finger module. (A) Schematic representation of the location of the finger domain in tPA, factor XII, HGFa and fibronectin. (B) Alignment of the amino acid sequence of the finger domain of the respective proteins. (C) Representation of the peptide backbone of the tPA finger domain and the fourth and fifth finger domain of FN. Conserved disulfide bonds are shown in ball and stick.

FIG. 15. Antibodies elicited against amyloid peptides cross-react with glycated proteins, and vice versa. (A-C) ELISA with immobilized g6p-glycated albumin-AGE:23 and Hb-AGE, their non-glycated controls (A), Aβ(1-40) (B), and IAPP and mΔIAPP (C). For the Aβ ELISA, polyclonal anti-human vitronectin antibody a-hVn K9234 was used as a negative control. (D) Binding of α-AGE1 to immobilized Aβ(1-40) on an ELISA plate, after pre-incubation of α-AGE1 with IAPP fibrils. (E) Pull-down assay with anti-AGE1 antibody and amyloid fibrils of Aβ(16-22) (lane 1-2), Aβ(1-40) (lane 4-5) and IAPP (lane 6 -7). After pelleting and washing of the fibrils, samples were boiled in non-reducing sample buffer and analysed by SDS-PAGE. s=supernatant after amyloid extraction, p=amyloid pellet after extraction, m=molecular marker. (F-G) In an ELISA set-up, immobilized Aβ(1-40) (F) and IAPP (G) are co-incubated with tPA and 250 or 18 nM α-AGE1, respectively. (H) In an ELISA set-up binding of α-Aβ(1-42) H-43 to immobilized positive control Aβ(1-40), and to IAPP and albumin-AGE:23 is tested. Albumin-control:23 and mALIPP are used as negative controls. (I) Binding of 100 nM α-Aβ(1-42) H-43 to IAPP, immobilized on an ELISA plate, in the presence of a concentration series of tPA. (J-K) ELISA showing binding of a polyclonal antibody in mouse serum elicited against albumin-AGE:23 and Aβ(1-40) (ratio 9:1) (“poab anti-amyloid”) and of a polyclonal antibody elicited against a control protein (“control serum”) to immobilized IAPP (J) and albumin-AGE:23 (K). Serum was diluted in PBS with 0.1% v/v Tween20. (L) ELISA showing binding of mouse poab anti-amyloid serum to amyloid Aβ(1-40), hΔIAPP and fibrin fragment α₁₄₈₋₁₆₀ FP13. Control serum with antibodies raised against an unrelated protein, buffer and immobilized non-amyloid MΔIAPP and fibrin fragment α₁₄₈₋₁₅₇ FP10were used as negative controls. (M) Immunohistochemical analysis of the binding of rabbit anti-AGE2 to a spherical amyloid plaque (arrow) in a section of a human brain inflicted by Aβ. Magnification 400×. (N) Congo red fluorescence of the same section. Magnification 630×.

FIG. 16 Monoclonal anti-crossβ structure antibody 3H7 detects glycated haemoglobin, Aβ, IAPP and FP13 ELISA showing binding of mouse monoclonal anti-cross-β structure antibody 3H7 to (A) glycated haemoglobin vs control unglycated haemoglobin or (B) Aβ, hIAPP, ΔmIAPP and fibrin fragment α₁₄₈₋₁₆₀ FP13.

FIG. 17. Sandwich ELISA for detection of amyloid albumin-AGE or amyloid haeglobin in solution Immobilized recombinant tPA on Exiqon protein Immobilizers was overlayed with albumin-AGE:23 solution or albumin-control:23 solution at the indicated concentrations. Next, bound amyloid structures were detected with anti-Aβ(1-42) H-43 (A).

FIG. 18. Binding of tPA, factor XII, fibronectin and finger domains thereof to compounds with cross-β structure. A-C. Full-length purified tPA (A), factor XII (B) and fibronectin (C) bind to immobilized peptides with cross-β structure conformation, in an ELISA. D-F. In an ELISA, the recombinant Fibronectin type I, or finger domains (F) of tPA (D), factor XII (E) and fibronectin (F) bind specifically to immobilized amyloid-like peptides with cross-β structure conformation. The control free GST tag does not bind. G. In an ELISA, recombinantly expressed fibronectin type I domains 4-5 of fibronectin, N-terminally tagged to growth hormone and a His₆-tag, and C-terminally tagged to a His₆-tag (FnF4,5his), specifically captures Hb-AGE from solution, and not control haemoglobin. H-I. Activation of the contact system and the fibrinolytic system in patients with systemic amyloidosis. H. Plasma samples of 40 apparently healthy controls (19 male, 21 female, average age 49.4 (stand. dev. 6.8 years)) and of 40 patients with systemic amyloidosis (17 male, 23 female, average age 51.8 (stand. dev. 9.9 years)) were tested for plasmin-α2-anti-plasmin (PAP) levels with an ELISA. One patient revealed a PAP level of 88,3 μg ml⁻¹, which is not shown in the graph for clarity. I. In the same plasma samples levels of FXIIa were measured with an ELISA. Sixteen out of 40 patients with systemic amyloidosis have elevated levels of FXIIa.

FIG. 19. Activation of factor XII by kaolin and by peptide aggregates with cross-β structure conformation. A. Like kaolin, amyloid-like peptide aggregates of FP13 and Aβ stimulate the activation of factor XII, as detected by the conversion of Chromozym PK, upon formation of kallikrein from prekallikrein by activated factor XII. Buffer control and non-amyloid controls FP 10 and mIAPP do not activate factor XII. B. Like FP 13 and Aβ, also cross-β structure rich peptides LAM 12 and TTR11 stimulate factor XII activation, to a similar extent as kaolin. C. In the chromogenic factor XII/kallikrein activity assay, the stimulatory activity of 150 μg/ml kaolin is strongly dependent on the presence of 1 mg/ml albumin in the assay buffer. Albumin alone also shows to some extent factor XII/prekallikrein activating properties. D. Contacting plasma, lysozyme and γ-globulins to DXS500k results in activation of tPA and plasminogen, as measured in the chromogenic tPA/Plg activation assay. DXS500k alone also results in some activation. Plasma, lysozyme or γ-globulins controls do not activate tPA and Plg. E. Overnight incubation at room temperature of plasma with kaolin or DXS500k results in increased fluorescence of amyloid dye Thioflavin T, when compared to incubation with buffer. F. Incubation of γ-globulins with kaolin or DXS500k also induces increased ThT fluorescence. G. Only DXS500k induces ThT fluorescence with lysozyme. Kaolin incubation results only in a small increase in ThT fluorescence, when compared to buffer. H-K. In an ELISA set-up tPA binds specifically to plasma proteins (H), γ-globulins (I), lysozyme (J) and factor XII (K) that were pre-incubated overnight with DXS500k, whereas tPA does not bind to buffer-incubated proteins. K2P tPA that lacks the F domain does not bind to surface-contacted proteins. L. In the tPA ELISA glycated haemoglobin (Hb-AGE) with amyloid-like properties was used as a positive control for tPA binding. M. Auto-activation of factor XII is established by incubating purified factor XII with DXS5OOk or with various amyloid-like protein aggregates with cross-β structure conformation, in the presence of chromogenic substrate S-2222.

FIG. 20. Proteins and peptides with amyloid cross-β structure activate blood platelets and can induce platelet aggregation. A-D. Combined freshly isolated platelets from three healthy human donors are incubated with concentration series of proteins and peptides with cross-β structure conformation, and with controls. Activation of the platelets is analyzed on Western blots by measuring phosphorylation of p38MAPK after 1 minute and 5 minutes of peptide/protein incubation. Concentrations used were 6.25, 25 and 100 μg/ml for haemoglobin, and 5, 25 and 125 μg/ml for the other peptides/proteins used. A-B. Incubation of platelets with amyloid haemoglobin-AGE results in platelet activation similar to the positive control native low density lipoprotein after 1 minute (A). After 5 minutes, Hb-AGE shows a prolonged activation whereas p38MAPK is not phosphorylated by nLDL stimulation anymore (B). Incubation with control haemoglobin results in background levels of p38MAPK phosphorylation, similar to buffer. C-D. Amyloid peptides FP13 and Aβ already potently induce p38MAPK phosphorylation after 1 minute incubation (C), whereas amyloid denatured γ-globulins and transthyretin amyloid fragment TTRl I induce p38MAPK phosphorylation only after 5 minutes stimulation (D). E. In a blood platelet aggregometer the influence of amyloid FP13 and denatured γ-globulins is tested and compared to the effect of thrombin on aggregation. Negative controls were HEPES Tyrode buffer and 200 μg/ml native γ-globulins. Both FP13 and denatured y-globulin induce platelet aggregation in a dose dependent manner. F. Storage of fresh platelets for 72 hours at room temperature results in increased Thioflavin T fluorescence.

FIG. 21. Amyloid-like conformations are presented on activated blood platelets and contribute to platelet aggregation. A-B. Analysis of amyloid formation during adhesion of platelets in whole blood to vWF (A) or collagen (B) under flow for 5 minutes. Samples were stained with the amyloid specific dye Congo Red. C-D. FACS analysis of platelets stimulated with (D) or without (C) thrombin (1 min, 37° C.) in the presence of EDTA. The fluorescent amyloid dye Thioflavin T was used to detect amyloid on the surface of platelets. E. Washed platelets were exposed to thrombin activating peptide (TRAβ ) in the presence or absence of ThT (200 μM), Congo Red (200 μM) or tPA (1μM) where indicated. Platelet aggregation was assessed by light scattering. F. Activation of platelets in the presence of TRAβ , indo and AR, with or without tPA. Indo: indomethacin (aspirin-like), AR-C6993MX (clopidogrel-like). TPA further decreases the level of TRAβ-induced platelet activation, that is suppressed by indo and AR.

FIG. 22: Binding of factor XII and tPA to β2-glycoprotein I and binding of anti-β2gpi auto-antibodies to recombinant β2gpi. A. Chromogenic plasmin assay showing the stimulatory activity of recombinant β2gpi on the tPA-mediated conversion of plasminogen to plasmin. The positive control was amyloid fibrin peptide FP13. B. In an ELISA, recombinant β2gpi binds to immobilized tPA, whereas β2gpi purified from plasma does not bind. The k_{D} is 2.3 μg/ml (51 nM). C. In an ELISA, factor XII binds to purified recombinant human β2gpi, and not to β2gpi that is purified from human plasma, when purified factor XII is immobilized onto ELISA plate wells. Recombinant β2gpi binds with a kD of 0.9 zg/ml (20 nM) to immobilized factor XII. D. Western blot incubated with anti-human factor XII antibody. The β2gpi was purified either from fresh human plasma or from plasma that was frozen at −20° C. and subsequently thawed before purification. When comparing lanes 2-3 with 4-5, it is shown that freezing-thawing of plasma results in co-purification of factor XII together with the β2gpi. The molecular mass of factor XII is 80 kDa. E. In an ELISA recombinant β2gpi efficiently inhibits binding of anti-2gpi auto-antibodies to immobilized β2gpi, whereas plasma β2gpi has a minor effect on antibody binding. Anti-2gpi auto-antibodies were purified from plasma of patients with the auto-immune disease Anti-phospholipid syndrome. F. Exposure of 25 jig/ml β2gpi, recombinantly produced (rβ2gpi) or purified from plasma (nβ2gpi), to 100 μM cardiolipin vesicles or to 250 μg/ml dextransulphate 500,000 Da (DXS) induces an increased fluorescence of Thioflavin T, suggestive for an increase in the amount of cross-β structure in solution. Signals are corrected for background fluorescence of cardiolipin, DXS, ThT and buffer. G. Recombinant β2gpi binds to a higher extent to tPA, which is immobilized on the wells of an ELISA plate, than β2gpi purified from human plasma. H. Binding of tPA and K2P tPA to β2gpi immobilized on the wells of an ELISA plate, or to β2gpi bound to immobilized cardiolipin is assessed. B2gpi contacted to cardiolipin binds tPA to a higher extent than β2gpi contacted to the ELISA plate directly. K2P tPA does not bind to β2gpi. TPA does not bind to immobilized cardiolipin. I. Recombinant β2gpi induces platelet activation as assayed by measuring the extent of platelet p38MAPK phosphorylation. In contrast, β2gpi purified from human plasma induced p38MAPK phosphorylation to a lesser extent.

FIG. 23. Amyloid-like properties of oxidized low-density lipoprotein A. In time, an increase of the oxidation of LDL, as measured by specific diene fluorescence at 243 nm, is accompanied by an increase in Thioflavin T fluorescence and a decrease in Congo red fluorescence, indicative for structural changes in the apoB protein part of the LDL. B. Congo red fluorescence of 25 μg/ml oxidized LDL is similar to the Congo red fluorescence of the positive control, 25 g/ml Aβ. Native LDL also shows Congo red fluorescence to some extent. C. In the chromogenic plasmin assay, 24% oxidized LDL shows cofactor activity for the tPA-mediated conversion of plasminogen to plasmin, whereas native LDL has hardly any effect on tPA activity. D. Factor XII in plasma is activated by oxidized LDL and by amyloid peptide FP13 K157G, as determined with a chromogenic factor XII activation assay using chromogenic substrate S-2222.

FIG. 24. A fibrin clot comprises amyloid-like cross-β structure conformation. A. Fibrin clots show fluorescent signals when stained with amyloid specific dyes Congo red, Thioflavin S and Thioflavin T. As a control, images of fibrin clots as seen under direct light and under the FITC- and TRITC excitation wavelengths are shown. B. Fibrin clots stain positive with Congo red (CR), Thioflavin S (ThS) and Thioflavin T (ThT), indicative for the presence of amyloid-like cross-β structure aggregates. C-E. In an aPTT coagulation test coagulation of human plasma is delayed in the presence of amyloid-specific dyes Congo red (C), ThS (D) and ThT (E), whereas buffer controls do not influence coagulation (Na₂SO₄ for CR and NH₄Cl for ThT/ThS, respectively). These observations are indicative for a role of amyloid-like cross-β structure conformation in the formation of a fibrin polymer network. F-H. In PT coagulation tests, similar inhibitory activities of amyloid-specific dyes CR (F), ThS (G), and ThT (H) on fibrin clot formation are observed as in aPTT's.

FIG. 25. Overview of the domain structure of tPA, Factor XII and Fibronectin, all recombinant constucts made thereof and primers used for preparing the recombinant constucts.

### DETAILED DESCRIPTION OF THE INVENTION

The invention discloses (i) the identification of a “cross-β structre pathway,” (ii) the identification of multiligand receptors as being cross-β structure receptors, (iii) the identification of the finger domain as a cross-β binding module and (iv) the identification of finger containing proteins, including tPA, FXII, HGFa and fibronectin as part of the “cross-β structure pathway.”

This invention further provides compounds not previously known to bind cross-β structure.

As disclosed herein the invention provides compounds and methods for the detection and treatment of diseases associated with the excessive formation of cross-β structure. Such diseases include known conformational diseases, including Alzheimer disease and other types of amyloidosis. However, our invention discloses also that other diseases, not yet known to be associated with excessive formation of cross-β structure are also caused by excessive formation of cross-β structure. Such diseases include atherosclerosis, sepsis, diffuse intravascular coagulation, hemolytic uremic syndrome, preeclampsia, rheumatoid arthritis, autoimmune diseases, thrombosis and cancer.

According to the invention the compound is a cross-β structure binding molecule, preferably, a finger domain or a molecule containing one or more finger domains, or is a peptidomimetic analog of one or more finger domains. The compound can also be an antibody or a functional fragment thereof directed to the cross-β structure.

According to the invention the compound may also be a multiligand receptor of fragment thereof. The compound may e.g., be RAGE, CD36, Low density lipoprotein Related Protein (LRP), Scavenger Receptor B-1 (SR-B1), SR-A or a fragment of one of these proteins.

The finger domains, finger containing molecules or antibodies may be human, mouse, rat or from any other species.

According to the invention amino acids of the respective proteins may be replaced by other amino acids which may increase/decrease the affinity, the potency, bioavailability and/or half-life of the peptide. Alterations include conventional replacements (acid-acid, bulky-bulky and the like), introducing D-amino acids, making peptides cyclic, etc.

Furthermore the the invention provides compounds and methods:
1) for detecting the presence of the cross-β structure.
2) for inhibiting the formation of amyloid fibrils.
3) for modulating cross-β structure induced toxicity.
4) for the removal of cross-β structure containing molecules from the circulation.

This invention provides methods for preparing an assay to measure cross-β structure in sample solutions.

This invention provides methods for detecting cross-β structure in tissue samples or other samples obtained from living cells or animals.

This invention provides compounds and methods for preparing a composition for inhibiting cross-β structure fibril formation.

This invention provides compounds and methods for preparing a composition for modulating cross-β structure induced toxicity.

Abbreviations: Aβ , beta-amyloid peptide; AD, Alzheimer disease; AGE, advanced glycation end-products; CpB, carboxypeptidase B; COI (carboxypeptidase inhibitor); ELISA, enzyme-linked immunosorbent assay (ELISA); FN, fibronectin; FXII, factor XII (Hageman factor); HGFa, hepatocyte growth factor activator; IAPP, islet amyloid polypeptide; PCR, polymerase chain reactions (PCR); RAGE, receptor for AGE; tPA, tissue-type plasminogen activator.

The invention provides compounds and methods for the detection and treatment of diseases associated with the excessive formation of cross-β structure.

The cross-β structure can be part of an Aβ fibril or part of another amyloid fibril. The cross-β structure can also be present in denatured proteins.

The invention provides methods to detect the cross-β structure. In one embodiment a cross-β structure binding compound, preferably, a finger domain or a molecule comprising one or more finger modules, is bound or affixed to a solid surface, preferably, a microtiter plate. The solid surfaces useful in this embodiment would be known to one of skill in the art. For example, one embodiment of a solid surface is a bead, a column, a plastic dish, a plastic plate, a microscope slide, a nylon membrane, etc. After blocking, the surface is incubated with a sample. After removal of unbound sample, bound molecules comprising the cross-β structure are subsequently detected using a second cross-β structure binding compound, preferably, an anti-cross-β structure antibody or a molecule containing a finger module. The second cross-β structure compound is bound to a label, preferably, an enzym, such as peroxidase. The detectable label may also be a fluorescent label, a biotin, a digoxigenin, a radioactive atom, a paramagnetic ion, and a chemiluminescent label. It may also be labeled by covalent means such as chemical, enzymatic or other appropriate means with a moiety such as an enzyme or radioisotope. Portions of the above mentioned compounds of the invention may be labeled by association with a detectable marker substance (e.g., radiolabeled with ¹²⁵I or biotinylated) to provide reagents useful in detection and quantification of compound or its receptor bearing cells or its derivatives in solid tissue and fluid samples such as blood, cerebral spinal fluid, urine or other. Such samples may also include serum used for tissue culture or medium used for tissue culture.

In another embodiment the solid surface can be microspheres for for example agglutination tests.

In one embodiment the compound , containing a finger module is, used to stain tissue samples. Preferably, the compound is fused to a protein, peptide, such as glutathion-S-tranferase. Alternatively, the compound is coupled to a label. The detectable label may be a fluorescent label, a biotin, a digoxigenin, a radioactive atom, a paramagnetic ion, and a chemiluminescent label. It may also be labeled by covalent means such as chemical, enzymatic or other appropriate means with a moiety such as an enzyme or radioisotope. Portions of the above mentioned compounds of the invention may be labeled by association with a detectable marker substance (e.g., radiolabeled with ¹²⁵I ^{99m}Tc, ¹³¹I, chelated radiolabels, or biotinylated) to provide reagents useful in detection and quantification of compound or its receptor bearing cells or its derivatives in solid tissue and fluid samples such as blood, cerebral spinal fluid or urine. The compound is incubated with the sample and after washing visualized with antibodies directed against the fused protein or polypeptide, preferably, glutathion-S-transferase.

In an embodiment the above sample is tissue from patients with or expected to suffer from a conformational disease. Alternatively, the tissue is derived from animals or from cells cultured in vitro.

The methods of the invention provide a new diagnostic tool. It was not until the present invention that a universal β-structure epitope was disclosed and that a diagnostic assay could be based on the presence of the cross-β structure. Such use is particular useful for diagnostic identification of conformational diseases or diseases associated with amyloid formation, like Alzheimer or diabetes. It is clear that this diagnostic use is also useful for other diseases which involve cross-β structure formation, like all amyloidosis type diseases, atherosclerosis, diabetes, bleeding, cancer, sepsis and other inflammatory diseases, Multiple Sclerosis, auto-immune diseases, disease associated with loss of memory or Parkinson and other neuronal diseases (epilepsy). For example, one can use a finger domain (of for example tPA) and provide it with a label (radio active, fluorescent etc.). This labeled finger domain can then be used either in vitro or in vivo for the detection of cross-β structure comprising proteins, hence for determining the presence of a plaque involved in a conformational disease. One can for example use an ELISA assay to determine the amount of sepsis in a patient or one can localize a plaque involved in a conformational disease.

In another embodiment this invention provides a method for inhibiting the formation of amyloid fibrils or to modulate cross-β structure induced toxicity. The compound is a cross-β binding module, preferably, a finger domain, a finger domain containing molecule, a peptidomimetic analog, and/or an anti-cross-β structure antibody, and/or a multiligand receptor or a fragment thereof.

According to the invention, the inhibition of fibril formation preferably, has the consequence of decreasing the load of fibrils.

The inhibition of fibril formation or modulating cross-β strcuture toxicity may also have the consequence of modulating cell death. The cell can be any cell, but preferably, is a neuronal cell, an endothelial cell, or a tumor cell. The cell can be a human cell or a cell from any other species.

The cell may typically be present in a subject. The subject to which the compound is administered may be a mammal or preferably, a human.

The subject may be suffering from amyloidoses, from another conformational disease, from prion disease, from chronic renal failure and/or dialysis related amyloidosis, from atheroscleroses, from cardiovascular disease, from autoimmune disease, or the subject may be obese. The subject may also be suffering from inflammation, rheumatoid arthritis, diabetes, retinopathy, sepsis, diffuse intravascular coagulation, hemolytic uremic syndrome, and/or preeclampsia, The diseases which may be treated or prevented with the methods of the present invention include but are not limited to diabetes, Alzheimer disease, senility, renal failure, hyperlipidemic atherosclerosis, neuronal cytotoxicity, Down's syndrome, dementia associated with head trauma, amyotrophic lateral sclerosis, multiple sclerosis, amyloidosis, an autoimmune disease, inflammation, a tumor, cancer, male impotence, wound healing, periodontal disease, neuopathy, retinopathy, nephropathy or neuronal degeneration.

The administration of compounds according to the invention may be constant or for a certain period of time. The compound may be delivered hourly, daily, weekly, monthly (e.g., in a time release form) or as a one time delivery. The delivery may also be continuous, e.g., intravenous delivery.

A carrier may be used. The carrier may be a diluent, an aerosol, an aqeuous solution, a nonaqueous solution or a solid carrier. This invention also provides pharmaceutical compositions including therapeutically effective amounts of polypeptide compositions and compounds, together with suitable diluents, preservatives, solubilizers, emulsifiers, adjuvants and/or carriers. Such compositions may be liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), antioxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts.

The administration of compounds according to the invention may comprise intralesional, intraperitoneal, intramuscular or intravenous injection; infusion; liposome-mediated delivery; topical, intrathecal, gingival pocket, per rectum, intrabronchial, nasal, oral, ocular or otic delivery. In a further embodiment, the administration includes intrabronchial administration, anal, intrathecal administration or transdermal delivery.

According to the invention the compounds may be administered hourly, daily, weekly, monthly or annually. In another embodiment, the effective amount of the compound comprises from about 0.000001 mg/kg body weight to about 100 mg/kg body weight.

The compounds according to the invention may be delivered locally via a capsule which allows sustained release of the agent over a period of time. Controlled or sustained release compositions include formulation in lipophilic depots (e.g., fatty acids, waxes, oils). Also included in the invention are particulate compositions coated with polymers (e.g., poloxamers or poloxamines) and the agent coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

The effective amount of the compounds according to the invention preferably, comprise 1 ng/kg body weight to about 1 gr/kg body weight. The actual effective amount will be based upon the size of the compound and its properties.

The activity of tPA and/or the tPA mediated activation of plasminogen is increased by the binding to fibrin fragments, or other protein fragments that have a lysine or an arginine at the carboxy-terminal end. B-type carboxypeptidases, including but not limited to carboxypeptidase B (CpB) or Thrombin Activatable Fibinolysis Inhibitor (TAFI, also named carboxypeptidase U or carboxypeptidase R), are enzymes that cleave off carboxy-terminal lysine and arginine residues of fibrin fragments that would otherwise bind to tPA and/or plasminogen and stimulate plasmin formation.

Because this invention has made clear that the cross-β structures are harmful when present in certain parts of the body, like for example the brain, and the damage is effected by the combination of cross-β structures with tPA, a method is provided to inhibit cross-β structure-mediated effects comprising providing an effective amount of a protein comprising a finger domain to block the binding sites of the cross-β structure for tPA. The cross-β structure-mediated effects may even be further diminished comprising providing an effective amount of B-type carboxypeptidase activity to inhibit the tPA activity.

The invention provides the use of a compound capable of binding to a cross-β structure for the removal of cross-β structures. The compound is a cross-β binding molecule, preferably, a protein and/or a functional equivalent and/or a functional fragment thereof. More preferably, the compound comprises a finger domain or a finger domain containing molecule or a functional equivalent or a functional fragment thereof. Even more preferably, the finger domain is derived from fibronectin, FXII, HGFa or tPA. It is clear that the invention also comprises antibodies that bind cross-β structures. In another preferred embodiment the protein is an antibody and/or a functional equivalent and/or a functional fragment thereof. With this use the invention provides for example a therapeutic method to remove cross-β structure comprising proteins from for example the circulation, preferably, via extracorporeal dialysis. For example, a patient with sepsis is subjected to such use by dialysis of blood of the patient through means which are provided with for example, preferably, immobilized, finger domains. One could for example couple the finger domains to a solid surface or to the inside of the tubes used for the dialysis. In this way, all cross-β structure comprising proteins will be removed from the blood stream of the patient, thereby relieving the patients of the negative effects caused by the cross-β structure comprising proteins. Besides finger domain comprising compounds, it is also possible to use other cross-β structure binding compounds, like antibodies or soluble multiligand receptors. It is also clear that the use could be applied in haemodialysis of kidney patients.

As used herein “finger” encompasses a sequence that fullfills the criteria outlined in FIG. 14. The sequence encompasses approximately 50 amino acids, containing 4 cysteine residues at distinct spacing. Preferably, the finger domains of tPA, FXII, HGFa or fibronectin are used. Alternatively, the “finger” may be a polypeptide analog or peptidomimetic with similar funtion, e.g., by having 3-dimensional conformation. It is feasible that such analogs have improved properties.

As mentioned before, factor XII contains a finger d6main and is part of the “cross-beta structure pathway.” Factor XII is important since it activates the intrinsic coagulation pathway. We now provide experimental evidence that factor XII is activated by kaolin and by peptide aggregates with cross-β structure conformation. This shows that the intrinsic route of coagulation is part of the cross-β pathway and that cross-β structure compounds activate coagulation via factor XII. Moreover, we also provide experimental evidence disclosing that blood platelets become activated by cross-β structures. In this way the cross-β structure contributes to coagulation via platelet aggregation and thrombosis. Cross-0 structures also contribute to coagulation via extrinsic pathway and thrombosis via induction of the expression of tissue factor (TF) and by stimulating its release. Cross-β structures induce TF after binding to receptors on the surface of endothelial cells, monocytes, macrophages or other cells (Khechia et al, 1997; Bochkov et al, 2002). Alternatively cross-β structures induce coagulation and/or thrombosis by the fact that fibrin, formed during blood coagulation, comprises cross-β structures, which subsequently activate blood coagulation (Kranenburg et al, 2002). Taken together, it is thus concluded that cross-β structures are important inducers of blood coagulation via activation of factor XII, via activation of platelets and/or via induction of TF expression .and secretion. Thus, blood coagulation is part of the “cross beta structure” pathway. Cross-0 structures bind to compounds of the blood coagulation cascade through a cross-β binding domain (also referred to as “a specific binding partner” or “a specific binding part”).

Cross-beta structures in blood are provided by for example glycated proteins (Bouma et al, 2003), oxidated proteins (Horiuchi et al, 2003; Ursini et al, 2002), unfolded or misfolded proteins (Bucciantini et al, 2002) or pathogenic micro-organisms (Chapman et al, 2002).

The invention provides a method for interfering in blood coagulation and/or thrombosis comprising providing to blood a binding molecule that either binds to a cross-β structure or to a specific cross-β structure binding part of a compound which is part of a blood coagulation cascade. The term blood is to be understood as blood in vivo, i.e., in the circulation of a living animal (or non-human animal) or in vitro, i.e as blood outside of a living animal, for example blood present in a tube. Moreover, the term blood also includes a product derived from blood but still capable of coagulating.

A binding molecule is preferably a bi-specific molecule, i.e., a molecule with two different binding specificities. In a preferred embodiment, the bi-specific molecule is capable of binding to cross-beta as well as to another part of the cross-beta structure and/or of a protein comprising a cross-beta structure. In another preferred embodiment, the bi-specific molecule is capable of binding to a specific binding partner as well as to another part of the same compound which is part of a blood-coagulating cascade. In an even more preferred embodiment of the invention, the bi-specific molecule is an antibody or a finctional part and/or derivative thereof Such a bi-specific antibody is produced as a recombinant molecule and is optionally adapted to the host in which the antibody is used.

In yet another embodiment, the binding molecule is mono-specific and binds to cross-beta structures, for example Congo Red, Thioflavin T, Thioflavin S, CD36, RAGE, scavenger receptor A, LRP, scavenger receptor B-1, C1q, serum amyloid P component, a finger domain comprising protein such as tPA, HGFA, fibronectin, Factor XII or a functional part and/or derivative thereof, i.e., a part or derivative capable of binding to cross-beta structures (Bouma et al, 2002; Horiuchi et al, 2003).

In general all compounds of a blood coagulation cascade that are activated through the binding of a cross-beta structure are suitable points of interfering. Preferred embodiments are a platelet or fibrin or Factor XII or a receptor present on an endothelial cell or a receptor present on a monocyte/macrophage or any other cell exposing multi-ligand receptors and comprising Tissue Factor.

For example, blood platelets are activated after binding of a cross-beta structure. Surprisingly, activated platelets expose cross-beta structures on their surface. The cross-beta structures play a role in the coagulation cascade, for example by enhancing activation of other platelets. Enhancement is artificially decreased by administration of a molecule binding to a cross-beta structure, such as tPA, Congo Red or ThioflavinT. This results in activated platelets that do not aggregate. Platelet activation is also accomplished by fibrin components or thrombin or small peptide compounds, for example TRAβ. These components also induce cross-beta structures on activated platelets. Preferably, activation of a platelet is accomplished by binding of a cross-beta structure to a scavenger/multi-ligand receptor, like for example CD36, LRP, apoER2', scavenger receptor A, scavenger receptor B-I.

Factor XII generally binds to exposed collagen at the site of vesicle wall injury. Factor XII is activated by high molecular weight kininogen and kallikrein. When activated Factor XII becomes a serine protease which activates Factor XI. Activated Factor XII also induces Bradykinin which influences the blood flow. Because cross-beta structures activate Factor XII, they initiate the blood coagulation cascade. Interfering in this pathway with for example the binding of a (mono or) bi-specific molecule to a cross beta structure results in decreased blood coagulation.

Another way of interfering in a blood coagulation cascade is by interfering the binding of cross-beta structures to a receptor present on an endothelial cell or a receptor present on a monocyte/macrophage or any other cell exposing multi-ligand receptors and comprising Tissue Factor. Examples of such receptors are CD36, LRP, scavenger receptor A, scavenger receptor B-I, RAGE, FEEL-1, LOX-1, stabilin-1, stabilin-2 (Bouma et al 2003; Horiuchi et al,

Tamura et al, 2003; Jono et al 2002; Chen et al 2003). We now present evidence that at least endothelial cells and macrophages/monocytes comprise a multi-ligand receptor as well as Tissue Factor. These examples disclose that any cell comprising the combination of a multi-ligand receptor as well as Tissue Factor is capable of initiating or enhancing a blood coagulation cascade.

A (mono or) bi-specific molecule, as described above is useful in the preparation of a medicament for the treatment of coagulation disorders, such as, but not limited to thrombosis.

A pharmaceutical comprising a (mono or) bi-specific molecule of the invention is very useful for treating a diverse range of blood coagulation disorders.

In another embodiment, the invention provides a method for initiating or increasing a blood-coagulating cascade by providing cross-beta structures to blood. For example, local blood clotting is induced in the case of blood loss through vascular disruption. An example of a cross-beta comprising protein is fibrin.

In yet another embodiment, the invention provides a bi-specific molecule capable of binding to a specific cross-beta structure binding part of a compound which is part of a blood-coagulating cascade as well as to another part of the compound or a (mono or) bi-specific molecule capable of binding to cross-beta as well as to another part of the same cross-beta structure. Preferably, such a (mono or) bi-specific molecule is an antibody.

The invention furthermore provides a pharmaceutical comprising a (mono or) bi-specific molecule as described above.

### Experimental Part

Reagents

Bovine serum albumin (BSA) fraction V pH 7.0 and D-glucose-6-phosphate di-sodium (g6p), D, L-glyceraldehyde, and chicken egg-white lysozyme were from ICN (Aurora, Ohio, USA). Rabbit anti-recombinant tissue-type plasminogen activator (tPA) 385R and mouse anti-recombinant tPA 374B were purchased from American Diagnostica (Veenendaal, The Netherlands). Anti-laminin (L9393) was from Sigma. Swine anti-rabbit immunoglobulins/HRP (SWARPO) and rabbit anti-mouse immunoglobulins/HRP (RAMPO) were from DAKO Diagnostics B.V. (The Netherlands). Alteplase (recombinant tissue type plasminogen activator, tPA) was obtained from Boehringer-Ingelheim (Germany). Reteplase (Rapilysin), a recombinant mutant tPA containing only kringle2 and the catalytic domain (K2P-tPA) was obtained from Roche, Hertfordshire, UK, and porcine pancreas carboxypeptidase B (CpB) was from Roche, Mannheim, Germany. Carboxypeptidase inhibitor (CPI) was from Calbiochem (La Jolla, Calif., USA). Tween20 was purchased from Merck-Schuchardt (Hohenbrunn, Germany). Congo red was obtained from Aldrich (Milwaukee, Wis., USA). Thioflavin T and lyophilized human haemoglobin (Hb) were from Sigma (St. Louis, Mo., USA). Lyophilized human fibrinogen was from Kordia (Leiden, The Netherlands). Chromogenic plasmin substrate S-2251 was purchased from Chromogenix (Milan, Italy). Oligonucleotides were purchased from Sigma-Genosys (U.K.). Boro glass-capillaries (0.5 mm Ø) were from Mueller (Berlin, Germany).

Synthetic Peptides

Peptide Aβ(1-40), containing amino acids as present in the described human Alzheimer peptide (DAEFRHDSGYEVHHQKLVFFAEDVGSNKGAIIGLMVGGVV), fibrin peptides 85 (or FP13) (KRLEVDIDIKIRS), 86 (or FP12) (KRLEVDIDIKIR) and 87 (or FP10) (KRLEVDIDIK), derived from the sequence of human fibrin(ogen) and the islet amyloid polypeptide (IAPP) peptide or derivatives

(fl-hIAPP: KCNTATCATQRLANFLVHSSNNFGAILSSTNVGSNTY, ΔhIAPP (SNNFGAILSS), AmIAPP (SNNLGPVLPP) were obtained from Pepscan, Inc. (The Netherlands) or from the peptide synthesis facility at the Netherlands Cancer Institute (NCI, Amsterdam, The Netherlands). The peptides were dissolved in phosphate buffered saline (PBS) to a final concentration of 1 mg ml⁻¹ and stored for three weeks at room temperature (RT) to allow formation of fibrils. During this period, the suspension was vortexed twice weekly. After three weeks, the suspension was stored at 4° C. Freeze-dried Aβ(1-40) from the NCI allowed to form cross-β structure in the same way. Cross-β structure formation was followed in time by examination of Congo red binding and green birefringence under polarised light.

Congo Red Binding and Thioflavin T Fluorescence of a Fibrin Clot

For Thioflavin T-fluorescence measurements 1 mg ml⁻¹ of fibrinogen was incubated at 37° C. with 2 U ml⁻¹ of factor IIa in 150 mM NaCl, 20 mM Tris-HCl pH 7.5, 10 mM CaCl₂, 50 μM Thioflavin T. Background fluorescence of a clot was recorded in the absence of Thioflavin T and background Thioflavin T fluorescence was measured in the absence of factor IIa. Fluorescence was measured on a Hitachi F-4500 fluorescence spectrophotometer (Ltd., Tokyo, Japan), using Sarstedt REF67.754 cuvettes. Apparatus settings: excitation at 435 nm (slit 10 nm), emission at 485 nm (slit 10 nm), PMT voltage 950 V, measuring time 10 seconds, delay 0 seconds. For detection of Congo red binding a fibrin clot was formed at room temperature as described above (Thioflavin T was omitted in the buffer). The clot was incubated with Congo red solution and washed according to the manufacturer's recommendations (Sigma Diagnostics, Mo., USA). The clot was analysed under polarised light.

Initial Preparation of Glycated Albumin, Haemoglobin (Hb) and Lysozyme

For preparation of advanced glycation end-product modified bovine serum albumin (albumin-g6p), 100 mg ml⁻¹ of albumin was incubated with PBS containing 1 M of g6p and 0.05% m/v NaN₃, at 37° C. in the dark. One albumin solution was glycated for two weeks, a different batch of albumin was glycated for four weeks. Glycation was prolonged up to 23 weeks with part of the latter batch. Human Hb at 5 mg ml⁻¹ was incubated for 10 weeks at 37° C. with PBS containing 1 M of g6p and .05% m/v of NaN₃. In Addition, a Hb solution of 50 mg ml⁻¹ was incubated for eight weeks with the same buffer. For preparation of glyceraldehyde-modified albumin (albumin-glyceraldehyde) and chicken egg-white lysozyme (lysozyme-glyceraldehyde), filter-sterilized protein solutions of 15 mg ml⁻¹ were incubated for two weeks with PBS containing 10 mM of glyceraldehyde. In controls, g6p or glyceraldehyde was omitted in the solutions. After incubations, albumin and lysozyme solutions were extensively dialysed against distilled water and, subsequently, stored at −20° C. Protein concentrations were determined with Advanced protein-assay reagent ADV01 (Cytoskeleton, Denver, Colo., USA). Glycation was confirmed by measuring intrinsic fluorescent signals from advanced glycation end-products; excitation wavelength 380 nm, emission wavelength 435 nm.

Further Experiment Involving Glycation

For preparation of albumin-AGE, 100 mg ml⁻¹ bovine serum albumin (fraction V, catalogue # A-7906, initial fractionation by heat shock, purity ≧98% (electrophoresis), remainder mostly globulins, Sigma-Aldrich, St. Louis, Mo., USA) was incubated at 37° C. in the dark, with phosphate-buffered saline (PBS, 140 mM sodium chloride, 2.7 mM potassium chloride, 10 mM disodium hydrogen phosphate, 1.8 mM potassium di-hydrogen phosphate, pH 7.3) 1M D-glucose-6-phosphate disodium salt hydrate (anhydrous) (ICN, Aurora, Ohio, USA) and 0.05% (m/v) NaN₃. Bovine albumin has 83 potential glycation sites (59 lysine and 23 arginine residues, N-terminus). Albumin was glycated for two weeks (albumin-AGE:2), four weeks (albumin-AGE:4) or 23 weeks (albumin-AGE:23). In controls, g6p was omitted. After incubation, solutions were extensively dialysed against distilled water and, subsequently, stored at 4° C. Protein concentrations were determined with advanced protein-assay reagent ADV01 (Cytoskeleton, CO, USA). Alternatively, albumin was incubated for 86 weeks with 1 M g6p, 250 mM DL-glyceraldehyde (ICN, Aurora, Ohio, USA)/100 mM NaCNBH₃, 1 M β-D-(-)-fructose (ICN, Aurora, Ohio, USA), 1 M D(+)-glucose (BDH, Poole, England), 500 mM glyoxylic acid monohydrate (ICN, Aurora, Ohio, USA)/100 mM NaCNBH₃, and corresponding PBS and PBS/NaCNBH₃ buffer controls. Glycation was confirmed (i.) by observation of intense brown staining, (ii.) by the presence of multimers on SDS-polyacrylamide gels, (iii) by assaying binding of AGE-specific antibodies moab anti-albumin-g6p 4B5⁴⁶ and poab anti-fibronectin-g6p (Ph. De Groot/I. Bobbink, UMC Utrecht; unpublished data), and (iv.) by measuring intrinsic fluorescent signals from AGE (excitation wavelength 380 nr, emission wavelength 445 nm). Autofluorescent signals of albumin-controls were less than 4% of the signals measured for albumin-AGE and were used for background corrections.

Isolation of Hb from Human Erythrocytes

Human Hb was isolated from erythrocytes in EDTA-anticoagulated blood of 3 healthy individuals and of 16 diabetic patients. 100 μl of whole blood was diluted in 5 ml of physiological salt (154 mM NaCl), cells were gently spun down, and resuspended in 5 ml of physiological salt. After a 16-h incubation at room temp., cells were again spun down. Pelleted cells were lysed by adding 2 ml of 0.1 M of boric acid, pH 6.5 and subsequently, cell debris was spun down. Supernatant was collected and stored at −20° C.

Determination of GlycoHb Concentrations

Concentrations of glycated Hb, also named glycohaemoglobin, or named Hb_{Alc}, in EDTA-blood of human healthy donors or diabetic patients, were determined using a turbidimetric inhibition immunoassay with haemolysed whole blood, according to the manufacturer's recommendations (Roche Diagnostics, Mannheim, Germany). Standard deviations are 2.3% of the measured Hb_{Alc} concentrations.

Binding of Congo Red to Glycated Albumin

Binding of Congo red to albumin-AGE glycated for 86 weeks with carbohydrates glucose, fructose and glucose-6-phosphate, or with carbohydrate derivatives glyceraldehyde and glyoxylic acid, was tested using air-dried samples. For this purpose, 5 μg albumin was applied to a glass cover slip and air-dried. Samples were incubated with Congo red and subsequently washed according to the manufacturer's recommendations (Sigma Diagnostics, St Louis, M0., USA). Pictures were taken on a Leica DMIRBE fluorescence microscope (Rijswijk, The Netherlands) using 596 nm and 620 nm excitation- and emission wavelengths, respectively.

Endostatin Preparations

Endostatin was purified from Escherichia coli essentially as described.⁴⁷ In short, B121.DE3 bacteria expressing endostatin were lysed in a buffer containing 8 M urea, 10 mM Tris (pH 8.0), 10 mM imidazole and 10 mM β-mercapto-ethanol. Following purification over Ni2+-agarose, the protein sample was extensively dialysed against H₂O. During dialysis endostatin precipitates as a fine white solid. Aliquots of this material were either stored at −80° C. for later use, or were freeze-dried prior to storage. Soluble endostatin produced in the yeast strain Pichia pastoris was kindly provided by Dr. Kim Lee Sim (EntreMed, Inc.,Rockville, Mass.). Aggregated endostatin was prepared from soluble endostatin as follows. Soluble yeast endostatin was dialysed overnight in 8 M urea and subsequently three times against H₂O. Like bacterial endostatin, yeast endostatin precipitates as a fine white solid.

Congo Red Staining

Freeze-dried bacterial endostatin was resuspended in, either 0.1% formic acid (FA), or in dimethyl-sulfoxide and taken up in a glass capillary. The solvent was allowed to evaporate and the resulting endostatin material was stained with Congo red according to the manufacturer's protocol (Sigma Diagnostics, St. Louis, Mo., USA).

Circular Dichroism Measurements

UV circular dichroism (CD) spectra of peptide and protein solutions (100 μg ml⁻¹ in H₂O) were measured on a JASCO J-810 CD spectropolarimeter (Tokyo, Japan). Averaged absorption spectra of 5 or 10 single measurements from 190-240 nm or from 190-250 nm, for fibrin peptides 85, 86, 87 or for albumin, glycated albumin and human Aβ(16-22), respectively, are recorded. The CD spectrum of Aβ(16-22) was measured as a positive control. Aβ(16-22) readily adopts amyloid fibril conformation with cross-β structure, when incubated in H₂O.⁴⁵ For albumin and Aβ(16-22) relative percentage of the secondary structure elements present was estimated using k2d software.⁴⁸

X-ray Fibre Diffraction

Aggregated endostatin was solubilized in 0.1% FA, lyophilized fibrin peptides were dissolved in H₂O and glycated albumin was extensively dialysed against water. Samples were taken up in a glass capillary. The solvent was then allowed to evaporate over a period of several days. Capillaries containing the dried samples were placed on a Nonius kappaCCD diffractometer (Bruker-Nonius, Delft, The Netherlands). Scattering was measured using sealed tube MoKa radiation with a graphite monochromator on the CCD area detector during 16 hours. Scattering from air and the glass capillary wall were subtracted using in-house software (VIEW/EVAL, Dept. of Crystal- and Structural Chemistry, Utrecht University, The Netherlands).

Transmission Electron Microscopy

Endostatin-, haemoglobin- and albumin samples were applied to 400 mesh specimen grids covered with carbon-coated collodion films. After 5 minutes, the drops were removed with filter paper and the preparations were stained with 1% methylcellulose and 1% uranyl acetate. After washing in H₂O, the samples were dehydrated in a graded series of EtOH and hexanethyldisilazane. Transmission electron microscopy (TEM) images were recorded at 60 kV on a JEM-1200EX electron microscope (JEOL, Japan).

Enzyme-linked Immunosorbent Assay: Binding of tPA to Glycated Albumin, Hb and Aβ(1-40)

Binding of tPA to albumin-g6p (four-weeks and 23-weeks incubations), albumin-glyceraldehyde, control albumin, human Hb-g6p (ten-weeks incubation), Hb control, or to Aβ(1-40) was tested using an enzyme-linked immunosorbent assay (ELISA) set-up. Albumin-g6p and control albumin (2.5 μg ml⁻¹ in coat buffer, 50 mM Na₂CO₃/NaHCO₃ pH 9.6, 0.02% m/v NaN₃, 50 μl/well) were immobilized for 1 hour at room temperature in 96-well protein Immobilizer plates (Exiqon, Vedbaek, Denmark). Aβ(1-40) (10 tug ml⁻¹ in coat buffer) was immobilized for 75 minutes at room temperature in a 96-well peptide Immobilizer plate (Exiqon, Vedbaek, Denmark). Control wells were incubated with coat buffer, only. After a wash step with 200 μl of PBS/0.1% v/v Tween20, plates were blocked with 300 μl of PBS/1% v/v Tween20, for 2 hours at room temperature, while shaking. All subsequent incubations were performed in PBS/0.1% v/v Tween20 for 1 hour at room temperature while shaking, with volumes of 50 μl per well. After each incubation wells were washed five times with 200 μl of PBS/0.1% v/v Tween20. Increasing amounts of f.l. tPA or K2-P tPA was added in triplicate to coated wells and to control wells. Antibody 385R and, subsequently, SWARPO, or antibody 374B and, subsequently, RAMPO were added to the wells at a concentration of 1 μg ml⁻¹. Bound peroxidase-labeled antibody was visualised using 100 μl of a solution containing 8 mg of ortho-phenylene-diamine and 0.0175% v/v of H₂O₂ in 20 ml of 50 mM citric acid/100 mM Na₂HPO₄ pH 5.0. Staining was stopped upon adding 50 μl of a 2-M H₂SO₄ solution. Absorbance was read at 490 nm on a Vₘₐₓ. kinetic microplate reader (Molecular Devices, Sunnyvale, Calif., USA).

Competition experiments were performed with 20 or 40 nM of tPA, with respectively albumin-g6p or Aβ(1-40) and with increasing amounts of Congo red in PBS/0.08% v/v Tween20/2% v/v EtOH.

ELISA: Binding of tPA to Albumin-AGE

Binding of the cross-β structure-marker tPA to albumin-AGE was tested using an ELISA setup. We showed that tPA binds to prototype amyloid peptides human Aβ(1-40) and human IAPP⁴⁹ (this application). Therefore, we used tPA binding to these two peptides as positive control. The 86-weeks glycated samples and controls were coated to Greiner microlon plates (catalogue # 655092, Greiner, Frickenhausen, Germany). Wells were blocked with Superblock (Pierce, Rockford, Ill., USA). All subsequent incubations were performed in PBS/0.1% (v/v) Tween20 for 1 hour at room temperature while shaking, with volumes of 50 μl per well. After incubation, wells were washed five times with 300 μl PBS/0.1% (v/v) Tween20. Increasing concentrations of tPA were added in triplicate to coated wells as well as to control wells. During tPA incubations of 86-weeks incubated samples, at least a 123,000 times molar excess of ε-amino caproic acid (εACA, 10 mM) was added to the solutions. MACA is a lysine analogue and is used to avoid potential binding of tPA to albumin via its kringle2 domain.⁵⁰ Monoclonal antibody 374b (American Diagnostica, Instrumentation laboratory, Breda, The Netherlands) and, subsequently, RAMPO (Dako diagnostics, Glostrup, Denmark) was added to the wells at a concentration of 0.3 μl ml⁻¹. Bound peroxidase-labeled antibody was visualised using 100 μl of a solution containing 8 mg ortho-phenylene-diamine in 20 ml 50 mM citric acid/100 mM Na₂HPO₄ pH 5.0 with 0.0175% (v/v) H₂O₂. Staining was stopped upon adding 50 μl of a 2 M H₂SO₄ solution. Absorbance was read at 490 nm on a Vman kinetic microplate reader (Molecular Devices, Calif., USA). Background signals from non-coated control wells were substracted from corresponding coated wells.

Initially, Thioflavin T fluorescence of glycated albumin and lysozyme, and tPA

For fluorescence measurements, 500 nM of albumin-g6p, albumin-glyceraldehyde, control albumin, lysozyme-glyceraldehyde, or control lysozyme were incubated with increasing amounts of Thioflavin T, in 50 mM of glycine-NaOH, pH 9. For blank readings, an identical Thioflavin T dilution range was prepared without protein, or Thioflavin T was omitted in the protein solutions. Samples were prepared in triplicate.

Thioflavin T Fluorescence

In further experiments fluorescence measurements, albumin-g6p:2, albumin-g6p:4, albumin-g6p:23 and controls in 50 mM glycine-NaOH, pH 9 were incubated with increasing amounts of ThT (Sigma-Aldrich Chemie, Steinheim, Germany), a marker for amyloid cross-β structure.⁵¹ Albumin-AGE:4 concentration was 175 nM, other protein concentrations were 500 nM. For fluorescence measurements with 86-weeks glycated samples, 140 nM of protein was incubated with a fixed concentration of 20 μThT. Fluorescence was measured in triplicate on a Hitachi F-4500 fluorescence spectrophotometer (Ltd., Tokyo, Japan), after 1 hour incubation at room temperature. Excitation- and emission wavelengths were 435 nm (slit 10 nm) and 485 nm (slit 10 nm), respectively. Background signals from buffer and protein solution without ThT were substracted from corresponding measurements with protein solution incubated with ThT.

Fluorescence: Competitive Binding of Thioflavin T and tPA to Albumin-g6p

A solution of 430 nM albumin-g6p and 19 μM of Thioflavin T was incubated with increasing amounts of tPA, for 1 hour at room temperature. For blank readings, albumin-g6p was omitted. Samples were prepared in four-fold in 50 mM glycine-NaOH pH 9. Emission measurements were performed as described above.

Absorbance: Competitive Binding of Thioflavin T and tPA to Albumin-g6p

Albumin-g6p (500 nM) and Thioflavin T (10 μM) were incubated with increasing amounts of tPA, in 50 mM glycine-NaOH pH 9, for 1 hour at room temperature. Absorbance measurements were performed at the albumin-g6p Thioflavin T absorbance maximum at 420 nm. Samples were prepared in four-fold. For blank readings, albumin-g6p was omitted in the solutions. Absorbance was read in a quartz cuvette on a Pharmacia Biotech Ultrospec 3000 UV/visible spectrophotometer (Cambridge, England).

Plasminogen Activation Assay.

Plasminogen (200 μl ml⁻¹) was incubated with tPA (200 μM) in the presence or the absence of a cofactor (5 μM of either endostatin, Aβ(1-40) or one the fibrin-derived peptides 85, 86 and 87). At the indicated time intervals samples were taken and the reaction was stopped in a buffer containing 5 mM EDTA and 150 mM εACA. After collection of the samples a chromogenic plasmin substrate S-2251 was added and plasmin activity was determined kinetically in a spectrophotometer at 37° C.

N1E-115 Cell Culture and Differentiation

N1E-115 mouse neuroblastoma cells were routinely cultured in DMEM containing 5% FCS, supplemented with antibiotics. Cells were differentiated into post-mitotic neurons.⁵² The cells were exposed to Aβ(50 μg ml⁻¹) for 24 hours in the presence or absence of 20 μg ml⁻¹ lasminogen in the presence or absence of 50 μg ml⁻¹CpB. Cells were photographed, counted and lysed by the addition of 4× sample buffer (250 mM Tris pH 6.8, 8% SDS, 10% glycerol, 100 mM DTT, 0.01% w/v bromophenol blue) to the medium. The lysate, containing both adherent and floating (presumably dying and/or dead) cells as well as the culture medium were analysed for the presence of plasminogen and plasmin as well as for laminin by Western blot analysis using specific antibodies against plasminogen (MoAb 3642, American Diagnostics), laminin (PoAb L9393, Sigma).

Binding of Human Factor XII to Amyloid Peptides and Proteins, that Contain the cross-β Structure Fold

We tested the binding of human FXII (Calbiochem, La Jolla, Calif., USA, catalogue #233490) to amyloid (poly)peptides. Prototype amyloid peptides human amyloid-β(1-40) (hA(1-40)) and human fibrin fragment α₁₄₇₋₁₅₉ FP13, and glucose-6-phosphate glycated bovine albumin (albumin-advanced glycation endproduct (AGE)) and glucose-6-phosphate glycated human haemoglobin (Hb-AGE), that all contain cross-β structure, as well as negative controls mouse A islet amyloid polypeptide (ΔmIAPP), albumin-control and Hb-control, that all three lack the amyloid-specific structure, were coated to ELISA plates and overlayed with a concentration series of human factor XII. Binding of FXII was detected using a rabbit polyclonal anti-FXII antibody (Calbiochem, La Jolla, Calif., USA, catalogue #233504) and peroxidase-labeled swine anti-rabbit IgG. Wells were coated in triplicate. The FXII binding buffer consisted of 10 mM HEPES pH 7.3, 137 mM NaCl, 11 mM D-glucose, 4 mM KCl, 1 mg ml⁻¹ albumin, 50 μM ZnCl₂, 0.02% (m/v) NaN₃ and 10 mM ε-amino caproic acid (εACA). Lysine analogue εACA was added to avoid putative binding of FXII to cross-β structure via the FXII kringle domain. In addition, binding of FXII to hAβ(1-40) and the prototype amyloid human amylin fragment hΔIAPP was tested using dot blot analysis. 10 μg of the peptides, that contain cross-β structure, as wells as the negative control peptide mΔIAPP and phosphate-buffered saline (PBS) were spotted in duplicate onto methanol-activated nitrocellulose. Spots were subsequently incubated with 2 nM FXII in FXII buffer or with FXII buffer alone, anti-FXII antibody, and SWARPO. Binding of FXII was visualized by chemiluminescence upon incubation with enhanced luminol reagent (PerkinElmer Life Sciences, Boston, Mass., USA). To test whether FXII and tPA, which is known for its capacity to bind to polypeptides that contain the cross-β structure fold,⁴⁹ bind to overlapping binding sites on amyloid (poly)peptides, we performed competitive ELISA's. Coated hAβ(1-40) or amyloid albumin-AGE were incubated with 2.5 nM or 15 nM FXII in binding buffer, in the presence of a concentration series of human recombinant tissue-type plasminogen activator (Actilyse®, full-length tPA), or Reteplase® (K2P-tPA). Reteplase is a truncated form of tPA, that consists of the second kringle domain and the protease domain. The f.l. tPA- and K2P-tPA concentration was at maximum 135 times the kD for tPA binding to hAβ(l-40) (50 nM) or 150 times the k_{D} for tPA binding to albumin-AGE (1 nM).

Cloning Procedure

Cloning of the amino-terminal finger domain (F) of human tPA, residues Ser1-Ser50, preceded by the pro-peptide (residues Met-35-Arg-1) and a BglII restriction site, was performed by using PCR and standard recombinant DNA techniques. In brief, the propeptide-finger region was amplified by PCR using 1 ng of plasmin Zpl7,⁵³ containing the cDNA encoding tPA as a template. Oligonucleotides used were 5′-AAAAGTCGACAGCCGCCACCATGGATGCAATGAAGAGA (1) and 3′-AAAAGCGGCCGCCCACTTTTGACAGGCACTGAG (2) comprising a SalI- or a NotI restriction-site, respectively (underlined). The PCR product was cloned in a SalI/NotI-digested expression vector, pMT2-GST.⁵⁴ As a results a construct is generated that contains a SalI restriction site, the coding sequence for the finger domain of tPA, a NotI and a KpnI restriction site, a thrombin cleavage-site (TCS), a glutathion-S-transferase (GST) tag and an EcoRI restriction site. The appropriate sequence of the construct was confirmed by sequence analysis. In a similar way a construct consisting of the tPA F-EGF domains was prepared. Next, the constructs were ligated SalI-EcoRI in pGEM3Zf(−) (Promega, Madison, Wis., USA). The HindIII-SalI-tPA propeptide-BglII-F-NotI-KpnI-TCS-GST-EcoRI construct was used as a cloning cassette for preparation of constructs containing tPA KI, F-EGF-K1, EGF, as wells as human hepatocyte growth factor activator F and F-EGF, human factor XII F and F-EGF, and human fibronectin F4, F5, F4-5 and F10-12. Subsequently, constructs were ligated HindIII-EcoRI in the pcDNA3 expression vector (Invitrogen, Breda, The Netherlands). In addition, the GST tag alone was cloned into pcDNA3, preceded by the tPA propeptide. Primers used for constructs were:

tPA F-EGF
3′-AAAAGCGGCCGCGTGGCCCTGGTATCTATTTC (3) and (1)
tPA EGF
5′-AAAAGAGATCTGTGCCTGTCAAAAGTTGC (4) and (2)
tPA K1
5′-AAAAGAGATCTGATACCAGGGCCACGTGCTAC (5)
3′-AAAAGCGGCCGCCCGTCACTGTTTCCCTCAGAGCA (6)
tPA F-EGF-K1
(1) and (6)
GST tag
(1) and AAAAGCGGCCGCCTGGCTCCTCTTCTGAATC (7)
Fibronectin F4
5′-TGCAAGATCTATAGCTGAGAAGTGTTTTGAT (8)
3′-GATGCGGCCGCCCTGTATTCCTAGAAGTGCAAGTG (9)
Fibronectin F5
5′-TGCAAGATCTACTTCTAGAAATAGATGCAAC (10)
3′-TGATGCGGCCGCCCCACAGAGGTGTGCCTCTC (11)
Fibronectin F4-5
(8) and (11)
Fibronectin F10-12
5′-AAAAAAGATCTAACCAACCTACGGATGACTC (12)
3′-AAAAAAGGTACCGACTGGGTTCACCCCCAGGT (13)
factor XII F
5′-GAAACAAGATCTCAGAAAGAGAAGTGCTTTGA (14)
3′-ACGGGCGGCCGCCCGGCCTGGCTGGCCAGCCGCT (15)
factor XII F-EGF
5′-AAAAAAGATCTCAGAAAGAGAAGTGCTTTGA (16)
3′-AAAAAGGTACCGGCTTGCCTTGGTGTCCACG (17)
HGFaF
5′-GCAAGAAGATCTGGCACAGAGAAATGCTTTGA (18)
3′-AAGGGCGGCCGCCCAGCTGTATGTCGGGTGCCTT (19)
HGFa F-EGF
5′-AAAAAAGATCTGGCACAGAGAATGCTTTGA (20)
3′-AAAAAGGTACCGCTCATCAGGCTCGATGTTG (21)

Transient Expression of tPA-F-GST in 293T Cells

Initially 293T cells were grown in RPMI1640 medium (Invitrogen, Scotland, U.K.) supplemented with 5% v/v fetal calf-serum, penicillin, streptomycin and guanidine, to 15% confluency. Cells were transiently transfected using Fugene-6, according to the manufacturer's recommendations (Roche, Ind., USA). pMT2-tPA-F-GST containing the tPA fragment, or a control plasmid, pMT2-RPTPp-GST, containing the extracellulair domain of receptor-like protein tyrosine phosphatase μ (RPTPμ)⁵⁴ were transfected, and medium was harvested after 48 hours transfection. Expression of tPA-F-GST and RPTPμ-GST in 293T medium was verified by immunoblotting. Collected samples were run out on SDS-PAA gels after the addition of 2× sample buffer. Gels were blotted on nitrocellulose membranes. Membranes were blocked in 1% milk (Nutricia) and incubated with primary monoclonal anti-GST antibody 2F3,⁵⁴ and secondary HRP-conjugated rabbit anti-mouse IgG (RAMPO). The blots were developed using Western Lightning Chemiluminescence Reagent Plus (PerkinElmer Life Sciences, Mass., USA).

Stable Expression of Finger Constructs in BHK Cells

Baby hamster kidney cells were seeded in DMEM/NUT mix F-12(HAM) medium (Invitrogen, U.K.) supplemented with 5% v/v fetal calf-serum (FCS), penicillin, streptomycin and guanidine, to 1% confluency. Cells were stably transfected by using the Ca₃(PO₄)₂-DNA precipitation method. After 24 hours, medium was supplemented with 1 mg ml⁻¹ geneticin G-418 sulphate (Gibco, U.K.). Medium with G-418 was refreshed several times during 10 days, to remove dead cells. After this period of time, stable single colonies were transferred to new culture flasks and cells were grown to confluency. Expression of constructs was then verified by immunoblotting. Collected samples were run out on SDS-PAA gels after the addition of 2× sample buffer. Gels were blotted on nitrocellulose membranes. Membranes were blocked in 5% milk (Nutricia) with 1.5% m/v BSA and incubated with primary monoclonal anti-GST antibody (Santa Cruz Biotechnology, Santa Cruz, Calif., USA, catalogue # Z-5), and secondary HRP-conjugated rabbit anti-mouse IgG (RAMPO). The blots were developed using Western Lightning Chemiluminescence Reagent Plus (PerkinElmer Life Sciences, Mass., USA). Stable clones were from now on grown in the presence of 250 μg ml⁻¹ G-418. For pull-down experiments, conditioned medium with 5% FCS of stable clones that produce constructs of interest was used. For purification purposes, cells of a stable clone of tPA F-EGF-GST were transferred to triple-layered culture flasks and grown in medium with 0.5% v/v Ultroser G (ITK Diagnostics, Uithoorn, The Netherlands). Medium was refreshed every three to four days. TPA F-EGF-GST was isolated from the medium on a Glutathione Sepharose 4B (Amersham Biosciences, Uppsala, Sweden) column and eluted with 100 mM reduced glutathione (Roche Diagnostics, Mannheim, Germany). Purity of the construct was checked with SDS-PAGE followed by Coomassie staining or Western blotting. From these analyses it is clear that some GST is present in the preparation. Purified tPA F-EGF-GST was dialyzed against PBS and stored at −20° C.

Purification of GST-tagged tPA-F-GST and RPTPμ-GST

Medium was concentrated twenty-fold using Nanosep 10K Ω centrifugal devices (Pall Gelman Laboratory, Miss., USA) and incubated with glutathione coupled to Sepharose 4B, according to the manufacturer's recommendations (Pharmacia Biotech, Uppsala, Sweden). Bound constructs were washed with PBS and eluted with 10 mM of glutathione in 50 mM Tris-HCl pH 8.0. Constructs were stored at −20° C., before use.

Amyloid Pull-down

Conditioned medium of BHK cells expressing GST-tagged tPA F, F-EGF, EGF, K1, F-EGF-K1, FXII F, HGFa F, Fn F4, Fn F5, Fn F4-5 and GST was used for amyloid binding assays. At first, constructs were adjusted to approximately equal concentration using Western blots. Qualitative binding of the recombinant fragments are evaluated using a “pull-down” assay. To this end, the recombinantly made fragments, are incubated with either Aβ or IAPP fibrils. Since these peptides form insoluble fibers, unbound proteins can be easily removed from the fibers following centrifugation. The pellets, containing the bound fragments are subsequently washed several times. Bound fragments are solubilized in SDS-sample buffer and analyzed by PAGE, as well as unbound proteins in the supernatant fraction and starting material. The gels are analyzed using immunoblotting analysis with the anti-GST antibody Z-5.

Amyloid ELISA with tPA F-EGF-GST

In order to define the affinity of the purified tPA F-EGF-GST recombinant protein we performed ELISA's with immobilized amyloid (poly)peptides and non-amyloid control ΔmIAPP. Twenty-five μg ml⁻¹ of Aβ , FP13, IAPP or ΔmIAPP was immobilized on Exiqon peptide immoblizer plates. A concentration series of tPA F-EGF-GST in the presence of excess εACA, was added to the wells and binding was assayed using anti-GST antibody Z-5. As a control GST (Sigma-Aldrich, St. Louis, Mo., USA, catalogue # G-5663) was used at the same concentrations.

Immunohistochemistry: Binding of tPA F-EGF to Human AD Brain

Paraffin brain sections of a human inflicted with AD was a kind gift of Prof. Slootweg (Dept. of Pathology, UMC Utrecht). Sections were deparaffinized in a series of xylene-ethanol. Endogenous peroxidases were blocked with methanol/1.5% H₂O₂ for 15 minutes. After rinsing in H₂O, sections were incubated with undiluted formic acid for 10 minutes, followed by incubation in PBS for 5 minutes. Sections were blocked in 10% HPS in PBS for 15 minutes. Sections were exposed for 2 hours with 7 nM of tPA F-EGF-GST or GST in PBS/0.3% BSA. After three wash steps with PBS, sections were overlayed with 200 ng ml⁻¹ anti-GST antibody Z-5, for 1 hour. After washing, ready-to-use goat anti-rabbit Powervision (Immunologic, Duiven, The Netherlands, catalogue # DPVR-55AP) was applied and incubated for 1 hour. After washing, sections were stained for 10 minutes with 3,3′-diamino benzidine (Sigma-Aldrich, St Louis, Mo., USA, catalogue # D-5905), followed by haematoxylin staining for 10 seconds. After washing with H2O, sections were incubated with Congo red according to the manufacturer's recommendations (Sigma Diagnostics, St Louis, Mo., USA). Sections were cleared in xylene and mounted with D.P.X. Mounting Medium (Nustain, Nottingham, U.K.). Analysis of sections was performed on a Leica DMIRBE fluorescence microscope (Rijswijk, The Netherlands). Fluorescence of Congo red was analysed using an excitation wavelength of 596 nm and an emission wavelength of 620 nm.

ELISA: Binding of tPA-F-GST and RPTPl-GST to Human Ab(1-40) and Glycated Albumin

Binding of tPA-F-GST and RPTPl-GST to fibrous amyloids human Aβ (1-40) and albumin-g6p was assayed with an ELISA. In brief, human Aβ (1-40), albumin-g6p, or buffer only, were coated on a peptide I Immobilizer, or a protein I Immobilizer, respectively. Wells were incubated with the purified GST-tagged constructs or control medium, and binding was detected using primary anti-GST monoclonal antibody 2F3 and RAMPO. The wells were also incubated with 500 nM of tPA in the presence of 10 mM of eACA. Binding of tPA is then independent of the lysyl binding-site located at the kringle2 domain. Binding of tPA was measured using primary antibody 374B and RAMPO. Experiments were performed in triplicate and blank readings of non-coated wells were substracted.

Anti-AGE Antibodies

Antibodies against glucose-6-phosphate glycated bovine serum albumin were elicited in rabbits using standard immunization schemes. Anti-AGE1 was obtained after immunization with two-weeks glycated albumin-AGE (Prof. Dr. Ph. G. de Groot/Dr. I. Bobbink; unpublished data). The antibody was purified from serum using a Protein G column. Anti-AGE2 was developed by Davids Biotechnologie (Regensburg, Germany). After immunization with albumin-AGE:23, antibodies were affinity purified on human Aβ(1-40) conjugated to EMD-Epoxy activated beads (Merck, Darmstadt, Germany). Polyclonal mouse anti-AGE antibody was obtained after immunization with albumin-AGE:23 and human Aβ(1-40), in a molar ratio of 9:1. Polyclonal serum was obtained using standard immunization procedures, which were performed by the Academic Biomedical Cluster Hybridoma Facility (Utrecht University, The Netherlands). Subsequently monoclonal antibodies were generated using standard procedures.

ELISA: Binding of Antibodies Against Amyloid Peptides or Glycated Protein to Protein-AGE and Amyloid Fibrils

For ELISA's, amnyloid compounds were immobilized on Exiqon peptide or protein Immobilizers (Vedbaek, Denmark), as described before. Anti-AGE antibodies and commercially available anti-Aβ(1-42) H-43 (Santa Cruz Biotechnology, Santa Cruz, Calif., USA) were diluted in PBS with 0.1% v/v Tween20. Rabbit anti-human vitronectin K9234 was a kind gift of Dr. H. de Boer (UMC Utrecht), and was used as a negative control. For ELISA's with mouse polyclonal anti-albumin-AGE/Aβ, control serum with antibody elicited against an unrelated protein was used. Binding of mouse polyclonal anti-albumin-AGE/Aβwas performed using a dilution series of serum in PBS/0.1% Tween20. For competitive binding assays with IAPP, anti-AGE1 was pre-incubated with varying IAPP concentrations. The IAPP fibrils were spun down and the supernatant was applied in triplicate to wells of an ELISA plate coated with Aβ. Competitive binding assays with multiligand cross-β structure binding serine protease tPA were performed in a slightly different way. Coated Aβ and IAPP are overlayed with a anti-AGE1 or anti-Aβ(1-42) H-43 concentration related to the kD, together with a concentration series of tPA. A 10⁷-10⁴ times molar excess of lysine analogue εACA (10 mM) was present in the binding buffer in order to avoid binding of tPA to lysine residues of Aβ and IAPP, which would be independent of the presence of amyloid structures.

Pull-down Assay with Amyloid Peptides and Rabbit Anti-AGE1 Antibody

Anti-AGE1 was incubated with amyloid aggregates of Aβ(16-22), Aβ(1-40) and IAPP. After centrifugation, pellets were washed three times with PBS/0.1% Tween20, dissolved in non-reducing sample buffer (1.5% (m/v) sodium dodecyl sulphate, 5% (v/v) glycerol, 0.01% (m/v) bromophenol blue, 30 mM Tris-HCl pH 6.8). Supernatant after pelleting of the amyloid fibrils was diluted 1:1 with 2× sample buffer. Samples were applied to a polyacrylamide gel and after Western blotting, anti-AGE1 was detected with SWARPO.

Immunohistochemical Analysis of the Binding of Anti-AGE2 to an Amyloid Plaque in a Section of a Human Brain Inflicted by AD.

Rabbit anti-AGE2, affinity purified on an Aβ column, was used for assaying binding properties towards amyloid plaques in brain sections of a human with AD. The procedure was performed essentially as described above. To avoid eventual binding of 11 μg ml⁻¹ anti-AGE2 to protein-AGE adducts or to human albumin in the brain section, 300 nM of g6p-glycated dipeptide Gly-Lys was added to the binding buffer, together with 0.3% m/v BSA. After the immunohistochemical stain, the section was stained with Congo red.

Sandwich ELISA for Detection of Amyloid Albumin-AGE in Solution

For detection of amyloid cross-β structure in solutions we used the sandwich ELISA approach. Actilyse tPA was immoblized at a concentration of 10 μg ml⁻¹ to wells of a 96-wells protein Immobilizer plate (Exiqon, Vedbaek, Denmark). Concentration series of albumin-AGE:23 and albumin-control:23 were added to the tPA-coated wells, as well as to non-coated control wells. Binding of amyloid structures was detected upon incubation with 1 μg ml⁻¹ anti-Aβ(1-42) H-43 (Santa Cruz Biotechnology, Santa Cruz, Calif., USA) and subsequently 0.3 μg ml⁻¹ SWARPO, followed by ortho-phenylene-diamine/H₂O₂/H₂SO₄ stain.

Preparation of Cross-β Structure Rich Compounds

Soluble endostatin produced in the yeast strain Pichia pastoris was kindly provided by Dr. Kim Lee Sim (EntreMed, Inc., Rockville, Mass., USA). Cross-β structure rich endostatin was prepared from soluble endostatin as follows. Soluble yeast endostatin was dialysed overnight in 8 M urea and subsequently three times against H₂O. Endostatin precipitates as a fine white solid. The presence of cross-β structure was established by Congo red binding and X-ray fiber diffraction (Kranenburg, Bouma et al., 2002; Kranenburg, Kroon-Batenburg et al., 2003). Aggregated endostatin was solubilized in 0.1% formic acid, lyophilized fibrin peptides and Aβ were dissolved in H₂O and glycated albumin was extensively dialysed against water. Samples were taken up in a glass capillary. The solvent was then allowed to evaporate over a period of days. Capillaries containing the dried samples were placed on a Nonius kappaCCD diffractometer (Bruker-Nonius, Delft, The Netherlands). Scattering was measured using sealed tube MoKa radiation with a graphite monochromator on the CCD area detector during 16 hours. Scattering from air and the glass capillary wall were subtracted using in-house software (VIEW/EVAL, Dept. of Crystal- and Structural Chemistry, Utrecht University, The Netherlands). For preparation of advanced glycation end-product modified bovine serum albumin (albumin-AGE), 100 mg ml⁻¹ of albumin was incubated with PBS containing 1 M of glucose-6-phosphate (g6p) and 0.05% m/v NaN₃, at 37° C. in the dark. Glycation was prolonged up to 23 weeks (Bouma, Kroon-Batenburg et al., 2003). Human haemoglobin (Hb) at 5 mg ml⁻¹ was incubated for 32 weeks at 37° C. with PBS containing 1 M of g6p and 0.05% m/v of NaN₃. In control solutions, g6p was omitted. After incubations, solutions were extensively dialyzed against distilled water and, subsequently, stored at 4° C. Protein concentrations were determined with Advanced protein-assay reagent ADV01 (Cytoskeleton, Denver, Colo., USA). Glycation and formation of advanced glycation end-products (AGE) was confirmed by measuring intrinsic fluorescent signals from advanced glycation end-products; excitation wavelength 380 nm, emission wavelength 435 nm. In addition, binding of AGE-specific antibodies was determined. Presence of cross-β structure in albumin-AGE was confirmed by Congo red binding, Thioflavin T binding, the presence of β-sheet secondary structure, as observed with circular dichroism spectropolarimetry (CD) analyzes, and by X-ray fiber diffraction experiments (Bouma, Kroon-Batenburg et al., 2003). Presence of cross-β structure in Hb-AGE was conformed by tPA binding, CD analyzes, transmission electron microscopy imaging of fibrillar structures and by Congo red fluorescence measurements. Amyloid preparations of human γ-globulins were made as follows. Lyophilized γ-globulins (Sigma-Aldrich) was dissolved in a 1(:)1 volume ratio of 1,1,1,3,3,3-hexafluoro-2-propanol and trifluoro-acetic acid and subsequently dried under an air stream. Dried γ-globulins was dissolved in H₂O to an end-concentration of 1 mg/ml and kept at room temperature for at least three days. Aliquots were stored at −20° C. and analyzed for the presence of cross-β structure. Fluorescence of Congo red and Thioflavin T was assessed as well as tPA binding in an ELISA and tPA activating properties in the chromogenic plasmin assay.

Other peptide batches with amyloid-like properties were prepared as follows. Human Aβ(1-40) Dutch type (DAEFRHDSGYEVHHQKLVFFAQDVGSNKGAIIGLMVGGVV), islet amyloid polypeptide (IAPP, KCNTATCATQRLANFLVHSSNNFGAILSSTNVGSNTY), amyloid fragment of transthyretin (TTR11, YTIAALLSPYS) (Jaroniec, MacPhee et al., 2002), laminin α1-chain(2097-2108) amyloid core peptide (LAM12, AASIKVAVSADR) (Yamada, Kadoya et al., 2002), mouse non-amyloidogenic IAPP(20-29) core (mIAPP, SNNLGPVLPP), non-amyloid fragment FP10of human fibrin a-chain(148-157) (KRLEVDIDIK) (Schielen, Adams et al., 1191; Kranenburg, Bouma et al., 2002) and human fibrin α-chain(148-160) amyloid fragment with Lys157Ala mutation (FP13, KRLEVDIDIAIRS) (BB, unpublished and (Schielen, Adams et al., 1991; Kranenburg, Bouma et al., 2002)). For pull-down experiments Aβ and IAPP were dissolved in PBS, at 1 mg ml⁻¹ and kept at room temperature for at least three weeks. Alternatively, amyloid Aβ, IAPP, FP13 and LAM12 were disaggregated in a 1:1 (v/v) mixture of 1,1,1,3,3,3-hexafluoro-2-isopropyl alcohol and trifluoroacetic acid, air-dried and dissolved in H₂O (Aβ, IAPP, LAM12: 10 mg ml⁻¹, FP13: 1 mg ml⁻¹). After three days at 37° C., peptides were kept at room temperature for two weeks, before storage at 4° C. Freshly dissolved Aβ (10 mg ml⁻¹) in 1,1,1,3,3,3-hexafluoro-2-isopropyl alcohol and trifluoroacetic acid was diluted in H₂O prior to immobilization on ELISA plates. TTR11 (15 mg ml⁻¹) was dissolved in 10% (v/v) acetonitrile in water, at pH 2 (HCl), and kept at 37° C. for three days and subsequently at room temperature for two weeks. mIAPP and FP10 were dissolved at a concentration of 1 mg ml⁻¹in H₂O and stored at 4° C. Peptide solutions were tested for the presence of amyloid conformation by Thioflavin T- (ThT) or Congo red fluorescence as described (Hoppener, Oosterwijk et al., 1999; Hoppener, Ahren et al., 2000; Bouma, Kroon-Batenburg et al., 2003). ThT and Congo red fluorescence was enhanced for amyloid peptides, and not for non-amyloid mIAPP, FP10 or freshly dissolved Aβ.

Plasmin-α2-anti-plasmin- and Factor XIIa Measurements

Factor XIIa and PAP levels in citrated plasma of 40 apparently healthy controls and of 40 patients with systemic amyloidosis were measured. Factor XIIa was measured with an ELISA (Axis-Shield Diagnostics, Dundee, UK). PAP complexes were measured with the ELISA of Technoclone (Vienna, Austria). The control group consisted of 19 male and 21 female subjects with an average age of 49.4 years (standard deviation 6.8 years). The patient population consisted of 17 male and 23 female subjects with an average age of 51.8 years (standard deviation 9.9 years). Patients diagnosis was biopsy proven. All patients have provided informed consent prior to inclusion in this study and the study was approved by the local ethical committee.

Cloning and Expression of Recombinant Fibronectin Type I Domains

Amino-acid sequences of recombinantly produced domains of tPA, fibronectin and factor XII, and the domain architecture of the recombinant constructs are depicted in FIG. 25. Amino-acid residue numbering is according to SwissProt entries. Each construct has a carboxy terminal GST-tag (GST). Factor XII fibronectin type I domain (F) and fibronectin F4-5 are preceded by two amino acids (GA), following the C-terminus of the tPA propeptide. All F constructs are followed by the (G)RP sequence derived from the original pMT2-GST vector. For each recombinant construct, the oligonucleotides that were used for PCR are listed in FIG. 25. The relevant restriction sites are underlined. The tPA fibronectin type I domain (F, finger domain), together with the tPA propeptide, was amplified using 1 ng vector Zpl7 containing tPA (Johannessen, Diness et al., 1990) and oligonucleotides 1 and 2, digested with SalI and NotI and cloned into pMT2SM-GST (Gebbink, Zondag et al., 1995). As a result Schistosoma japonicum glutathion-S-transferase (GST) is fused to the C-terminus of the expressed constructs. The constructs were subsequently ligated with SalI and EcoRI in pGEM3Zf(−) (Promega, Madison, Wis., USA). The resulting plasmid was used as a cloning cassette for preparation of factor XII F and fibronectin F4-5 constructs. The selection of fibronectin type I domains of fibronectin was based on the following reasoning. tPA binds to fibrin with its fibronectin type I domain (van Zonneveld, Veerman et al., 1986) and competes with fibronectin for fibrin binding (Beckmann, Geiger et al., 1991). A fibrin binding-site of fibronectin is enclosed in its fibronectin type I 4-5 (Rostagno, Schwarzbauer et al., 1999). We show here that the fibronectin type I domain of tPA mediates binding to amyloid. This suggests that also the fibrin-binding fibronectin type I domains of fibronectin can bind to amyloid. All domains were cloned after the tPA propeptide using a BglII restriction site that is present between the tPA propeptide region and the F domain (Johannessen, Diness et al., 1990), and the NotI or KpnI site that is present in front of the thrombin cleavage site (Gebbink, Zondag et al., 1995). Subsequently, constructs were ligated HindIII and EcoRI in the pcDNA3.1 expression vector (Invitrogen, The Netherlands). This results in e.g., pcDNA3.1-factor XII F-GST and pcDNA3.1-Fn F4-5-GST. In addition, the GST tag alone, preceded by the tPA propeptide, was cloned into pcDNA3.1. The separate GST-tag has five additional residues at the N-terminus (GARRP). tPA cDNA was a kind gift of M. Johannessen (NOVO Research Institute, Bagsvaerd, Denmark). The cDNA encoding for factor XII was a kind gift of F. Citarella (University of Rome “La Sapienza,” Italy). S. A. Newman (New York Medical College, Valhalla, USA) kindly provided the cDNA encoding for an N-terminal fragment of human fibronectin, comprising fibronectin type I domains 4-5.

Alternatively, recombinant finger domains of fibronectin (F4-5) and tPA were expressed with a His-tag. Two fibronectin F4-5 constructs were cloned. One construct comprising the Igₖ signal sequence (vector 71, ABC-expression facility, Utrecht University/UMC Utrecht). With two designed primers (8, 9, see FIG. 25) the fibronectin fragment was obtained from the construct pcDNA3.1-Fn F4-5-GST and BamHI and NotI restriction sites were introduced at the termini. In addition, cDNA encoding for a C-terminal His-tag was included in the designed primer. The cDNA fragment was cloned BglII-NotI in vector 71 that was digested with BamHI-NotI. Vector 71 has a BamHI site next to the Ig,, signal sequence. See FIG. 25 for the construct details. A construct comprising the signal sequence of human growth hormone, the cDNA encoding for growth hormone (GH), an octa-His tag, a TEV cleavage site, the tPA F insert and a C-terminal hexa-His tag was made using vector 122b (ABC-expression facility). The tPA F-His cDNA was obtained using pcDNA3.1-tPA-F-GST as a template for a PCR with primers 10 and 11 (FIG. 25). The PCR insert was digested BglII-NotI, the vector was digested BamHI-NotI. The BamHI site is located next to the GH-His-TEV sequence. A second Fn F4-5 construct was made similarly to the GH-His-tPA F-His construct (See FIG. 25). tPA/Plasminogen activation assay and factor XII activation assay.

Plasmin activity was assayed as described (Kranenburg, Bouma et al., 2002). Peptides and proteins that were tested for their stimulatory ability were regularly used at 100 μg ml⁻¹. The tPA and plasminogen concentrations were 200 pM and 1.1 μM, respectively. Chromogenic substrate S-2251 (Chromogenix) was used to measure plasmin activity. Conversion of inactive zymogen factor XII to proteolytically active factor XII (factor XIIa) was assayed by measurement of the conversion of chromogenic substrate Chromozym-PK (Roche Diagnostics, Almere, The Netherlands) by kallikrein. Chromozym-PK was used at a concentration of 0.3 mM. Factor XII, human plasma prekallikrein (Calbiochem) and human plasma cofactor high-molecular weight kininogen (Calbiochem) were used at concentrations of 1 μg ml⁻¹. The assay buffer contained HBS (10 mM HEPES, 4 mM KCl, 137 mM NaCl, 5 μM ZnCl₂, 0.1% m/v BSA (A7906, Sigma, St. Louis, Mo., USA), pH 7.2). Assays were performed using microtiter plates (Costar, Cambridge, Mass., USA). Peptides and proteins were tested for their ability to activate factor XII. 150 μg ml⁻¹ kaolin, an established activator of factor XII was used as positive control and solvent (H₂O) as negative control. The conversion of Chromozym-PK was recorded kinetically at 37° C. for 60 minutes. Assays were done in duplicates. In control wells factor XII was omitted from the assay solutions and no conversion of Chromozym-PK was detected. In some assays albumin was omitted from the reaction mixture. In another type of factor XII activation assay, chromogenic substrate S-2222 (Chromogenix) was used to follow the activity of factor XII itself. With S-2222, activation of factor XII in plasma was established, using 60% v/v plasma, diluted with substrate and H₂O with or without potential cofactor. Furthermore, auto-activation of factor XII was established by incubating 53 μg/ml purified factor XII in 50 mM Tris-HCl buffer pH 7.5 with 1 mM EDTA and

and H₂O with or without potential cofactor.

Binding of tPA, Factor XII and the Fibronectin Type I Domains Thereof to Cross-β Structure Containing Protein Aggregates

Previously, we demonstrated that tPA specifically binds to any protein or peptide, as long as ligands have adopted amyloid-like cross-β structure conformation (Kranenburg, Bouma et al., 2002). Moreover, binding of tPA to aggregates with cross-β structure is accompanied by activation of tPA. Similar binding- and activation characteristics are indicated for factor XII, a serine protease with a similar domain architecture as tPA. Binding of factor XII to protein aggregates with cross-β structure is analyzed in an ELISA set-up. Activation of factor XII by aggregates comprising cross-β structure is analyzed according to the procedure described below. Next, binding of the fibronectin type I domain (F) of tPA and factor XII to amyloid-like aggregates was determined with ELISA's. Aggregates with cross-β structure are immobilized on Exiqon (Dahlback, Denmark) or Nunc (amino strips, catalogue #076901) Immobilizer plates, or Greiner microlon high-binding plates. Binding of tPA or factor XII was detected with specific antibodies; monoclonal 374b (American diagnostica) for tPA and polyclonal anti-factor XII antibody (Calbiochem). Binding of F domains was determined with F domains comprising a biotin tag, glutathione S transferase tag or His₆-tag. F domains were obtained as described below. In control experiments, binding of K2P tPA, a tPA analogue that lacks the N-terminal F-EGF-like domain-kringle 1 domain (Reteplase, Boehringer-Ingelheim, Germany), was tested. Binding of tPA and K2P tPA was tested in the presence of 10 mM ε-amino caproic acid (εACA), a lysine analogue that abolishes the binding of the tPA kringle2 domain to solvent exposed lysine residues.

Thioflavin T Fluorescence

Fluorescence of Thioflavin T (ThT)-protein/peptide adducts was measured as follows. Solutions of 25 μl/ml of protein or peptide preparations were prepared in 50 mM glycine buffer pH 9.0 with 25 μM ThT. Fluorescence was measured at 485 nm upon excitation at 435 nm. Background signals from buffer, buffer with ThT and protein/peptide solution without ThT were subtracted from corresponding measurements with protein solution incubated with ThT. Regularly, fluorescence of Aβ was used as a positive control, and fluorescence of FP10, a non-amyloid fibrin fragment (Kranenburg, Bouma et al., 2002), was used as a negative control. Fluorescence was measured in triplicate on a Hitachi F-4500 fluorescence spectrophotometer (Ltd., Tokyo, Japan).

Thioflavin T fluorescence was determined for human blood platelets, that were isolated as described above. The washed platelets in HEPES-Tyrode buffer (200,000/μl) were kept at room temperature. ThT fluorescence was determined at t=0 and at t=72 h, after storage at room temperature. For the measurements the platelets were diluted in assay buffer. Appropriate background signal readings were measured and subtracted.

Effects of Protein Aggregates with Cross-β Structure Conformation on the p38MAPK Pathway

Freshly isolated human blood platelets were obtained following the procedure described below. Seventy five μl of the platelet stock was added to 25 μl of agonist solution and incubated at room temperature for 1 minute and 5 minutes. Cells were fixed with 3% v/v formaldehyde and incubated on ice for 15 minutes. Platelets were pelleted upon centrifugation for 1 minute at 8450*g, and pellets were resuspended in 60 pI reducing sample buffer. After 6 minutes, at 100° C. samples were applied to SDS-PA gels for finally Western blot analysis. Blots were first incubated with polyclonal anti-p38MAPK antibody (Cell Signalling Technology) and SWARPO. Then, blots were also incubated with monoclonal anti-actin antibody AC40 (Sigma) and RAMPO, for scaling purposes. The relative degree of p38MAPK phosphorylation was determined using densitometric analysis of the blots.

Influence of Protein Aggregates with Cross-β Structure Conformation on Blood Platelet Aggregation

The influence of protein and peptide aggregates with cross-β structure conformation on blood platelet aggregation was tested with washed platelets in an aggregometric assay. Freshly drawn human aspirin free blood was mixed gently with citrate buffer to avoid coagulation. Blood was spinned for 15 minutes at 150*g at 20° C. and supernatant was collected; platelet rich plasma (PRP). Buffer with 2.5% trisodium citrate, 1.5% citric acid and 2% glucose, pH 6.5 was added to a final volume ration of 1:10 (buffer-PRP). After spinning down the platelets upon centrifugation for 15 minutes at 330*g at 20° C., the pellet was resuspended in HEPES-Tyrode buffer pH 6.5. Prostacyclin was added to a final concentration of 10 ng/ml, and the solution was centrifuged for 15 minutes at 330*g at 20° C., with a soft brake. The pellet was resuspended in HEPES-Tyrode buffer pH 7.2 in a way that the final platelet number was adjusted to 200,000/μl. Platelets were kept at 37° C. for at least 30 minutes, before use in the assays, to ensure that they were in the resting state.

For the aggregometric assays, 400 μl platelet solution was added to a glass tube with 100 μl containing the agonist of interest, fibrinogen and CaCl₂. Final concentrations of fibrinogen and CaCl₂ were 0.5 mg/ml and 3 mM, respectively. A stirring magnet was added and the apparatus (Whole-blood aggregometer, Chrono-log, Havertown, Pa., USA) was blanked. Aggregation was followed in time by measuring the absorbance of the solution, that will decrease in time upon platelet aggregation. As a positive control, 0.5 U/ml thrombin was used. Aggregation was followed for 10 minutes.

Activation of tPA by β2-glycoprotein I and Binding of Factor XII and tPA to β2-glycoprotein I

Purification of β2-glycoprotein I (p2gpi) was performed according to established methods. Recombinant human β2gpi was expressed in insect cells and purified as described in de Laat et al. (de Laat, Derksen et al., 2004a). Plasma derived β2gpi as used in the factor XII ELISA, the chromogenic plasmin assay and in the anti-phospholipid syndrome antibody ELISA (see below), was purified from fresh human plasma as described in Horbach et al. (Horbach, van Oort et al., 1996). Alternatively, β2gpi was purified from either fresh human plasma or from frozen-thawed plasma on an anti-β2 gpi antibody affinity column(Horbach, van Oort et al., 1998).

Activation of tissue-type plasminogen activator (tPA) (Actilyse, Boehringer-Ingelheim) by β2gpi preparations was tested in a chromogenic plasmin assay (see above). 100 μg/ml plasma β2gpi or recombinant β2gpi were tested for their stimulatory co-factor activity in the tPA-mediated conversion of plasminogen to plasmin and were compared to the stimulatory activity of cross-β structure rich fibrin peptide FP13 (Kranenburg, Bouma et al., 2002).

Binding of purified human factor XII from plasma (Calbiochem) or of purified recombinant human tPA to β2gpi purified from human plasma or to recombinant human β2gpi was tested in an ELISA. Ten pg of factor XII or tPA in PBS was coated onto wells of a Costar 2595 ELISA plate and overlayed with concentration series of the two β2gpi preparations. Binding of β2 gpi was assessed with monoclonal antibody 2B2 (Horbach, van Oort et al., 1998).

From preparations of β2gpi purified from fresh plasma or purified from frozen-thawed plasma 33 μg was brought onto a 7.5% poly-acrylarnide gel. After Western blotting, the nitrocellulose was incubated with 1000× diluted anti-human factor XII antibody (Calbiochem) and subsequently 3000× diluted SWARPO (DAKO).

Purified β2gpi from human plasma (400 μg/ml final concentration) was incubated with 100 μM cardiolipin vesicles or with 250 μg/ml DXS500k. Fluorescence of β2gpi in buffer, cardiolipin or DXS500k in buffer, buffer and ThT alone, and of β2gpi-cardiolipin adducts and β2gpi-DXS500k adducts with or without ThT was recorded as described above.

Binding of Anti-β2gpi Autoantibodies from Antiphospholipid Syndrome Auto-immune Patients to Immobilized β2gpi is Inhibited by Recombinant β2gpi and not by Plasma Derived β2gpi

When plasma derived β2gpi is coated onto hydrophilic ELISA plates, anti-β2gpi autoantibodies isolated from plasma of antiphospholipid syndrome auto-immune patients can bind (de Laat, Derksen et al., 2004b) (data kindly provided by B. de Laat, UMC Utrecht). To study the influence of co-incubations of the coated β2gpi with antibodies together with plasma β2gpi or recombinant β2gpi, concentration series of β2gpi were added to the patient antibodies. Subsequently, binding of the antibodies to coated β2gpi was assayed.

Structural Analysis of Oxidized Low Density Lipoprotein

Low density lipoproteins (LDL) were isolated from fresh (<24 h) human plasma, obtained from the Dutch bloodbank, that was kept at 10° C. LDL was isolated essentially as earlier described. Plasma was centrifuged in an ultracentrifuge for three subsequent cycles. The LDL fraction was isolated and stored under N₂, at 4° C. Before experiments, native LDL (nLDL) was dialyzed overnight at 4° C. against 0.9% w/v NaCl. To obtain oxidized LDL (oxLDL) with varying degrees of oxidation, native was first dialyzed against 0.15 M NaCl solution containing 1 mM NaNO₃, overnight at 4 ° C. Then, nLDL was diluted to 3-5 mg/ml, and CuSO₄ was added to a final concentration of 25 μM and incubated at 37 ° C. The degree of oxidation was followed by measurement of diene-formation at λ=234 nm (Ultrospec 3000 Spectrophotometer (Pharmacia Biotech)). To stop the oxidation reaction, LDL was dialyzed against 0.15 M NaCl, 1 mM NaNO₃ and 1 mM EDTA.

In time, an increase of the oxidation of LDL, as measured by specific diene fluorescence at 243 nm, was completed with Thioflavin T fluorescence and Congo red fluorescence measurements. Fluorescence measurements were performed as described above. In addition, the ability of nLDL and oxLDL to induce tPA activation was tested in the chromogenic plasmin assay. For this purpose, 24% oxidized LDL was used. Finally, we tested the ability of oxLDL to activate factor XII in plasma, as determined by following the conversion of the substrate S-2222, that is cleaved when activated factor XII is present (see above). Activation assays were performed in the wells of 96-wells ELISA plates, at 37° C.

Binding of Amyloid-specific Dyes to a Fibrin Clot.

Pooled human plasma of healthy donors was clotted by adding either phospholipids, CaCl₂ and kaolin when aPTT's are concerned, or tissue factor rich thromboplastin and CaCl₂ when PT assays are concerned. APTT's and PT's are performed in the presence of concentration series of the amyloid-specific dyes Congo red, Thioflavin S (ThS) or Thioflavin T (ThT), accompanied by the appropriate buffer controls, i.e., Na₂SO₄ for Congo red and NH₄Cl for ThS and ThT. Coagulation velocities under influence of amyloid-specific dyes were measured, or the binding of the dyes was established by visual inspection or by use of direct-light microscopy or fluorescence microscopy.

### Results

1. Structure is Present in Fibrin and in Synthetic Peptides Derived from Fibrin.

We have demonstrated that a fibrin clot stains with Congo red (not shown) and exhibits Thioflavin T fluorescence (FIG. 2A), indicative of the presence of amyloid structure in a fibrin clot. Using Congo red staining (not shown), circular dichroism measurements and X-ray diffraction analysis we show that synthetic peptides derived from the sequence of fibrin adopt cross-β structure (FIG. 2B, C). These peptides were previously found to possess tPA-binding and tPA-activating properties.¹⁸ The presence of cross-β structure in these peptides was found to correlate with the ability to stimulate tPA-mediated plasminogen activation (FIG. 2D).

In conclusion, these data provide evidence for physiological occurrence/relevance for formation of cross-β structure and the role of this structural element in binding of tPA to fibrin.

2. Aβ Structure, Binds Plasmin(ogen) and tPA, Stimulates Plasminogen Activation, Induces Matrix Degradation and Induces Cell Detachment that is Aggravated by Plasminogen and Inhibited by CpB

To test whether tPA, plasminogen and plasmin bind Aβ we performed solid-phase binding assays. Aβ was coated onto plastic 96-well plates and binding of the peptide to either plasmin(ogen) or to tPA was assessed by overlaying the coated peptide with increasing concentrations of either tPA, plasminogen or plasmin. Binding was assessed using specific antibodies to either plasmin(ogen) or to tPA by performing ELISA. FIG. 3A shows that tPA binds to Aβ with a Kd of about 7 nM, similar to the Kd of tPA binding to fibrin.⁵⁵ In contrast, we find no detectable binding of plasminogen to Aβ (FIG. 3B). However, activated plasminogen (plasmin) does bind to Aβ, and does so with a Kd of 47 nM. The fact that (active) plasmin, but not (inactive) plasminogen binds to Aβ suggests that plasmin activity, and hence the generation of free lysines is important for binding of plasmin to Aβ. To test this we made use of the lysine analogue ε-aminocaproic acid (εACA) and tested binding of plasmin and tPA to Aβ in its presence. We show that the binding of plasmin to Aβ is completely abolished in the presence of εACA (FIG. 3D). In contrast, FACA has no effect on the tPA-Ap interaction (FIG. 3C). Thus, we conclude that plasmin binds to free lysines that were generated during the incubation period, presumably via its lysine-binding Kringle domain(s). In line with this, the Kd of plasminogen Kringle domain binding to free lysines in fibrin is similar to the Kd for plasmin binding to Aβ.

We investigated the kinetics of plasminogen activation in the absence and the presence of Aβ. As has been published before by Kingston et al.²⁴ we find that Aβ potently stimulates the activation of plasminogen by tPA (FIG. 4A). However, we find that the reaction proceeds with second-order, rather than with first-order kinetics. We considered the possibility that the generation of free lysines during the reaction was causing this phenomenon (see below). tPA-mediated plasmin generation has been implicated in neuronal cell death caused by ischemia or by excitotoxic amino-acids. Recent data suggest that plasmin can degrade Aβ and thereby prevents Aβ toxicity.^{56; 57} We found that 48 hours following the addition of Aβ to a culture of differentiated N1E-115 cells, the majority of cells have died and detached from the matrix (not shown). When added together with Aβ, plasmin (up to 100 nM) was unable to ameliorate Aβ-induced cell detachment. Even prolonged pre-incubations of Aβ with 100 nM plasmin did not affect Aβ-induced cell detachment (FIG. 4B). Subsequently we considered the possibility that plasmin generation may potentiate rather than inhibit Aβ-induced cell detachment and survival. To test this we exposed N1E-115 cells to suboptimal concentrations of Aβ and low concentrations of plasminogen for 24 hours. In the absence of Aβ, plasminogen has no effect on cell adhesion (FIG. 4C). However, plasminogen has a dramatic potentiating effect on Aβ-induced cell detachment. The minimal levels of plasminogen that are required to potentiate Aβ-induced cell detachment (10-20 μg/ml) are well below those found in human plasma (250 μg/ml). Plasmin mediated degradation of the extracellular matrix molecule laminin precedes neuronal detachment and cell death in ischemic brain. We tested whether Aβ-stimulated plasmin generation leads to laminin degradation. Cell detachment was accompanied by degradation of the extracellular matrix protein laminin (FIG. 4D).

We considered the possibility that the generation of free lysines during Aβ stimulated plasmin formation was responsible for the observed second order kinetics. To test this, we made use of carboxypeptidase B (CpB), an enzyme that cleaves of C-terminal lysine and arginine residues) and the CpB-inhibitor CPI. FIG. 5A shows that in the presence of CpB the generation of plasmin is greatly diminished. Furthermore, this effect depends on CpB activity as it is abolished by co-incubation with CPI. FIG. 5A also shows that CpB does not completely abolish Aβ-stimulated plasmin generation, but that the reaction proceeds with slow first-order kinetics. These data suggest that the (plasmin-mediated) generation of free lysines during the reaction is essential for efficient Aβ-stimulated plasmin generation, presumably by supporting plasminogen and tPA binding through interaction with their respective Kringle domains. A similar dependency on the generation of C-terminal lysines has been shown for efficient fibrin-mediated plasmin generation.⁵⁸ These results show that Aβ-stimulated plasmin formation is regulated by carboxypeptidase B in vitro. Thus, if cell detachment is the result of plasmin generation, CpB may affect Aβ-induced cell detachment and/or viability. We show that cell detachment induced by plasminogen and Aβ is completely prevented by co-incubation with CpB (FIG. 5B,C). This is accompanied by a complete inhibition of Aβ-stimulated plasmin formation, both in the medium and on the cells (FIG. 5D).

3. Endostatin can Form Amyloid Fibrils Comprising Cross-β Structure.

Using Congo red staining (not shown), X-ray diffraction analysis and TEM we have demonstrated the presence of cross-β structure in aggregated endostatin from Escherichia coli, as wells as from Pichia pastoris, and the ability of endostatin to form amyloid fibrils (FIG. 6A-B). We found that bacterial endostatin produced refelction lines at 4.7 Å (hydrogen-bond distance), as well as at 10-11 Å (inter-sheet distance). The reflection lines show maximal intensities at opposite diffraction angles. The fiber axis with its 4.7 Å hydrogen bond repeat distance is oriented along the vertical capillary axis. This implies that inter-sheet distance of 10-11 Å A is perpendicular to these hydrogen bonds. This is consistent with the protein being a cross-β sheet conformation with a cross-β structure. Intramolecular β sheets in a globular protein cannot cause a diffraction pattern that is ordered in this way. From the amount of background scattering it follows that only part of the protein takes part in cross-β structure formation. We found that the presence of cross-β structures in endostatin correlates with its ability to stimulate tPA-mediated plasminogen activation (FIG. 6C) and correlates with neuronal cell death (FIG. 6D).

Here we have demonstrated that endostatin is an example of a denatured protein that is able to stimulate the suggested cross-β pathway.

4. IAPP Binds tPA and Stimulates tPA-mediated Plasminogen Activation.

Amyloid deposits of IAPP are formed in the pancreas of type II diabetic patients.⁵⁹ IAPP can cause cell death in vitro and is therefore thought to contribute to destruction of β-cells that is seen in vivo, which leads to insufficient insulin production. IAPP forms fibrils comprising cross-β structure.⁶⁰

We tested whether IAPP could stimulate tPA-mediated plasminogen activation (FIG. 7). Indeed, similar to Aβ, IAPP can enhance the formation of plasmin by tPA.

5. Glycated Albumin Binds Thioflavin T and tPA, and Aggregates as Amyloid Fibrils Comprising Cross-β Structure.

It has been demonstrated that glycation of several proteins can induce or increase the ability of these proteins to bind tPA and stimulate tPA-mediated plasmin formation.^{22; 61} We here show that glycation of albumin with g6p not only confers high affinity tPA binding to albumin (FIG. 8A), but also leads to its ability to bind Thioflavin T (FIG. 8C). Binding of tPA can be competed with Congo red (FIG. 8B). In addition, binding of Thioflavin T to glycated albumin can be competed by tPA (FIGS. 8D,E). The fact that Congo red and/or Thioflavin T and tPA compete illustrates that they have, either the same, or overlapping binding sites.

Analyses with TEM of the g6p-modified albumin preparations revealed that after a four-weeks incubation amorphous albumin aggregates are formed (FIG. 8G), which exhibits a CD spectrum indicative for the presence of 7% of the albumin amino-acid residues in β-sheet (Table I). Prolonged incubation up to 23 weeks resulted in a switch to highly ordered sheet-like fibrous structures, with a length of approximately 500 nm and a diameter ranging from about 50 to 100 nm (FIG. 8H). These fibres showed an increase to 19% β-sheet, when analysed with CD spectropolarimetry (Table I). Albumin from a different batch, that was glycated in the same way, already showed bundles of fibrous aggregates after a two-weeks period of incubation (FIG. 8I), whereas an increase in β-sheet content is not detected with CD spectropolarimetry (Table I). In each bundle about ten separate linear 3-5-nm-wide fibres with a length of 200-300 nm can be identified. On top of each bundle regularly distributed spots are seen, with a diameter of approximately 5 nm. These spots may be accounted for by globular albumin molecules that are bound to the fibres, or alternatively, that are partly incorporated in the fibres. Aggregates were absent in control albumin (not shown) and no β-sheets were measured using CD spectropolarimetry (Table I). The fibrous structures obtained after two-weeks and 23-weeks periods of glycation enhance the fluorescence of Thioflavin T (ThT) in a similar way, whereas the amorphous precipitates obtained after four weeks hardly increased the fluorescent signal.

X-ray fibre diffraction analyses revealed that albumin-g6p (23 weeks) comprises a significantly amount of crystalline fibres (FIGS. 8J,L), whereas diffraction patterns of albumin-g6p (2 weeks) and albumin-g6p (4 weeks) show features originating from amorphous precipitated globular protein, very similar to the patterns obtained for albumin controls (FIG. 8K). For albumin-g6p (23 weeks), the 4.7 Å repeat corresponds to the characteristic hydrogen-bond distance between β-strands in β-sheets. The 2.3 and 3.3 Å repeats have a preferred orientation perpendicular to the 4.7 Å repeat (FIG. 8M). Combining the 2.3 and 3.3 Å repeats suggests that the fibre axis is oriented perpendicular to the direction of the hydrogen bonds, with a repeat of 6.8 Å. This dimension corresponds to the length of two peptide bonds and indicates that β-strands run parallel to the fibre axis. This implies that the albumin-g6p (23 weeks) structure is composed of cross-β structure consisting of packed β-sheets of hydrogen-bonded chains (FIG. 8N). A similar orientation is found in amyloid fibrils of the first predicted a-helical region of PrP^{c}.^{β}When the a-axis is 9.4 Å, or alternatively 4.7 Å, and the c-axis is 6.8 Å, the 2.5 and 6.0 Å repeats can only be indexed as (h k 1). This implies a highly ordered b-axis repeat, corresponding to the inter β-sheet distance. With a-axis and c-axis of 4.7, or 9.4 Å and 6.8 Å, respectively, the strong 3.8 Å repeat should be indexed as (2 0 1) or (1 0 1). Considering all observations it is clear that the albumin-g6p fibres (23 weeks) are built up by cross-β structures, a characteristic feature of amyloid fibrils.

These results show that due to incubation and/or modification with sugar moieties cross-β structures in albumin are formed that are able to support tPA binding.

6. Glycation of Haemoglobin Induces tPA Binding and Fibril Formation.

Incubation of human haemoglobin with g6p resulted in high-affinity tPA binding (FIG. 9A). Amorphous aggregated Hb-g6p adducts including fibrils were observed with TEM (FIG. 9B), whereas control Hb did not aggregate (not shown). Human Hb of diabetes mellitus patients has the tendency to form fibrillar aggregates, once more than 12.4% of the Hb is glycated (Table II).

7. Amyloid Albumin is Formed Irrespective of the Original Carbohydrate (Derivative)

From the above listed observations it is clear that modification of —NH₂ groups of albumin with g6p induces formation of amyloid cross-β structure. The next question we addressed was whether triggering of refolding of globular albumin into an amyloid fold was a restricted property of g6p, or whether amyloid formation occurs irrespective of the original carbohydrate or carbohydrate derivative used for AGE formation. Albumin solutions were incubated for 86 weeks at 37° C. with 1 M g6p, 250 mM DL-glyceraldehyde/100 mM NaCNBH₃, 1 M β-D-(−)-fructose, 1 M D(+)-glucose, 500 mM glyoxylic acid/100 mM NaCNBH₃, and corresponding PBS and PBS/NaCNBH₃ buffer controls. Glyceraldehyde and glyoxylic acid are carbohydrate derivatives that are precursors of AGE in Maillard reactions.^{63; 64} After 86 weeks albumin-glyceraldehyde and albumin-fructose were light-yellow/brown suspensions. Controls were colorless and clear solutions. Albumin-glucose and albumin-glyoxylic acid were clear light-yellow to light-brown solutions. Albumin-g6p:86 was a clear and dark brown solution. AGE formation was confirmed by autofluorescence measurements using AGE-specific excitation/emission wavelengths (not shown), binding of moab anti-AGE 4B5 (not shown) and binding of poab anti-AGE (not shown). As expected, albumin-glyoxylic acid did not show an autofluorescent signal due to the fact that (mainly) non-fluorescent carboxymethyl-lysine (CML) adducts are formed.⁶³

The autofluorescence data and the binding of AGE-specific antibodies listed above show that various carbohydrates and carbohydrate derivatives can lead to similar AGE structures. Using g6p as starting point for AGE formation, we showed that albumin adopted amyloid properties, similar to those observed in well-studied fibrils of Aβ and IAPP. Therefore, we tested for the presence of amyloid structures in the albumin-AGE adducts obtained with alternative carbohydrates and derivatives. We measured fluorescence of albumin-AGE—ThT solutions (FIG. 10J) and of air-dried albumin-AGE preparations that were incubated with Congo red (FIGS. 10A-I). Incubation of albumin with glyceraldehyde, glucose or fructose resulted in an increased fluorescent signal of ThT (FIG. 10J). After subtraction of background signals of ThT and buffer, no specific amyloid—ThT fluorescence was measured for albumin-glyoxylic acid and buffer controls. Albumin-g6p, albumin-glyceraldehyde and albumin-fructose gave a Congo red fluorescent signal similar to signals of Aβ and IAPP (FIGS. 10C-E,G-H). With albumin-glucose, a uniformly distributed pattern of fluorescent precipitates is observed (FIG. 10F). With albumin-glyoxylic acid and buffer controls hardly any staining is observed (FIGS. 10A-B,I). These ThT and Congo red fluorescence data show that, in addition to albumin-g6p, albumin-glyceraldehyde, albumin-glucose and albumin-fructose have amyloid-like properties. To further substantiate these findings we tested for binding of amyloid-specific serine protease tPA in an ELISA. The enzyme bound specifically to albumin-g6p, albumin-glyceraldehyde, albumin-glucose and albumin-fructose (FIGS. 10K-L) and to positive controls Aβ and IAPP, as was shown before.⁴⁹ No tPA binding is observed for albumin-glyoxylic acid and buffer controls.

From the ThT, Congo red and tPA data, it is clear that inducing amyloid properties in albumin is not an exclusive property of g6p. Apparently, a spectrum of carbohydrates and carbohydrate derivatives, comprising g6p, glucose, fructose, glyceraldehyde, and likely more, has the capacity to trigger the switch from a globular native fold to the amyloid cross-β structure fold, upon their covalent binding to albumin.

8. Analysis of Congo Red Binding and tPA Binding to Aβ.

It has been demonstrated that Aβ can bind tPA and Congo red. We show that the binding of tPA to Aβ can be competed by Congo red (FIG. 11). These results support our finding that structures in Aβ, fibrin and glycated albumin are similar and are able to mediate the binding to tPA.

9. Binding of Human FXII to Amyloid Peptides and Proteins, that Contain the Cross-β Structure Fold.

The graphs in FIG. 12 show that FXII binds specifically to all amyloid compounds tested. k_{D}'s for hAβ(1-40), FP13, albumin-AGE and Hb-AGE are approximately 2, 11, 8 and 0.5 μM, respectively. The data obtained with the competitive FXII—tPA ELISA show that tPA efficiently inhibits binding of FXII to amyloid (poly)peptides (FIG. 12). From these data we conclude that FXII and f.l. tPA compete for overlapping binding sites on hAβ(1-40). K2P-tPA does not inhibit FXII binding. Binding of FXII to albumin-AGE is also effectively abolished by tPA but not by K2P-tPA, similar to what was observed for hAβ(1-40). This indicates that FXII may bind in a similar manner to hAβ(1-40) and albumin-AGE. In addition, these data show that the first three domains of tPA (finger, EGF-like, kringle 1) seem to be involved in the inhibitory effect of fl. tPA on interactions between FXII and amyloid hAβ(1-40) or albumin-AGE. Using a dot-blot assay we tested binding of FXII to spotted amyloid hΔIAPP and hAβ(1-40). No binding of FXII was observed for negative controls PBS and MΔIAPP (FIG. 12). However, FXII specifically bound to hAβ(1-40), in agreement with an earlier report,⁶⁵ as well as to hΔIAPP (FIG. 12). These data, together with the ELISA data shown in FIG. 12A-F, suggest that FXII can bind to polypeptides that do not share amino-acid sequence homology, though which share the cross-β structure fold. This is in accordance with our recent data on interactions between tPA and polypeptides, that contain the amyloid-specific fold.

10. Binding of tPA to the Cross-β Structure Containing Molecules, Aβ and Glycated Albumin Requires the Presence of an N-terminal Region in tPA, which Contains the Finger Domain.

Several domains in tPA have been shown to mediate binding to fibrin or fibrin fragments.^{12; 53; 66; 67} However it is unknown which domain of tPA is needed for binding to Aβ or other cross-β structure-containing molecules. We show that a mutated tPA, termed reteplase, which lacks the N-terminal finger, EGF and kringle 1 domain (K2-tPA) is unable to bind cross-β structure comprising molecules (FIGS. 13A,B). These results suggest that the N-terminal region is required for binding of tPA to fibrils comprising cross-β structure.

Expression and Purification of tPA-F-GST and RPTP-GST

Purification of the GST-tagged constructs tPA-F-GST and RPTPIμ-GST(control) from 293T medium using glutathione coupled to Sepharose 4B beads resulted in single bands of approximately 35 kDa and 150 kDa, respectively (not shown). Traces of BSA, originating from the FCS used in the medium, were also present.

ELISA: Binding of tPA-F-GST and RPTP-GST to Human Aβ(1-40) and Glycated Albumin

In the ELISA, control tPA bound to both human Aβ(1-40) and albumin-g6p in the presence of excess εACA (FIG. 13C). This shows that in the assay used tPA is capable of binding to fibrous amyloids in a kringle2-independent manner. The tPA-F domain bound to human Aβ(1-40) and to albumin-g6p, whereas no binding was observed with RPTPμ-GST. Therefore, binding observed with tPA-F-GST is specific and does not originate from the GST tag. This result points to the tPA finger domain as a specific domain designed by nature for binding to cross-β structured amyloid fibrils.

We prepared cDNA constructs in pcDNA3 of the F, F-EGF, EGF, F-EGF-K1 and K1fragments of human tPA. Recombinant proteins with a C-terminal GST tag were expressed in BHK cells and secreted to the medium. Medium from BHK cells expressing the GST tag alone was used as a control. Conditioned medium was used for pull-down assays using Aβ and IAPP fibrils, followed by Western blot analyses. Efficient binding to Aβ is evident for all three tPA mutants that contain the finger domain, i.e., F-GST, F-EGF-GST and F-EGF-K1-GST (FIG. 13D). The K1-GST and EGF-GST constructs, as well as the GST tag alone remain in the supernatant after Aβ incubation. A similar pattern was obtained after IAPP pull-downs (not shown).

We compared binding of purified tPA F-EGF-GST, recombinant f.l. Actilyse tPA and a GST control to immobilized amyloid Aβ, amyloid fibrin fragment α₁₄₈₋₁₆₀ FP13, amyloid IAPP and to non-amyloid mΔIAPP control (FIGS. 13E-G). Full-length tPA and tPA F-EGF-GST bind to all three amyloid peptides; for Aβ k_{D}'s for tPA and F-EGF are 2 and 2 nM, respectively, for FP13 5 and 2 nM, for IAPP 2 and 13 nM. No binding to non-amyloid mΔIAPP is observed (FIG. 13E). GST does not bind to FP13 and IAPP, while some binding is detected to Aβ. This may reflect the small fraction of GST that bound to Aβ in the pull-down assay (FIG. 13D).

With immunohistochemical analysis we tested binding of the purified recombinant tPA F-EGF-GST construct to paraffin sections of human brain inflicted by AD. Presence of amyloid depositions was confirmed by the Dept. of Pathology (UMC Utrecht) using standard techniques. In our experiments, these amyloid depositions were located using Congo red fluorescence (FIGS. 13I,K,M). In FIGS. 13H and I, and in FIGS. 13J and K it is clearly seen that areas that are positive for Congo red binding coincides with areas that are positive for tPA F-EGF-GST binding. Control stain with GST does not show specific binding of the tag alone (FIGS. 13L-M).

At present, based on sequential and structural homology, next to tPA three proteins are known that contain one or more finger domains, i.e., HGFa (one F domain), FXII (one F domain, Fn (one stretch of six F domains, two stretches of three F domains). From the above listed results we concluded that the F domain of tPA plays a crucial role in binding of tPA to amyloid (poly)peptides. We hypothesized that the finger domain could be a general cross-β structure binding module. Presently 4 proteins, tPA, FXII, HGFa and fibroenctin, are known that contain a finger motif. FIG. 14A schematically depicts the localization of the finger module in the respective proteins. FIG. 14B shows an alignment of the human amino acid sequences of the finger domains in these four proteins. FIG. 14C shows a schematic representation of the 3-dimensional structure of the finger domain of tPA, and of the fourth and fifth finger domain of fibronectin. To test our hypothesis that finger domains in general bind amyloid we cloned the F domains of HGFa and FXII, as wells as the fourth and fifth F domain of Fn, which are known for their capacity to bind to fibrin.⁶⁸ Using a pull-down assay we show that Fn F4-GST and Fn F4-5-GST, as well as FXII F-GST and HGFa F-GST specifically bind to Aβ(FIGS. 13M-N) and IAPP (not shown). Fn F5-GST binds to Aβ to some extent, however it is extracted less efficiently form the medium and seems to be party released during the washing procedure of the amyloid pellet (FIG. 13M). No construct was left in the medium after extraction of positive control tPA F-EGF-GST, whereas no negative control GST was detected in the pellet fraction (not shown). These data show that binding to amyloid (poly)peptides is not a unique capacity of the tPA F domain, yet a more general property of the F domains tested. Moreover, these data indicate that observed binding of FXII to amyloid (poly)peptides, as shown in FIGS. 13A,H and by Shibayama et al.,⁶⁵ is regulated via the F domain.

11. Amyloid-binding Domain of tPA

The finger domain of tPA has been shown to be of importance for high-affinity binding to fibrin.^{12; 66} Our present results using reteplase (K2-P tPA) and F-tPA, F-EGF-tPA and F-EGF-K1-tPA indicate an important role for the N-terminal finger domain of tPA in binding to stimulatory factors other than fibrin. Thus far all these factors bind Congo red and contain cross-β structure. Furthermore, the binding site of fibronectin for fibrin has been mapped to the finger-domain tandem F4-F5.⁶⁸ It has been demonstrated that plasminogen activation by full-length tPA, in the presence of fibrin fragment FCB2, can be inhibited by fibronectin.⁶⁹ Taken together, these observations suggest that tPA and fibronectin compete, via their finger domain, for the same or overlapping binding sites on fibrin. Our data, now, show that the F4-5 domains of Fn bind to amyloid Aβ.

12. Binding of Anti-AGE Antibodies to Amyloid (poly)Peptides and Binding of Anti-Aβ to Protein-AGE Adducts

Recently, O'Nuallain and Wetzel⁷⁰ showed that antibodies elicited against a peptide with amyloid characteristics, can bind to any other peptide with similar amyloid properties, irrespective of amino-acid sequence. Based on these data and on our observations that tissue-type plasminogen activator and factor XII can bind to a family of sequence-unrelated polypeptides, that share the amyloid specific cross-β structure fold, we hypothesize that a broader class of proteins can display affinity towards this structural unit, rather than towards a linear or conformational epitope, built up by specific amino-acid residues. This hypothesis prompted us with the question whether antibodies elicited against albumin-AGE, that contains the amyloid cross-β structure fold, also display the broad-range specificity towards any (poly)peptide which bears this cross-β structure fold.

In an ELISA set-up α-AGE1, which was elicited against g6p-glycated albumin-AGE, binds to amyloid albumin-AGE:23 (K_{d}=66 nM) and Hb-AGE:32 (K_{d}=20 nM), as wells as to Aβ(1-40) (K_{d}=481 nM) and IAPP (K_{d}=18 nM) (FIGS. 15A-C). Negative controls were non-glycated albumin and Hb, non-amyloid peptide mouse ΔIAPP for IAPP and polyclonal anti-human vitronectin antibody α-hVn K9234 for Aβ. To test whether the same fraction of α-AGE1 binds to IAPP and Aβ, the antibody was pre-incubated with IAPP fibrils, followed by pelleting of the fibrils, together with the possible amyloid-binding fraction of α-AGE1. Binding of α-AGE1, left in the supernatant, to Aβ(1-40) was reduced (FIG. 15D). This indicates that the same fraction of α-AGE1 binds to IAPP and Ap(1-40). With a pull-down assay we assessed the binding of anti-AGE1 to amyloid peptides in an alternative way. After incubation of anti-AGE1 solutions with amyloid fibrils Aβ(16-22) (FIG. 15E; lane 1-2), Aβ(1-40) (FIG. 15E; lane 4-5) and IAPP (FIG. 15E; lane 6-7), and subsequent pelleting of the amyloid fibrils, the supernatant was completely depleted from α-AGE1 by Aβ(16-22). With IAPP approximately 50% of the antibody is found in the amyloid fraction, whereas less antibody is pelleted with Aβ(1-40). These data obtained in a complementary way again show that anti-AGE1 can bind to amyloid peptides, which share no amino-acid sequence homology with albumin-AGE:23, though which share the cross-β structure fold. In addition, the observation that binding of tPA to amyloid peptides inhibits binding of anti-AGEI, also indicates that anti-AGE1, like tPA, binds to the cross-β structure fold (FIG. 15F-G). The observation that tPA reduces anti-AGE1 binding to Aβ to a lesser extent than the reduction seen with IAPP, is putatively related to the higher number of anti-AGE1 binding sites on coated Aβ, when compared with IAPP (see FIGS. 15B-C), and to the higher affinity of tPA for IAPP (k_{D}=6 nM) than for Aβ (k_{D}=46 nM), when using Exiqon ELISA plates (not shown). The binding data together suggest that anti-AGE1 binds to this amyloid fold, irrespective of the (poly)peptide that bears the cross-β structure fold, which identifies anti-AGE1 as a member of the class of multiligand cross-β structure binding proteins.

Based on the above listed results obtained with anti-AGE1, we tested whether commercially available rabbit anti-human Aβ(1-42) H-43 also displays broad-range specificity towards any (poly)peptide with unrelated amino-acid sequence, though with amyloid characteristics. Indeed, with an ELISA we could show that H-43 not only binds to Aβ(1-40), but also to IAPP and albumin-AGE (FIG. 15H). In addition, binding of H-43 to immobilized IAPP was effectively diminished by tPA (FIG. 15I). These observations together show that anti-Aβ(1-42) H-43 can bind to other amyloid (poly)peptides in a way similar to multiligand cross-β structure binding protein tPA.

ELISA's with polyclonal mouse anti-albumin-AGE/Aβ show that the antibody not only binds to these antigens, but that it specifically binds to other amyloid peptides than those used for immunization (FIGS. 15J-L). Similar to the rabbit anti-AGE1 antibody and anti-Aβ(1-42) H-43, anti-albumin-AGE/Aβ displays affinity for the amyloid peptides tested, irrespective of amino-acid sequence. This suggests that also mouse anti-albumin-AGE/Aβ is a multiligand amyloid binding antibody.

Based on the amyloid binding characteristics of anti-AGE1, anti-Aβ(1-42) H-43 and anti-albumin-AGE/Aβ, we purified the amyloid-binding fraction of anti-AGE2, which is elicited against albumin-AGE:23, with Aβ fibrils irreversibly coupled to a column. This fraction was used for immunohistochemical analysis of a human brain section that is inflicted by Alzheimer's disease. In FIG. 15M it is clearly seen that the antibody binds specifically to the spherical amyloid deposition, indicated by the Congo red fluorescence, shown in FIG. 15N.

Our finding that anti-amyloid and anti-AGE antibodies display affinity for a broad range of sequentially unrelated (poly)peptides, as long as the cross-β structure fold is present, is in agreement with the recently published data by O'Nuallain and Wetzel⁷⁰ and Kayed et al.⁷¹ From several older reports in literature it can be distilled that anti-cross-β antibodies can be obtained. For example, cross-reactive antibodies against fibrin and Aβ and against Aβ and haemoglobin are described.^{72; 73} We indicated here that fibrinogen and haemoglobin-AGE adopt the cross-β structure fold, which suggests that the cross-reactivity observed for anti-Aβ antibodies was in fact binding of anti-cross-β structure antibodies to similar structural epitopes on Aβ, fibrinogen and haemoglobin.

Based on our results with the poly-clonal anti-AGE and amyloid antibodies we hypothesized that anti-cross-β structure antibodies could be obtained. We therefore fused the spleen of mice immunized with glycated BSA and Aβ with myeloma cells. We subsequently selected potential anti-cross-β structure antibodies by examining binding of hybridoma produced antibodies to glycated haemoglobin and IAPP. Using this procedure, we isolated a monoclonal antibody 3H7, that recognizes glycated haemoglobin as well as several peptides that contain the cross-β structure (FIG. 16). No binding was observed to unglycated haemoglobin or a synthetic peptide that does not form amyloid fibrils (mΔIAPP)

13. Sandwich ELISA: Fishing Amyloid Structures from Solution

Using a sandwich ELISA approach with coated tPA that was overlayed with amyloid albumin-AGE:23 in solution, followed by detection with broad-range anti-Aβ(1-42) H-43 (FIG. 17), we were able to detect cross-β structure containing proteins in solution.

It is herein disclosed that the three-dimensional structures of the tPA finger-domain^{74; 75} and the fibronectin finger-domains 4-5^{75; 76} reveals striking structural homology with respect to local charge-density distribution. Both structures contain a similar solvent exposed stretch of five amino-acid residues with alternating charge; for tPA Arg7, Glu9, Arg23, Glu32, Arg30, and for fibronectin Arg83, Glu85, Lys87, Glu89, Arg90, located at the fifth finger domain, respectively. The charged-residue alignments are located at the same side of the finger module. These alignments may be essential for fibrin binding.

Based on our observations, results and the herein disclosed similarities, we show that the same binding sites for tPA become present in all proteins that bind and activate tPA and that this binding site comprises cross-β structure.

Taken together, our data show that cross-β structure is a physiological relevant quarternary structure element which appearance is tightly regulated and which occurrence induces a normal physiological response, i.e., the removal of unwanted biomolecules. To our knowledge the existence of a general system, which we term “cross-β structure pathway” to remove unwanted biomolecules is, herein, dislcosed for the first time. Our results show that this mechanism is fundamental to nature and controls many physiological processes to protect organisms from induced damage or from accumulating useless or denatured biomolecules. If by whatever means deregulated, this system may cause severe problems. On the other hand, if properly used this system may be applicable for inducing cell death in specific target cells, like for example tumour cells.

### EXAMPLE 14

The fibrinolytic cascade and the contact activation cascade of the haemostatic system are triggered by protein aggregates: Tissue-type plasminogen activator and factor XII interact with protein aggregates comprising amyloid-like cross-β structure, via their fibronectin type I domain. tPA, factor XII, fibronectin and the fibronectin type I domains of tPA, factor XII and fibronectin bind to protein aggregates with cross-β structure conformation

Previously, we established that tissue-type plasminogen activator interacts with protein and peptide aggregates that comprise the cross-β structure conformation, a structural element found in amyloid-like polypeptide assemblies (Kranenburg, Bouma et al., 2002; Bouma, Kroon-Batenburg et al., 2003). Now, we expanded this analysis to other proteins that resemble tPA domain architecture and to separate domains of tPA. Binding of full-length tPA, factor XII and fibronectin, as well as of fibronectin type I (finger, F) domains of tPA and factor XII and F4-5 of fibronectin, to protein and peptide aggregates with cross-β structure conformation was analyzed in an ELISA. In FIG. 18 it is shown that the full-length proteins as well as the recombinant F domains bind specifically to cross-β structure rich compounds. Binding of tPA and factor XII was established for immobilized amyloid-β(1-40) (Aβ) with amyloid-like properties, fibrin peptide FP13, that encompasses the tPA activating sequence 148KRLEVDIDIKIR160 of the fibrin α-chain, TTR11, which is an 11 amino-acid residues peptide from transthyretin that forms cross-β structure, and LAM12, which is a 12 amino-acid residues peptide from laminin that forms cross-β structure (FIGS. 18A,B). Negative controls were freshly dissolved, monomerized Aβ and non-amyloid murine islet amyloid polypeptide (mIAPP). For fibronectin, human amyloid IAPP is depicted instead of LAM12 (FIG. 18C). The separate F domains also bind to aggregates with cross-β structure, as depicted for Aβ and tPA F in FIG. 18D, and for all aggregates and factor XII F and fibronectin F4-5 in FIGS. 18E and F. In addition, immobilized fibronectin F4-5 with a His-tag specifically captures glycated haemoglobin with amyloid-like properties in solution (FIG. 18G).

Activation of Factor XII and tPA by Protein Aggregates with Amyloid-like Cross-β Structure

Contacting factor XII to artificial negatively charged surfaces results in its activation, as measured by the conversion of prekallikrein to kallikrein, which can convert chromogenic substrate Chromozyme PK (FIG. 19). Now, we demonstrate that peptide aggregates with cross-β structure conformation, the protein conformation found in amyloid, also stimulate factor XII activation (FIG. 19). Moreover, we demonstrate that kaolin is able to stimulate factor XII activation only when a protein cofactor, e.g., albumin, is present in the assay buffer (FIG. 19C). Contacting another established factor XII activating surface, i.e., dextran sulphate 500,000 Da (DXS500k), with various proteins, including lysozyme, γ-globulins, whole plasma and factor XII itself, results in the introduction of amyloid-like properties in the proteins, e.g., activation of tPA (FIG. 19D), binding of Thioflavin T (FIGS. 19E-G) and binding of tPA (FIGS. 19H-K), indicative for the formation of cross-β structure in the protein aggregates after exposure to the negatively charged surface. We also tested the ability of protein aggregates with cross-β structure conformation to induce auto-activation of factor XII. For this purpose, purified factor XII was incubated with substrate S-2222 and either buffer, or 1 μg/ml DXS500k, 100 μg/ml FP13 K157G, 10 μg/ml Aβ(1-40) E22Q and 10 μg/ml Hb-AGE. All three amyloid-like aggregates are able to induce factor XII auto-activation (FIG. 19M). FP13 K157G and Hb-AGE have a potency to induce auto-activation that is similar to the established surface activator DXS500k, whereas the potency of the Aβ(1-40) E22Q is somewhat lower.

The aforementioned observations show that both the contact system and the fibrinolytic system are activated in conformational diseases, or amyloidoses. We measured activation of these systems in patients with systemic amyloidoses. Increased activation of the fibrinolytic system is detected by measuring the levels of plasmin in complex with its circulating inhibitor α2-anti-plasmin (PAP). Increased activity of the contact system is detected by measuring the levels of activated factor XII. Both PAP levels (3192 ng ml⁻¹ vs 217 ng ml⁻¹) and factor XIIa levels (3.1 ng ml⁻¹ vs 2.1 ng ml⁻¹) were elevated in patient plasma (FIGS. 18H,I). Twelve out of 16 patients with elevated factor XIIa levels also had elevated PAP levels. These measurements show that systemic amyloidosis is accompanied by increased levels of activated serine proteases, and disclose a role for amyloid in activation of tPA and factor XII in vivo.

Factor XII, tPA, Fibronectin and their Recombinant Fibronectin Type I Domains Interact with Aggregates Comprising Cross-β Structure

Like tPA, factor XII, fibronectin, tPA F domain, factor XII F domain and fibronectin F4-5 domains bind to peptide aggregates with cross-β structure conformation. In addition, a chemically synthesized F domain of tPA (T. Hackeng, Academic hospital Maastricht, The Netherlands) the fibronectin F10-12 domains and the hepatocyte growth factor activator F domain bind to amyloid-like cross-β structure rich aggregates (B. Bouma, data not shown). Moreover, like tPA (Kranenburg, Bouma et al., 2002; Reijerkerk, Mosnier et al., 2003; Bouma, Kroon-Batenburg et al., 2003), factor XII becomes activated by amyloid-like aggregates. This has not only been established in an indirect way by measuring activated kallikrein from prekalikrein upon activation of factor XII, but also in a direct way by measuring auto-activation of factor XII upon exposure to amyloid-like protein aggregates (see FIG. 19). Our data also indicate that several negatively charged surfaces, that are well known for their ability to activate factor XII, i.e., kaolin and DXS500k, need a protein cofactor to gain stimulatory capacities. Binding of Thioflavin T and tPA after exposure of proteins to DXS500k show that the protein aggregate cofactors adopt the cross-β structure conformation, that is essential for both the factor XII activation and the tPA activation.

Our data show that both the fibrinolytic cascade and the contact system of blood coagulation become activated by activation of tPA and factor XII via protein aggregates with amyloid-like cross-β structure conformation, respectively. Moreover, the presence of amyloid-like protein conformation in the circulation or elsewhere in the body is a risk factor for inducing pathological activation of the fibrinolytic cascade and/or the contact activation system.

Our data on factor XII activation allow for a further analysis of the role of cross-β structure in factor XII activation. For example, factor XII auto-activation by cross-β structure is analyzed by contacting purified factor XII to cross-β structure in the presence of a chromogenic substrate that is converted when factor XII is activated. In addition, the influence of cross-β structure binding proteins and compounds on the activation of factor XII in the presence of cross-β structure is studied. Our observation that both tPA and factor XII become activated by proteins that are contacted to DXS500k further show that the fibrinolytic cascade and the contact activation cascade of the haemostatic system is activated by a common mechanism, in which protein aggregates comprising amyloid-like cross-β structure play an initiating role.

Our data show increased levels of PAP and fxIIa in amyloidosis patients. These results show a role for amyloid-like protein aggregates in the activation of tPA and factor XII in the amyloidosis patients. These results also provide a molecular explanation for bleeding diathesis and coagulation abnormalities that are recognised complications in amyloidosis patients. Dysregulation of the fibrinolytic system and the contact system by amyloid underlies this pathology directly by amyloid-mediated activation of tPA and indirectly by factor XII mediated formation of bradykinin which releases tPA. In addition to a role for fibronectin type I domain-comprising tPA and factor XII in amyloidosis, also a role for HGFA and fibronectin in conformational diseases is clear. Incorporation of fibronectin in amyloid deposits serves a function in neurite outgrowth in Alzheimer's disease, and represents a defense mechanism against amyloid deposition by shielding of toxic structures and by inhibiting fibril extension. Hepatocyte growth factor (HGF, scatter factor), a physiological substrate of HGFA, is increased in the brain of AD patients and HGF levels in plasma are elevated in amyloid A amyloidosis and light-chain amyloidosis. Our data, now, show that fibronectin type I—amyloid interactions are important for these activities.

### EXAMPLE 15

Isolated blood platelets become activated via their p38MAPK pathway and aggregate upon exposure to polypeptides with cross-β structure conformation.

Blood platelets become activated and aggregate upon exposure to proteins with cross-β structure conformation, express amyloid-like structures and show increased binding of amyloid dye Thioflavin T upon aging.

Incubation of freshly isolated platelets with various compounds that contain cross-β structure conformation results in activation of the p38MAPK pathway, as determined by analysis of p38MAPK phosphorylation. Incubation of platelets with amyloid haemoglobin-AGE results in platelet activation similar to the positive control native low density lipoprotein after 1 minute (FIG. 20A). After 5 minutes, Hb-AGE shows a prolonged activation whereas p38MAPK is not phosphorylated by nLDL stimulation anymore (FIG. 20B). Incubation with control haemoglobin results in background levels of p38MAPK phosphorylation, similar to buffer. Amyloid peptides FP13 and Aβ already potently induce p38MAPK phosphorylation after 1 minute incubation (FIG. 20C), whereas amyloid denatured γ-globulins and transthyretin amyloid fragment TTR11 induce p38MAPK phosphorylation only after 5 minutes stimulation (FIG. 20D). In a blood platelet aggregometer the influence of amyloid FP13 and denatured γ-globulins was tested and compared to the effect of thrombin on aggregation. Negative controls were HEPES-Tyrode buffer and 200 μg/ml native γ-globulins. Both FP13 and denatured γ-globulins with amyloid-like conformation induce platelet aggregation in a dose dependent manner (FIG. 20E). In a separate experiment the influence of platelet aging upon storage at room temperature, on Thioflavin T binding was assayed. After 72 hours, Thioflavin T fluorescence was approximately doubled, showing an increase in the amount of cross-β structure (FIG. 20F).

Recent insights have indicated that the formation of amyloid is not necessarily the result of a defect in the normal folding or clearance pathway, but that amyloid is also formed through normal biological proteolytic processing. We found that (i) activation of platelets induce amyloid at their cell surface and (ii) that platelets adhered to von Willebrand Factor (vWF) or collagen surface under flow express amyloid domains (FIGS. 21A,B). Expression is at the cell body and areas of spreading are negative (vWF surface) and at tips of aggregates (collagen) suggesting that adhered and aggregated platelets express areas of rich and poor in amyloid. Platelets stimulated with TRAP (an activator of the PAR-1 receptor without proteolytic properties and incapable of converting released fibrinogen into fibrin) and thrombin (an activator of PAR-1 and PAR-4 through proteolysis and an activator of fibrin formation) express arnyloid as visualized using the fluorescent amyloid dyes Congo Red and ThT in a FACS analysis (FIGS. 21C,D). Resting platelets do not express arnyloid at the cell surface (FIG. 21C).

To test whether arnyloid may influence platelet aggregation by classical stimuli, we tested whether arnyloid specific dyes and the amyloid binding protein tPA affect platelet aggregation. Indeed, using optical aggregometry we observed that Congo Red, ThT as well as tPA inhibited platelet aggregation. Dose response studies show up to 30% inhibition by 200 ,μM Congo red and upto 45% inhibition by 200 μM ThT (FIG. 4E). tPA (1 μM) even induced 55% inhibiton of thrombin-induced aggregation. The inhibition persisted in platelets treated with indomethacin (aspirin-like) and AR-C6993MX (clopidogrel-like), indicating that amyloid contributed to platelet aggregation via mechanisms independent of thromboxane A₂ formation or P2Y12 stimulation through released ADP (FIG. 21F).

Cross-β Structure is Thrombogenic

Based on our observations that compounds which comprise cross-β structure induces activation in platelets of the p38MAPK pathway and induces platelet aggregation, we conclude that the exposure of platelets to proteins with cross-β structure in the circulation has similar effects. Platelets stored under conditions recommended by the blood bank show increased binding of an amyloid dye. Therefore, aging platelets themselves may contain proteins that have adopted the activating cross-β structure conformation. Isolating only those cells in donor blood which display relatively minimal ThT binding is beneficial for the acceptor of the platelets.

Our pilot studies have demonstrated the presence of amyloid on activated and adhered platelets. For the development of effective anti-thrombogenic agents, knowledge on which protein or proteins are forming amyloid-like structures on platelets is required. In addition, knowledge on which pathways are involved in extracellular exposure upon platelet activation of putatively intracellularly stored amyloid-like conformation rich proteins, is beneficial for the development of treatment strategies that prevent amyloid exposure on platelets. With this knowledge pathway inhibitors are developed solely or in addition to blockers/scavengers of amyloid-like aggregates.

### EXAMPLE 16

Relationship Between the Structure of β-glycoprotein I, the Key Antigen in Patients with Antiphospholipid Synsdrome, and Antigenicity.

The Anti-phospholipid Syndrome and Conformationally Altered β-glycoprotein I

The anti-phospholipid syndrome (APS) is an auto-immune disease characterized by the presence of anti-β2-glycoprotein I auto-antibodies (de Groot and Derksen, 2004; de Laat, Derksen et al., 2004a; de Laat, Derksen et al., 2004b). Two of the major clinical concerns of the APS are the propensity of auto-antibodies to induce thrombosis and the risk for fetal resorption (de Groot, Horbach et al., 1996; Connor and Hunt, 2003). Little is known about the onset of the auto-immune disease. Recent work has demonstrated the need for conformational alterations in the main antigen in APS, β-glycoprotein I (β2gpi), before the initially hidden epitope for auto-antibodies is exposed (Matsuura, Igarashi et al., 1994; de Laat, Derksen et al., 2004a; de Laat, Derksen et al., 2004b). Binding of native β2gpi to certain types of ELISA plates mimicks the exposure of the cryptic epitopes that are apparently present in APS patients. It has been demonstrated that anti-β2gpi auto-antibodies do not bind to globular β2gpi in solution, but only then when β2gpi has been immobilized to certain types of ELISA plates (Matsuura, Igarashi et al., 1994; de Laat, Derksen et al., 2004a; de Laat, Derksen et al., 2004b). Thus, the globular and native form of the protein is not the primary antigen in the autoimmune disease. Auto-antibodies seem to be elicited against a conformationally altered form of autologous β2gpi, which would fit in the “danger” model of immunology (Matzinger, 2002a; Matzinger, 2002b). This proposed mechanism could explain the observed risk for thrombosis in APS patients. Auto-antibodies prevent clearance of conformationally altered β2gpi with exposed amyloid cross-β structure epitopes. This induces activation of the contact system, platelet activation and tissue factor expression on endothelial cells.

Factor XII and tPA Bind to Recombinant β2gpi and to β2gpi Purified from Frozen-thawed Plasma, and not to β2gpi Purified from Fresh Plasma

Recombinant β2gpi and not β2gpi purified from fresh plasma, stimulates effectively the tPA-mediated conversion of plasminogen to plasmin, as measured as the conversion of the plasmin specific chromogenic substrate S-2251 (FIG. 22A). Factor XII and tPA do not bind to β2gpi purified from fresh human plasma (FIGS. 22B,C). Recombinant β2gpi, however, binds to factor XII with a k_{D} of 20 nM and to tPA with a k_{D} of 51 nM (FIG. 22). In addition, when β2gpi is purified from plasma that was frozen at −20° C. and subsequently thawed, factor XII co-elutes from the anti-β2gpi antibody affinity column, as shown on Western blot after incubation of the blot with anti-factor XII antibody (FIG. 22D). In FIG. 22E, the inhibitory effect of recombinant β2gpi on binding of anti-β2gpi auto-antibodies isolated from patients with anti-phospholipid syndrome to immobilized β2gpi is shown. It is shown that plasma derived β2gpi in solution has no effect on the antibody binding to immobilized β2gpi. FIG. 22F shows that exposure of β2gpi to cardiolipin or dextransulphate 500,000 Da introduces an increased ThT fluorescence signal, indicative for a conformational change in β2gpi accompanied with the formation of cross-β structure. Again, recombinant β2gpi initially gave a higher Thioflavin T fluorescence signal than native β2gpi purified from plasma. In addition, tPA binds with higher affinity and to a higher extent to β2gpi bound to immobilized cardiolipin, than to β2gpi that is directly immobilized on wells of an ELISA plate (B. de Laat, data not shown). These observations also show that cardiolipin has a denaturing effect, thereby inducing amyloid-like conformation in β2gpi, necessary for tPA binding. Binding of recombinant β2gpi and β2gpi purified from plasma to tPA has also been assessed in an alternative set-up. TPA was immobilized onto the wells of an ELISA plate and subsequently overlayed first with concentration series of recombinant β2gpi or of plasma β2gpi, and an anti-β2gpi antibody (FIG. 5G). In FIG. 5H we show that exposure of β2gpi to cardiolipin, immobilized on the wells of an ELISA plate, renders β2gpi with tPA binding capacity. Binding of β2gpi directly to the ELISA plate results in less tPA binding. These observations, together with the observation that exposure of β2gpi to cardiolipin vesicles induced ThT binding capacity (FIG. 22F), show that introducing β2gpi to a denaturing surface induces formation of amyloid-like cross-β structure conformation. In FIG. 20 we show that blood platelets are activated and aggregate upon exposure to protein aggregates with cross-β structure. Therefore, we tested whether recombinant β2gpi, that shows properties reminiscent to an aggregated with cross-β structure conformation, and β2gpi purified from human plasma, are able to induce platelet activation (FIG. 22I). Exposure of platelets to recombinant β2gpi results in somewhat higher phosphorylation of p38MAPK. From these results we conclude that β2gpi exposing the amyloid-like cross-β structure conformation may serve as an activator of platelets, resulting in platelet aggregation. In this way, β2gpi is turned into a thrombogenic protein, giving a rationale to the observed thrombogenic activity seen in patients with the APS.

Epitopes for Auto-antibodies are Specifically Exposed on Non-native Conformations of β2gpi Comprising Cross-β Structure

FIG. 22 shows that preparations of β2gpi react with amyloid cross-β structure markers. In addition, exposure of β2gpi to cardiolipin introduces tPA binding capacity (data not shown). The β2gpi preparations with cross-β structure conformation express epitopes that are recognized by anti-p2gpi auto-antibodies isolated from APS patient plasma. Furthermore, exposure of β2gpi to cardiolipin or dextran sulphate 500,000 Da induces an increased fluorescence when ThT is added, indicative for the formation of cross-β structure when β2gpi contacts a negatively charged surface. Interestingly, it has previously been observed that exposure of β2gpi to cardiolipin is a prerequisite for the detection of anti-β2gpi antibodies in sera of immunized mice (Subang, Levine et al., 2000). These combined observations point to a role for conformational changes in native β2gpi, necessary to expose new immunogenic sites. The cross-β structure element is part of this initially absent epitope. To further establish this view, immunization studies with native β2gpi and conformationally altered β2gpi, with or without cross-β structure, can be performed. Sources of conformationally altered β2gpi are recombinant β2gpi, or β2gpi exposed to any denaturing surface, e.g., cardiolipin and DXS500k. In vitro cellular assays and in vivo mouse models help to gain insight into the putative role of the cross-β structure in auto-immunity. Cross-reactivity of antibodies directed against conformationally altered β2gpi with certain pathogens add to the combined ideas that early episodes of pathogen infection induce anti-p2gpi auto-antibodies and to our newly disclosed hypothesis that anti-p2gpi auto-antibodies are raised against a form of β2gpi with cross-β structure. Antibodies against the pathogens are putatively directed to amyloid-like proteins, present at the surface (Gebbink et al., Nature Rev. Immunol. 2005, in press), which would then explain cross-reactivity with host proteins comprising cross-β structure, including β2gpi.

With our observations that support the idea that cross-β structure in part build up an epitope recognized by autoimmune antibodies, our studies are expanded to other diseases and complications in which auto-antibodies play a role. For example, haemophilia patients with anti-factor VIII auto-antibodies are screened for the presence of antibodies in their plasma that recognize the cross-β structure conformation. A more detailed analysis reveals whether putative cross-β structure binding antibodies specifically bind in part to cross-β structure in the antigen, or whether the antibodies bind to cross-β structure present in any unrelated protein.

### EXAMPLE 17

The Artherogenic Form of Low Density Lipoproteins, Oxidized LDL, Exhibits Amyloid-like Structural Properties

Oxidation of Low Density Lipoprotein Particles Contributes to the Pathogenesis of Artherogenesis

Misfolding and aggregation of the apoB protein fraction of LDL upon oxidation, its resistance to proteolysis and its cytotoxicity are motifs commonly seen for amyloid-like protein aggregates (Ursini, Davies et al., 2002). Moreover, multiligand receptors with affinity for amyloid-like structures, i.e., CD36, RAGE, scavenger receptor A and scavenger receptor B-I, are also receptors for oxidized adducts of lipoproteins (Horiuchi, Sakamoto et al., 2003; Korporaal, Gorter et al., 2005). Therefore, it has been suggested that the role of amyloid-like apoB in oxidized LDL particles during atherosclerosis is similar to the pathogenic role of amyloid-like aggregates in protein misfolding diseases.

Oxidized LDL Displays Amyloid-like Features Pointing to the Presence of Cross-β Structure Conformation

Freshly isolated low density lipoprotein (LDL) was oxidized upon incubation with 25 μM CuSO₄, for various incubation times. In time, the degree of oxidation was determined by reading the absorbance of diene structures at 234 nm, as well as the fluorescence upon incubation of oxidized LDL (oxLDL) with Congo red or Thioflavin T (FIGS. 23A,B). With a 24% oxidized oxLDL preparation, the ability to activate tPA in the chromogenic plasmin activation assay was determined and compared to native LDL. It was clearly seen that upon oxidation LDL gains tPA activating properties (FIG. 23C). In an additional experiment, the ability of oxidized LDL to activate factor XII in plasma was determined in a chromogenic assay using substrate S-2222. Like amyloid fibrin-derived peptide FP13 K157G, oxLDL stimulates the conversion of S-2222, indicative for the ability of oxLDL to induce factor XII activation (FIG. 23D).

The Pathological Role of oxLDL During Atherosclerosis is Related to the Presence of Amyloid-like Cross-β Structure Conformation in the apoB Fraction

Previous data show that oxLDL plays an important role in the pathological events seen during atherosclerosis. The role of the interaction between oxLDL particles and multiligand receptor CD36 on the surface of blood platelets has been demonstrated (Takahashi, Fuda et al., 1998). CD36 is one of the known cellular receptors with broad range ligand specificity, including specificity for amyloid-like structures (Coraci, Husemann et al., 2002; Bamberger, Harris et al., 2003). Our data, now, add to our idea that the cross-β structure conformation in oxLDL, as well as in amyloid-β (Bamberger, Harris et al., 2003) and in glycated proteins(Ohgami, Nagai et al., 2001; Bouma, Kroon-Batenburg et al., 2003), is the true ligand binding site, that is important in mediating signals outside-in the platelets. Activation of platelets by oxLDL results in platelet aggregation, that contributes to the thromogenic conditions seen during atherosclerosis. Our observations that amyloid-like protein aggregates with cross-β structure conformation are also able to activate platelets and to induce platelet aggregation fit in the idea that protein aggregates comprising amyloid-like structures, including oxLDL, play a pivotal role in the pathological conditions that come with thrombogenesis. Moreover, our data show that oxLDL contributes to pathological conditions via two additional ways. First, the fibrinolytic cascade is activated by inducing tPA activation. Second, oxLDL activates the contact system of blood coagulation, by inducing factor XII activation. The ability of oxLDL to both activate tPA and factor XII, is similar to what is observed with many of our amyloid-like peptides and proteins with cross-β structure. Therefore, this ability of oxLDL points to the presence of cross-β structure in the apoB protein part of the oxidized LDL particles.

### EXAMPLE 18

A Fibrin Clot Comprises Amyloid-like Cross-β Structure Conformation.

A Fibrin Clot Binds Amyloid-specific Dyes Congo Red, Thioflavin T and Thioflavin S.

Incubation of a preformed fibrin clot with the amyloid-specific dyes Congo red (CR), Thioflavin S (ThS) or Thioflavin T (ThT) results in specific binding of the dyes, as assayed with direct-light microscopy and fluorescence microscopy (FIGS. 24A,B). In addition, formation of a fibrin clot is delayed in the presence of the amyloid-specific dyes, as established both in aPTT's (FIGS. 24C-E) and in PT's (FIGS. 24F-H). These data indicate that a fibrin clot is composed of aggregates comprising the amyloid-specific cross-β structure conformation. Moreover, the data show that formation of a three-dimensional fibrin polymer network is dependent on cross-β structure formation, as introduction of amyloid binding dyes inhibit fibrin assembly into clots. Our previously disclosed observations (see above) showed that platelet activation and aggregation is induced by cross-β structure rich activators (FIG. 20). Moreover, we showed that platelet activation is accompanied by the surface exposure of amyloid-like aggregates of proteins with cross-β structure conformation (FIG. 21). Combined with the observation that a fibrin clot comprises cross-β structure conformation, it is now obvious that polymerized fibrin in a blood clot serves as the cross-β structure rich source for (further) platelet activation.

### Tables

TABLE I
Percentage β-sheet, as calculated from CD spectra

| | Incubation time | β-sheet |
|---|---|---|
| Sample^{‡} | (weeks) | (%)^{†} |
| Aβ(16-22) | | 100 |
| Albumin-glycerald. | 2 | 0 |
| Albumin control | 2 | 0 |
| Albumin-g6p | 2 | 0 |
| Albumin-g6p | 4 | 7 |
| Albumin control | 23 | 0 |
| Albumin-g6p | 23 | 19 |

^{‡}Two-weeks incubated albumin was from a different batch than four- and 23-weeks incubated albumin.
^{†}Percentage of amino-acid residues in β-sheets are given.

TABLE II
Correlation between Hb_{Alc} concentrations and Hb fibril formation in
vitro.

| Healthy controls | | | Diabetes mellitus patients | | |
|---|---|---|---|---|---|
| sample | [Hb_{Alc}] (%)^{‡} | Fibres^{†} | sample | [Hb_{Alc}] (%)^{‡} | Fibres^{†} |
| 1 | 5.6 | − | 1 | 5.5 | − |
| 2 | 5.9 | − | 2 | 5.8 | − |
| 3 | 6.2 | − | 3 | 5.8 | − |
| | | | 4 | 10.7 | − |
| | | | 5 | 11.3 | − |
| | | | 6 | 11.6 | − |
| | | | 7 | 12.4 | + |
| | | | 8 | 12.5 | − |
| | | | 9 | 12.5 | − |
| | | | 10 | 12.6 | + |
| | | | 11 | 12.7 | − |
| | | | 12 | 12.8 | − |
| | | | 13 | 13.3 | + |
| | | | 14 | 13.7 | + |
| | | | 15 | 14.8 | + |
| | | | 16 | 15.3 | + |

^{‡}The Hb_{Alc} concentration is given as a percentage of the total amount of Hb present in erythrocytes of diabetes mellitus patients and of healthy controls. The s.d. is 2.3% of the values given.
^{†}Presence of fibres as determined with TEM.

### REFERENCES

1. Bucciantini M, Giannoni E, Chiti F, et al. Inherent toxicity of aggregates implies a common mechanism for protein misfolding diseases. Nature. 2002;416:507-511.
2. Rochet J C, Lansbury P T, Jr. Amyloid fibrillogenesis: themes and variations. Curr Opin Struct Biol. 2000;10:60-68.
3. Carmeliet P, Bouche A, De Clercq C, et al. Biological effects of disruption of the tissue-type plasminogen activator, urokinase-type plasminogen activator, and plasminogen activator inhibitor-1 genes in mice. Ann N Y Acad Sci. 1995;748:367-381.
4. Carmeliet P, Collen D. Genetic analysis of the plasminogen and coagulation system in mice. Haemostasis. 1996;26 Suppl 4:132-153.
5. Collen D, Lijnen H R. Molecular basis of fibrinolysis, as relevant for thrombolytic therapy. Thromb Haemost. 1995;74:167-171.
6. Lijnen H R, Collen D. Mechanisms of physiological fibrinolysis. Baillieres Clin Haematol. 1995;8:277-290.
7. Bosma P J, Rijken D C, Nieuwenhuizen W. Binding of tissue-type plasminogen activator to fibrinogen fragments. Eur J Biochem. 1988;172:399-404.
8. Grailhe P, Nieuwenhuizen W, Angles-Cano E. Study of tissue-type plasminogen activator binding sites on fibrin using distinct fragments of fibrinogen. Eur J Biochem. 1994;219:961-967.
9. Nieuwenhuizen W. Fibrin-mediated plasminogen activation. Ann N Y Acad Sci. 2001;936:237-246.
10. Yakovlev S, Makogonenko E, Kurochkina N, Nieuwenhuizen W, Ingham K, Medved L. Conversion of fibrinogen to fibrin: mechanism of exposure of tPA- and plasminogen-binding sites. Biochemistry. 2000;39:15730-15741.
11. Bakker A H, Weening-Verhoeff E J, Verheijen J H. The role of the lysyl binding site of tissue-type plasminogen activator in the interaction with a forming fibrin clot. J Biol Chem. 1995;270:12355-12360.
12. de Munk G A, Caspers M P, Chang G T, Pouwels P H, Enger-Valk B E, Verheijen J H. Binding of tissue-type plasminogen activator to lysine, lysine analogues, and fibrin fragments. Biochemistry. 1989;28:7318-7325.
13. Fleury V, Loyau S, Lijnen H R, Nieuwenhuizen W, Angles-Cano E. Molecular assembly of plasminogen and tissue-type plasminogen activator on an evolving fibrin surface. Eur J Biochem. 1993;216:549-556.
14. Haddeland U, Sletten K, Bennick A, Nieuwenhuizen W, Brosstad F. Aggregated, conformationally changed fibrinogen exposes the stimulating sites for t-PA-catalysed plasminogen activation. Thromb Haemost. 1996;75:326-331.
15. Hasan A A, Chang W S, Budzynski A Z. Binding of fibrin fragments to one-chain and two-chain tissue-type plasminogen activator. Blood. 1992;79:2313-2321.
16. Nieuwenhuizen W, Vermond A, Voskuilen M, Traas D W, Verheijen J H. Identification of a site in fibrin(ogen) which is involved in the acceleration of plasminogen activation by tissue-type plasminogen activator. Biochim Biophys Acta. 1983;748:86-92.
17. Nieuwenhuizen W, Schielen W J, Yonekawa O, Tesser G I, Voskuilen M. Studies on the localization and accessibility of sites in fibrin which are involved in the acceleration of the activation of plasminogen by tissue-type plasminogen activator. Adv Exp Med Biol. 1990;281:83-91.
18. Nieuwenhuizen W. Sites in fibrin involved in the acceleration of plasminogen activation by t-PA. Possible role of fibrin polymerisation. Thromb Res. 1994;75:343-347.
19. Schielen W J, Adams H P, Voskuilen M, Tesser G I, Nieuwenhuizen W. The sequence A alpha-(154-159) of fibrinogen is capable of accelerating the t-PA catalysed activation of plasminogen. Blood Coagul Fibrinolysis. 1991;2:465-470.
20. Tsurupa G, Medved L. Identification and characterization of novel tPA- and plasminogen-binding sites within fibrin(ogen) alpha C-domains. Biochemistry. 2001;40:801-808.
21. Yonekawa O, Voskuilen M, Nieuwenhuizen W. Localization in the fibrinogen gamma-chain of a new site that is involved in the acceleration of the tissue-type plasminogen activator-catalysed activation of plasminogen. Biochem J. 1992;283 (Pt 1):187-191.
22. Bobbink I W, Tekelenburg W L, Sixma J J, de Boer H C, Banga J D, de Groot P G. Glycated proteins modulate tissue-plasminogen activator-catalyzed plasminogen activation. Biochem Biophys Res Commun. 1997;240:595-601.
23. Hu G F, Riordan J F. Angiogenin enhances actin acceleration of plasminogen activation. Biochem Biophys Res Commun. 1993;197:682-687.
24. Kingston I B, Castro M J, Anderson S. In vitro stimulation of tissue-type plasminogen activator by Alzheimer amyloid beta-peptide analogues. Nat Med. 1995;1:138-142.
25. Lind S E, Smith C J. Actin accelerates plasmin generation by tissue plasminogen activator. J Biol Chem. 1991;266:17673-17678.
26. Machovich R, Owen W G. Denatured proteins as cofactors for plasminogen activation. Arch Biochem Biophys. 1997;344:343-349.
27. Machovich R, Ajtai K, Kolev K, Owen W G. Myosin as cofactor and substrate in fibrinolysis. FEBS Lett. 1997;407:93-96.
28. Machovich R, Komorowicz E, Kolev K, Owen W G. Facilitation of plasminogen activation by denatured prothrombin. Thromb Res. 1999;94:389-394.
29. Parkkinen J, Hacker J, Korhonen T K. Enhancement of tissue plasminogen activator-catalyzed plasminogen activation by Escherichia coli S fimbriae associated with neonatal septicaemia and meningitis. Thromb Haemost. 1991 ;65:483-486.
30. Parkkinen J, Rauvala H. Interactions of plasminogen and tissue plasminogen activator (t-PA) with amphoterin. Enhancement of t-PA-catalyzed plasminogen activation by amphoterin. J Biol Chem. 1991;266:16730-16735.
31. Pryzdial E L, Bajzar L, Nesheim M E. Prothrombinase components can accelerate tissue plasminogen activator-catalyzed plasminogen activation. J Biol Chem. 1995;270:17871-17877.
32. Radcliffe R, Heinze T. Stimulation of tissue plasminogen activator by denatured proteins and fibrin clots: a possible additional role for plasminogen activator? Arch Biochem Biophys. 1981;211:750-761.
33. Sheng S, Truong B, Fredrickson D, Wu R, Pardee A B, Sager R. Tissue-type plasminogen activator is a target of the tumor suppressor gene maspin. Proc Natl Acad Sci U S A. 1998;95:499-504.
34. Silverstein R L, Leung L L, Harpel P C, Nachman R L. Complex formation of platelet thrombospondin with plasminogen. Modulation of activation by tissue activator. J Clin Invest. 1984;74:1625-1633.
35. Stack S, Gonzalez-Gronow M, Pizzo S V. Regulation of plasminogen activation by components of the extracellular matrix. Biochemistry. 1990;29:4966-4970.
36. Wada H, Kumeda Y, Ogasawara Z, et al. Stimulation of tissue type plasminogen activator by leukaemic cell homogenates. Blood Coagul Fibrinolysis. 1993;4:591-597.
37. Carrell R W, Gooptu B. Conformational changes and disease—serpins, prions and Alzheimer's. Curr Opin Struct Biol. 1998;8:799-809.
38. Krieger M, Stern DM. Series introduction: multiligand receptors and human disease. J Clin Invest. 2001;108:645-647.
39. Schmidt A M, Yan S D, Wautier J L, Stern D. Activation of receptor for advanced glycation end products: a mechanism for chronic vascular dysfimction in diabetic vasculopathy and atherosclerosis. Circ Res. 1999;84:489-497.
40. Schmidt A M, Yan S D, Yan S F, Stem D M. The biology of the receptor for advanced glycation end products and its ligands. Biochim Biophys Acta. 2000;1498:99-111.
41. Schmidt A M, Hofmann M, Taguchi A, Yan S D, Stem D M. RAGE: a multiligand receptor contributing to the cellular response in diabetic vasculopathy and inflammation. Semin Thromb Hemost. 2000;26:485-493.
42. Schmidt A M, Yan S D, Yan S F, Stern D M. The multiligand receptor RAGE as a progression factor amplifying immune and inflammatory responses. J Clin Invest. 2001 ;108:949-955.
43. Weldon D T, Maggio J E, Mantyh P W. New insights into the neuropathology and cell biology of Alzheimer's disease. Geriatrics. 1997;52 Suppl 2:S13-S16.
44. Yan S D Roher A, Chaney M, Zlokovic B, Schmidt A M, Stem D. Cellular cofactors potentiating induction of stress and cytotoxicity by amyloid beta-peptide. Biochim Biophys Acta. 2000;1502:145-157.
45. Balbach J J, Ishii Y, Antzutkin O N, et al. Amyloid fibril formation by A beta 16-22, a seven-residue fragment of the Alzheimer's beta-amyloid peptide, and structural characterization by solid state NMR. Biochemistry. 2000;39:13748-13759.
46. Bobbink I W, de Boer H C, Tekelenburg W L, Banga J D, de Groot P G. Effect of extracellular matrix glycation on endothelial cell adhesion and spreading: involvement of vitronectin. Diabetes. 1997;46:87-93.
47. Boehm T, Folkman J, Browder T, O'Reilly M S. Antiangiogenic therapy of experimental cancer does not induce acquired drug resistance. Nature. 1997;390:404-407.
48. Andrade M A, Chacon P, Merelo J J, Moran F. Evaluation of secondary structure of proteins from UV circular dichroism spectra using an unsupervised learning neural network. Protein Eng. 1993;6:383-390.
49. Kranenburg O, Bouma B, Kroon-Batenburg L M, et al. Tissue-type plasminogen activator is a multiligand cross-beta structure receptor. Curr Biol. 2002;12:1833-1839.
50. van Zonneveld A J, Veerman H, Pannekoek H. On the interaction of the finger and the kringle-2 domain of tissue-type plasminogen activator with fibrin. Inhibition of kringle-2 binding to fibrin by epsilon-amino caproic acid. J Biol Chem. 1986;261:14214-14218.
51. LeVine H, III. Quantification of beta-sheet amyloid fibril structures with thioflavin T. Methods Enzymol. 1999;309:274-84.:274-284.
52. Kranenburg O, Scharnhorst V, Van der Eb A J, Zantema A. Inhibition of cyclin-dependent kinase activity triggers neuronal differentiation of mouse neuroblastoma cells. J Cell Biol. 1995;131:227-234.
53. Johannessen M, Diness V, Pingel K, et al. Fibrin affinity and clearance of t-PA deletion and substitution analogues. Thromb Haemost. 1990;63:54-59.
54. Gebbink M F, Zondag G C, Koningstein G M, Feiken E, Wubbolts R W, Moolenaar W H. Cell surface expression of receptor protein tyrosine phosphatase RPTP mu is regulated by cell-cell contact. J Cell Biol. 1995;131:251-260.
55. Stewart R J, Fredenburgh J C, Weitz J I. Characterization of the interactions of plasminogen and tissue and vampire bat plasminogen activators with fibrinogen, fibrin, and the complex of D-dimer noncovalently linked to fragment E. J Biol Chem. 1998;273:18292-18299.
56. Tucker H M, Kihiko M, Caldwell J N, et al. The plasmin system is induced by and degrades amyloid-beta aggregates. J Neurosci. 2000;20:3937-3946.
57. Tucker H M, Kihiko-Ehmann M, Wright S, Rydel R E, Estus S. Tissue plasminogen activator requires plasminogen to modulate amyloid-beta neurotoxicity and deposition. J Neurochem. 2000;75 :2172-2177.
58. Redlitz A, Tan A K, Eaton D L, Plow E F. Plasma carboxypeptidases as regulators of the plasminogen system. J Clin Invest. 1995;96:2534-2538.
59. Hayden M R, Tyagi S C. “A” is for amylin and amyloid in type 2 diabetes mellitus. JOP. 2001;2:124-139.
60. Moriarty D F, Raleigh D P. Effects of sequential proline substitutions on amyloid formation by human amylin20-29. Biochemistry. 1999;38:1811-1818.
61. Saishoji T, Higashi T, Ikeda K, et al. Advanced glycation end products stimulate plasminogen activator activity via GM-CSF in RAW 264.7 cells. Biochem Biophys Res Commun. 1995;217:278-285.
62. Nguyen J T, Inouye H, Baldwin M A, et al. X-ray diffraction of scrapie prion rods and PrP peptides. J Mol Biol. 1995;252:412-422.
63. Motomiya Y, Oyama N, Iwamoto H, Uchimura T, Maruyama I. N epsilon-(carboxymethyl)lysine in blood from maintenance hemodialysis patients may contribute to dialysis-related amyloidosis. Kidney Int. 1998;54:1357-1366.
64. Sady C, Jiang C L, Chellan P, et al. Maillard reactions by alpha-oxoaldehydes: detection of glyoxal-modified proteins. Biochim Biophys Acta. 2000;1481:255-264.
65. Shibayama Y, Joseph K, Nakazawa Y, Ghebreihiwet B, Peerschke E I, Kaplan A P. Zinc-dependent activation of the plasma kinin-forming cascade by aggregated beta amyloid protein. Clin Immunol. 1999;90:89-99.
66. Bennett W F, Paoni N F, Keyt B A, et al. High resolution analysis of functional determinants on human tissue-type plasminogen activator. J Biol Chem. 1991;266:5191-5201.
67. Horrevoets A J, Smilde A, de Vries C, Pannekoek H. The specific roles of finger and kringle 2 domains of tissue-type plasminogen activator during in vitro fibrinolysis. J Biol Chem. 1994;269: 12639-12644.
68. Matsuka Y V, Medved L V, Brew S A, Ingham K C. The NH2-terminal fibrin-binding site of fibronectin is formed by interacting fourth and fifth finger domains. Studies with recombinant finger fragments expressed in Escherichia coli. J Biol Chem. 1994;269:9539-9546.
69. Beckmann R, Geiger M, de Vries C, Pannekoek H, Binder B R. Fibronectin decreases the stimulatory effect of fibrin and fibrinogen fragment FCB-2 on plasmin formation by tissue plasminogen activator. J Biol Chem. 1991;266:2227-2232.
70. O'Nuallain B, Wetzel R. Conformational Abs recognizing a generic amyloid fibril epitope. Proc Natl Acad Sci U S A. 2002;99:1485-1490.
71. Kayed R, Head E, Thompson J L, et al. Common structure of soluble amyloid oligomers implies common mechanism of pathogenesis. Science. 2003;300:486-489.
72. Cutler P, Brown F, Camilleri P, et al. The recognition of haemoglobin by antibodies raised for the immunoassay of beta-amyloid. FEBS Lett. 1997;412:341-345.
73. Stem R A, Trojanowski J Q, Lee V M. Antibodies to the beta-amyloid peptide cross-react with conformational epitopes in human fibrinogen subunits from peripheral blood. FEBS Lett. 1990;264:43-47.
74. Downing A K, Driscoll P C, Harvey T S, et al. Solution structure of the fibrin binding finger domain of tissue-type plasminogen activator determined by 1H nuclear magnetic resonance. J Mol Biol. 1992;225:821-833.
75. Smith B O, Downing A K, Driscoll P C, Dudgeon T J, Campbell I D. The solution structure and backbone dynamics of the fibronectin type I and epidermal growth factor-like pair of modules of tissue-type plasminogen activator. Structure. 1995;3:823-833.
76. Williams M J, Phan I, Harvey T S, Rostagno A, Gold L I, Campbell I D. Solution structure of a pair of fibronectin type 1 modules with fibrin binding activity. J Mol Biol. 1994;235:1302-1311.
Bamberger, M. E., Harris, M. E., McDonald, D. R., Husemann, J., Landreth, G. E., 2003. A cell surface receptor complex for fibrillar beta-amyloid mediates microglial activation. J. Neurosci., 23, 2665-2674.
Beckmann, R., Geiger, M., de Vries, C., Pannekoek, H., Binder, B. R., 1991. Fibronectin decreases the stimulatory effect of fibrin and fibrinogen fragment FCB-2 on plasmin formation by tissue plasminogen activator. J. Biol. Chem., 266, 2227-2232.
Bellotti, V., Mangione, P., Merlini, G., 2000. Review: immunoglobulin light chain amyloidosis—the archetype of structural and pathogenic variability. J. Struct. Biol., 130, 280-289.
Bouma, B., Kroon-Batenburg, L. M., Wu, Y. P., Brunjes, B., Posthuma, G., Kranenburg, O., de Groot, P. G., Voest, E. E., Gebbink, M. F., 2003. Glycation induces formation of amyloid cross-beta structure in albumin. J. Biol. Chem., 278, 41810-41819.
Buxbaum, J. N., 2004. The systemic amyloidoses. Curr. Opin. Rheumatol., 16, 67-75.
Claessen, D., Rink, R., de Jong, W., Siebring, J., de Vreugd, P., Boersma, F. G., Dijkhuizen, L., Wosten, H. A., 2003a. A novel class of secreted hydrophobic proteins is involved in aerial hyphae formation in Streptomyces coelicolor by forming amyloid-like fibrils. Genes Dev., 17, 1714-1726.
Claessen, D., Rink, R., de Jong, W., Siebring, J., de Vreugd, P., Boersma, F. G., Dijkhuizen, L., Wosten, H. A., 2003b. A novel class of secreted hydrophobic proteins is involved in aerial hyphae formation in Streptomyces coelicolor by forming amyloid-like fibrils. Genes Dev., 17, 1714-1726.
Connor, P., Hunt, B. J., 2003. Cerebral haemostasis and antiphospholipid antibodies. Lupus, 12, 929-934.
Coraci, I. S., Husemann, J., Berman, J. W., Hulette, C., Dufour, J. H., Campanella, G. K., Luster, A. D., Silverstein, S. C., El Khoury, J. B., 2002. CD36, a class B scavenger receptor, is expressed on microglia in Alzheimer's disease brains and can mediate production of reactive oxygen species in response to beta-amyloid fibrils. Am. J. Pathol., 160, 101-112.
de Groot, P. G., Derksen, R. H., 2004. Antiphospholipid antibodies: update on detection, pathophysiology, and treatment. Curr. Opin. Hematol., 11, 165-169.
de Groot, P. G., Horbach, D. A., Derksen, R. H., 1996. Protein C and other cofactors involved in the binding of antiphospholipid antibodies: relation to the pathogenesis of thrombosis. Lupus, 5, 488-493.
de Laat, B., Derksen, R. H., Urbanus, R. T., de Groot, P. G., 2004a. IgG antibodies that recognize epitope Gly40-Arg43 in domain I of {beta}2-glycoprotein I cause LAC and their presence correlates strongly with thrombosis. Blood,.
de Laat, H.B., Derksen, R. H., Urbanus, R. T., Roest, M., de Groot, P. G., 2004b. beta2-glycoprotein I-dependent lupus anticoagulant highly correlates with thrombosis in the antiphospholipid syndrome. Blood, 104, 3598-3602.
Gamba, G., Montani, N., Anesi, E., Palladini, G., Capezzera, M., Soldavini, E., Merlini, G., 2000. Clotting alterations in primary systemic amyloidosis. Haematologica, 85, 289-292.
Gebbink, M. F., Zondag, G. C., Koningstein, G. M., Feiken, E., Wubbolts, R. W., Moolenaar, W. H., 1995. Cell surface expression of receptor protein tyrosine phosphatase RPTP mu is regulated by cell-cell contact. J. Cell Biol., 131, 251-260.
Hackeng, T. M., Dawson, P. E., Kent, S. B., Griffin, J. H., 1998. Chemical synthesis of human protein S thrombin-sensitive module and first epidermal growth factor module. Biopolymers, 46, 53-63.
Hackeng, T. M., Fernandez, J. A., Dawson, P. E., Kent, S. B., Griffin, J. H., 2000. Chemical synthesis and spontaneous folding of a multidomain protein: anticoagulant microprotein S. Proc. Natl. Acad. Sci. U. S. A, %19;97, 14074-14078.
Hackeng, T. M., Griffin, J. H., Dawson, P. E., 1999. Protein synthesis by native chemical ligation: expanded scope by using straightforward methodology. Proc. Natl. Acad. Sci. U. S. A, 96, 10068-10073.
Hackeng, T. M., Mounier, C. M., Bon, C., Dawson, P. E., Griffin, J. H., Kent, S. B., 1997. Total chemical synthesis of enzymatically active human type II secretory phospholipase A2. Proc. Natl. Acad. Sci. U. S. A, 94, 7845-7850.
Hackeng, T. M., Rosing, J., Spronk, H. M., Vermeer, C., 2001. Total chemical synthesis of human matrix Gla protein. Protein Sci., 10, 864-870.
Hoppener, J. W., Ahren, B., Lips, C. J., 2000. Islet amyloid and type 2 diabetes mellitus. N. Engl. J. Med., 343, 411-419.
Hoppener, J. W., Oosterwijk, C., Nieuwenhuis, M. G., Posthuma, G., Thijssen, J. H., Vroom, T. M., Ahren, B., Lips, C. J., 1999. Extensive islet amyloid formation is induced by development of Type II diabetes mellitus and contributes to its progression: pathogenesis of diabetes in a mouse model. Diabetologia, 42, 427-434.
Horbach, D. A., van Oort, E., Donders, R .C., Derksen, R. H., de Groot, P. G., 1996. Lupus anticoagulant is the strongest risk factor for both venous and arterial thrombosis in patients with systemic lupus erythematosus. Comparison between different assays for the detection of antiphospholipid antibodies. Thromb. Haemost., 76, 916-924.
Horbach, D. A., van Oort, E., Tempelman, M. J., Derksen, R. H., de Groot, P. G., 1998. The prevalence of a non-phospholipid-binding form of beta2-glycoprotein I in human plasma—consequences for the development of anti-beta2-glycoprotein I antibodies. Thromb. Haemost., 80, 791-797.
Isobe, T., 1993. AA amyloidosis and AL amyloidosis. Intern. Med., 32, 919-920.
Jaroniec, C. P., MacPhee, C. E., Astrof, N. S., Dobson, C. M., Griffin, R. G., 2002. Molecular conformation of a peptide fragment of transthyretin in an amyloid fibril. Proc. Natl. Acad. Sci. U. S. A, 99, 16748-16753.
Johannessen, M., Diness, V., Pingel, K., Petersen, L. C., Rao, D., Lioubin, P., O'Hara, P., Mulvihill, E., 1990. Fibrin affinity and clearance of t-PA deletion and substitution analogues. Thromb. Haemost., %19;63, 54-59.
Kranenburg, O., Bouma, B., Kroon-Batenburg, L. M., Reijerkerk, A., Wu, Y. P., Voest, E. E., Gebbink, M. F., 2002. Tissue-type plasminogen activator is a multiligand cross-beta structure receptor. Curr. Biol., 12, 1833-1839.
Kranenburg, O., Kroon-Batenburg, L. M., Reijerkerk, A., Wu, Y. P., Voest, E. E., Gebbink, M. F., 2003. Recombinant endostatin forms amyloid fibrils that bind and are cytotoxic to murine neuroblastoma cells in vitro. FEBS Lett., 539, 149-155.
Kurusu, A., Yamada, T., Yamaji, K., Nishitani, M., Tashiro, K., Maeda, K., Horikoshi, S., Shirato, I., Rinno, H., Tomino, Y., 2004. A case of primary immunoglobulin light chain amyloidosis with a delayed appearance of Bence Jones protein in urine. Nephrology. (Carlton. ), 9, 122-125.
Matsuura, E., Igarashi, Y., Yasuda, T., Triplett, D. A., Koike, T., 1994. Anticardiolipin antibodies recognize beta 2-glycoprotein I structure altered by interacting with an oxygen modified solid phase surface. J. Exp. Med., 179, 457-462.
Matzinger, P., 2002a. An innate sense of danger. Ann. N. Y. Acad. Sci., 961:341-2., 341-342.
Matzinger, P., 2002b. The danger model: a renewed sense of self. Science, 296, 301-305.
Ohgami, N., Nagai, R., Ikemoto, M., Arai, H., Kuniyasu, A., Horiuchi, S., Nakayama, H., 2001. CD36, a member of class B scavenger receptor family, is a receptor for advanced glycation end products. Ann. N. Y. Acad. Sci., 947:350-5., 350-355.
Pepys, M. B., 2001. Pathogenesis, diagnosis and treatment of systemic amyloidosis. Philos. Trans. R. Soc. Lond B Biol. Sci., 356, 203-210.
Redegeld, F. A., Nijkamp, F. P., 2003. Immunoglobulin free light chains and mast cells: pivotal role in T-cell-mediated immune reactions? Trends Immunol., 24, 181-185.
Redegeld, F. A., van der Heijden, M. W., Kool, M., Heijdra, B. M., Garssen, J., Kraneveld, A. D., Van Loveren, H., Roholl, P., Saito, T., Verbeek, J. S., Claassens, J., Koster, A. S., Nijkamp, F. P., 2002. Immunoglobulin-free light chains elicit immediate hypersensitivity-like responses. Nat. Med., 8, 694-701.
Reijerkerk, A., Mosnier, L. O., Kranenburg, O., Bouma, B. N., Carmeliet, P., Drixler, T., Meijers, J. C., Voest, E. E., Gebbink, M. F., 2003. Amyloid endostatin induces endothelial cell detachment by stimulation of the plasminogen activation system. Mol. Cancer Res., 1, 561-568.
Rostagno, A. A., Schwarzbauer, J. E., Gold, L. I., 1999. Comparison of the fibrin-binding activities in the N- and C-termini of fibronectin. Biochem. J., 338, 375-386.
Schielen, J. G., Adams, H. P., Voskuilen, M., Tesser, G. J., Nieuwenhuizen, W., 1991. Structural requirements of position A alpha-157 in fibrinogen for the fibrin-induced rate enhancement of the activation of plasminogen by tissue-type plasminogen activator. Biochem. J., 276, 655-659.
Solomon, A., 1986. Light chains of immunoglobulins: structural-genetic correlates. Blood, 68, 603-610.
Subang, R., Levine, J. S., Janoff, A. S., Davidson, S. M., Taraschi, T. F., Koike,T., Minchey, S. R., Whiteside, M., Tannenbaum, M., Rauch, J., 2000. Phospholipid-bound beta 2-glycoprotein I induces the production of anti-phospholipid antibodies. J. Autoimmun., 15, 21-32.
Ursini, F., Davies, K. J., Maiorino, M., Parasassi, T., Sevanian, A., 2002. Atherosclerosis: another protein misfolding disease? Trends Mol. Med., 8, 370-374.
van Zonneveld, A. J., Veerman, H., Pannekoek, H., 1986. On the interaction of the finger and the kringle-2 domain of tissue-type plasminogen activator with fibrin. Inhibition of kringle-2 binding to fibrin by epsilon-amino caproic acid. J. Biol. Chem., 261, 14214-14218.
Yamada, M., Kadoya, Y., Kasai, S., Kato, K., Mochizuki, M., Nishi, N., Watanabe, N., Kleinman, H. K., Yamada, Y., Nomizu, M., 2002. Ile-Lys-Val-Ala-Val (IKVAV)-containing laminin alphal chain peptides form amyloid-like fibrils. FEBS Lett., 530, 48-52.

1. A method for modulating extracellular degradation and/or clearance of a protein in a subject's circulation, said method comprising:
modulating cross-β structure formation of protein present in the circulation.

2-22. (canceled)

23. A method for modulating extracellular degradation and/or clearance of a protein, said method comprising:
modulating an interaction of a compound comprising tissue plasminogen activator (tPA)-like activity and the substrate of said activity.

24. A method for modulating extracellular degradation and/or clearance of a protein, said method comprising:
modulating the activity of a receptor for cross-β forming proteins.

25-52. (canceled)

53. A method for interfering in coagulation of blood, said method comprising:
providing to the blood a binding molecule that either binds to a cross-β structure or to a compound comprising a specific binding partner of cross-β wherein said compound is part of a blood coagulation cascade.

54. (canceled)

55. The method according to claim 53, wherein said compound which is part of a blood-coagulating cascade is selected from the group consisting of a platelet, fibrin, Factor XII, and combinations thereof.

56. (canceled)

57. The method according to claim 53, wherein said cross-β specific binding partner is selected from the group consisting of CD36, LRP, apoER2′, scavenger receptor A, scavenger receptor B-I, and combinations thereof.

58. The method according to claim 53, wherein said specific binding partner is selected from the group consisting of CD36, LRP, scavenger receptor A, scavenger receptor B-I, RAGE, FEEL-1, FEEL-2, SREC-1, LOX-1, stabilin-1, stabilin-2, and combinations thereof.

59. The method according to claim 53, wherein said bi-specific molecule is an antibody.

60-63. (canceled)


SEQUENCE LISTING (45 sequences)

SEQ ID NO: 1 - protein, 28 aa, Artificial sequence
  Part of human tpA
  misc_feature (2)..(2): Xaa can be any naturally occurring amino acid
  misc_feature (4)..(16): Xaa can be any naturally occurring amino acid
  misc_feature (18)..(25): Xaa can be any naturally occurring amino acid
  misc_feature (27)..(27): Xaa can be any naturally occurring amino acid
RXEXXXXXXXXXXXXXRXXXXXXXXEXR

SEQ ID NO: 2 - protein, 8 aa, Artificial sequence
  Part of human fibronectin
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
RXEXKXER

SEQ ID NO: 3 - protein, 50 aa, Artificial sequence
  Finger domain of human tpA
SYQVICRDEKTQMIYQQHQSWLRPVLRSNRVEYCWCNSGRAQCHSVPVKS

SEQ ID NO: 4 - protein, 47 aa, Artificial sequence
  Finger domain of human FXII
QKEKCFEPQLLRFFHKNDIWYRTEQAAVARCQCKGPEAHCQRLASQA

SEQ ID NO: 5 - protein, 47 aa, Artificial sequence
  Finger domain of human HGFa
GTEKCFDETRYEYLEGGDRWARVRQGHVEQCECFGGRTWCEGTRHWA

SEQ ID NO: 6 - protein, 44 aa, Artificial sequence
  Finger domain of human FN1-1
SKPGCYDNGKHYQINQQWERTYLGNVLVCTCYGGSRGFNCESKP

SEQ ID NO: 7 - protein, 49 aa, Artificial sequence
  Finger domain of human FN1-2
EAEETCFDKYTGNTYRVGDTYERPKDSMIWDCTCIGAGRGRISCTIANR

SEQ ID NO: 8 - protein, 50 aa, Artificial sequence
  Finger domain of human FN1-3
TIANRCHEGGQSYKIGDTWRRPHETGGYMLECVCLGNGKGEWTCKPIAEK

SEQ ID NO: 9 - protein, 51 aa, Artificial sequence
  Finger domain of human FN1-4
KPIAEKCFDHAAGTSYVVGETWEKPYNGWMMVDCTCLGEGSGRITCTSRNR

SEQ ID NO: 10 - protein, 52 aa, Artificial sequence
  Finger domain of human FN1-5
TSRNRCNDQDTRTSYRIGDTWSKKDNRGNLLQCICTGNGRGEWKCERHTSVQ

SEQ ID NO: 11 - protein, 45 aa, Artificial sequence
  Finger domain of human FN1-6
PPYGHCVTDSGVVYSVGMQWLKTQGNKQMLCTCLGNGVSCQETAV

SEQ ID NO: 12 - protein, 56 aa, Artificial sequence
  Finger domain of human FN1-7
PMAAHEEICTTNEGVMYRIGDNWDKQHDMGHMMRRCTCVGNDRGEWTCIAYSQLRD

SEQ ID NO: 13 - protein, 52 aa, Artificial sequence
  Finger domain of human FN1-8
IAYSNLRDQCIVDDITYNVNDTFHKRHEEGHMLNCTCFGQGRGRWKCDPVDQ

SEQ ID NO: 14 - protein, 54 aa, Artificial sequence
  Finger domain of human FN1-9
WKCDPVDQCQDSETGTFQIGDSWEKYVHGVRYQCYCYGRGIGEWHCQPLQTYPS

SEQ ID NO: 15 - protein, 53 aa, Artificial sequence
  Finger domain of human FN1-10
NQPTDDSCFDPYTVSHYAVGDEWERMSSESGFKLLCQCLGFGSGHFRCDSSRW

SEQ ID NO: 16 - protein, 49 aa, Artificial sequence
  Finger domain of human FN1-11
DSSRWCHDNGVNYKIGEKWDRQGENGQMMSCTCLGNGKGEFKCDPHEAT

SEQ ID NO: 17 - protein, 50 aa, Artificial sequence
  Finger domain of human FN1-12
DPHEATCYDDGKTYGVGEQWQKEYLGAICSCTCFGGQRGWRCDNCRRPGG

SEQ ID NO: 18 - protein, 40 aa, Artificial sequence
  Peptide A Beta (1-40) from Homo sapiens
DAEFRHDSGYEVHHQKLVFFAEDVGSNKGAIIGLMVGGVV

SEQ ID NO: 19 - protein, 13 aa, Artificial sequence
  Peptide FP13 from Homo sapiens
KRLEVDIDIKIRS

SEQ ID NO: 20 - protein, 12 aa, Artificial sequence
  Peptide FP12 from Homo sapiens
KRLEVDIDIKIR

SEQ ID NO: 21 - protein, 10 aa, Artificial sequence
  Peptide FP10 from Homo sapiens
KRLEVDIDIK

SEQ ID NO: 22 - protein, 37 aa, Artificial sequence [sequence omitted]

SEQ ID NO: 23 - protein, 10 aa, Artificial sequence
  Peptide Delta hIAPP from Homo sapiens
SNNFGAILSS

SEQ ID NO: 24 - protein, 10 aa, Artificial sequence
  Peptide Delta mIAPP from Homo sapiens
SNNLGPVLPP

SEQ ID NO: 25 - DNA, 38 bp, Artificial sequence
  Primer
aaaagtcgacagccgccaccatggatgcaatgaagaga

SEQ ID NO: 26 - DNA, 33 bp, Artificial sequence
  Primer
aaaagcggccgcccacttttgacaggcactgag

SEQ ID NO: 27 - DNA, 32 bp, Artificial sequence
  Primer
aaaagcggccgcgtggccctggtatctatttc

SEQ ID NO: 28 - DNA, 29 bp, Artificial sequence
  Primer
aaaagagatctgtgcctgtcaaaagttgc

SEQ ID NO: 29 - DNA, 32 bp, Artificial sequence
  Primer
aaaagagatctgataccagggccacgtgctac

SEQ ID NO: 30 - DNA, 35 bp, Artificial sequence
  Primer
aaaagcggccgcccgtcactgtttccctcagagca

SEQ ID NO: 31 - DNA, 30 bp, Artificial sequence
  Primer
aaagcggccgcctggctcctcttctgaatc

SEQ ID NO: 32 - DNA, 31 bp, Artificial sequence
  Primer
tgcaagatctatagctgagaagtgttttgat

SEQ ID NO: 33 - DNA, 35 bp, Artificial sequence
  Primer
gatgcggccgccctgtattcctagaagtgcaagtg

SEQ ID NO: 34 - DNA, 31 bp, Artificial sequence
  Primer
tgcaagatctacttctagaaatagatgcaac

SEQ ID NO: 35 - DNA, 32 bp, Artificial sequence
  Primer
tgatgcggccgccccacagaggtgtgcctctc

SEQ ID NO: 36 - DNA, 31 bp, Artificial sequence
  Primer
aaaaaagatctaaccaacctacggatgactc

SEQ ID NO: 37 - DNA, 32 bp, Artificial sequence
  Primer
aaaaaaggtaccgactgggttcacccccaggt

SEQ ID NO: 38 - DNA, 32 bp, Artificial sequence
  Primer
gaaacaagatctcagaaagagaagtgctttga

SEQ ID NO: 39 - DNA, 34 bp, Artificial sequence
  Primer
acgggcggccgcccggcctggctggccagccgct

SEQ ID NO: 40 - DNA, 31 bp, Artificial sequence
  Primer
aaaaaagatctcagaaagagaagtgctttga

SEQ ID NO: 41 - DNA, 31 bp, Artificial sequence
  Primer
aaaaaggtaccggcttgccttggtgtccacg

SEQ ID NO: 42 - DNA, 32 bp, Artificial sequence
  Primer
gcaagaagatctggcacagagaaatgctttga

SEQ ID NO: 43 - DNA, 34 bp, Artificial sequence
  Primer
aagggcggccgcccagctgtatgtcgggtgcctt

SEQ ID NO: 44 - DNA, 30 bp, Artificial sequence
  Primer
aaaaaagatctggcacagagaatgctttga

SEQ ID NO: 45 - DNA, 31 bp, Artificial sequence
  Primer
aaaaaggtaccgctcatcaggctcgatgttg